SEQUENCE LISTING (21 sequences)

SEQ ID NO: 1 - DNA, 20 bp, synthetic construct
  source 1..20: mol_type = other DNA
aatgatacggcgaccaccga

SEQ ID NO: 2 - DNA, 29 bp, synthetic construct
  source 1..29: mol_type = other DNA
aatgatacggcgaccaccgagatctacac

SEQ ID NO: 3 - DNA, 20 bp, synthetic construct
  source 1..20: mol_type = other DNA
caagcagaagacggcatacg

SEQ ID NO: 4 - DNA, 24 bp, synthetic construct
  source 1..24: mol_type = other DNA
caagcagaagacggcatacgagat

SEQ ID NO: 5 - DNA, 20 bp, synthetic construct
  source 1..20: mol_type = other DNA
tcggtggtcgccgtatcatt

SEQ ID NO: 6 - DNA, 29 bp, synthetic construct
  source 1..29: mol_type = other DNA
gtgtagatctcggtggtcgccgtatcatt

SEQ ID NO: 7 - DNA, 20 bp, synthetic construct
  source 1..20: mol_type = other DNA
cgtatgccgtcttctgcttg

SEQ ID NO: 8 - DNA, 24 bp, synthetic construct
  source 1..24: mol_type = other DNA
atctcgtatgccgtcttctgcttg

SEQ ID NO: 9 - DNA, 15 bp, synthetic construct
  source 1..15: mol_type = other DNA
gtctcgtgggctcgg

SEQ ID NO: 10 - DNA, 15 bp, synthetic construct
  source 1..15: mol_type = other DNA
ccgagcccacgagac

SEQ ID NO: 11 - DNA, 35 bp, synthetic construct
  source 1..35: mol_type = other DNA
ttttttaatgatacggcgaccaccgaganctacac

SEQ ID NO: 12 - DNA, 33 bp, synthetic construct
  source 1..33: mol_type = other DNA
ttttttnnncaagcagaagacggcatacgagat

SEQ ID NO: 13 - DNA, 33 bp, synthetic construct
  source 1..33: mol_type = other DNA
acactctttccctacacgacgctcttccgatct

SEQ ID NO: 14 - DNA, 20 bp, synthetic construct
  source 1..20: mol_type = other DNA
acactctttccctacacgac

SEQ ID NO: 15 - DNA, 34 bp, synthetic construct
  source 1..34: mol_type = other DNA
gtgactggagttcagacgtgtgctcttccgatct

SEQ ID NO: 16 - DNA, 21 bp, synthetic construct
  source 1..21: mol_type = other DNA
gtgactggagttcagacgtgt

SEQ ID NO: 17 - DNA, 30 bp, synthetic construct
  source 1..30: mol_type = other DNA
  modified_base 1: mod_base = OTHER; note = Phosphorothioate
ttttttttttaatgatacggcgaccaccga

SEQ ID NO: 18 - DNA, 31 bp, synthetic construct
  source 1..31: mol_type = other DNA
  modified_base 1: mod_base = OTHER; note = Phosphorothioate
ttttttttttcaagcagaagacggcatacga

SEQ ID NO: 19 - DNA, 34 bp, synthetic construct; identical to SEQ ID NO: 15
  source 1..34: mol_type = other DNA

SEQ ID NO: 20 - DNA, 33 bp, synthetic construct
  source 1..33: mol_type = other DNA
agatcggaagagcgtcgtgtagggaaagagtgt

SEQ ID NO: 21 - DNA, 24 bp, synthetic construct
  source 1..24: mol_type = other DNA
aggaggaggaggaggaggaggagg

(19) United States
(12) Patent Application Publication
(10) Pub. No.: US 2025/0369156 A1
(43) Pub. Date: Dec. 4, 2025

(54) SPATIAL TRANSCRIPTOMICS LIBRARY PREPARATION MATERIALS AND METHODS

(71) Applicant: ILLUMINA, INC., San Diego, CA (US)

(72) Inventors: Andrew Ostrow, San Diego, CA (US); Samuel Clamons, San Diego, CA (US); Junlian Hu, Vista, CA (US); Mats Ekstrand, San Diego, CA (US); Jonathan Manthe, San Diego, CA (US); Craig April, San Diego, CA (US); Andrea Manzo, San Diego, CA (US); Jeffrey Fisher, San Diego, CA (US); Brian Mather, San Diego, CA (US); Fiona Kaper, Solana Beach, CA (US); Adam White, San Diego, CA (US)

(21) Appl. No.: 18/875,209

(22) PCT Filed: Dec. 29, 2023
(86) PCT No.: PCT/US2023/086422

§ 371 Date: Dec. 16, 2024

Related U.S. Application Data
(60) Provisional application No. 63/477,726, filed on Dec. 29, 2022.
(60) Provisional application No. 63/612,819, filed on Dec. 20, 2023.

(51) Int. Cl. C40B 50/00; C12Q 1/48; C12Q 1/6806; C12Q 1/6813; C12Q 1/6855; C12Q 1/686; C12Q 1/6869; C12Q 1/6886; C40B 20/00

(52) U.S. Cl. CPC C40B 50/00; C12Q 1/48; C12Q 1/6806; C12Q 1/6813; C12Q 1/6855; C12Q 1/686; C12Q 1/6869; C12Q 1/6886; C12Y 207/07; C40B 20/00

(57) ABSTRACT

The present disclosure relates, in general, to methods for improving preparation of a spatial transcriptomics RNA, library, for example a mRNA library, by improving capture of RNA transcript information from a tissue sample in situ. The spatial transcriptomics library from a tissue sample is useful to determine a genetic profile and help diagnose a person who has or is at risk of having a disease, such as cancer, genetic disease, autoimmune disease, and other indications, and improve treatment of the subject.

### CROSS REFERENCE TO RELATED APPLICATIONS

The present application claims the priority benefit of U.S. Provisional Patent Application No. 63/477,726, filed Dec. 29, 2022, and U.S. Provisional Patent Application No. 63/612,819, filed Dec. 20, 2023, incorporated by reference herein in their entireties.

### INCORPORATION BY REFERENCE OF SEQUENCE DISCLOSURE

The Sequence Listing, which is a part of the present disclosure, is submitted concurrently with the specification as a computer readable file. The name of the file containing the Sequence Listing is “IP-2535-PC_SeqListing.xml”, which was created on Dec. 21, 2023, and is 20,735 bytes in size. The subject matter of the Sequence Listing is incorporated herein in its entirety by reference.

### FIELD OF THE DISCLOSURE

The present disclosure, relates, in general, to methods for generating a spatial transcriptomics mRNA library by improving methods of capturing mRNA transcripts from in situ samples, and mRNA libraries made by these methods.

### BACKGROUND

Spatial transcriptomics enables high resolution in situ gene expression profiling in which cellular relationships are captured within complex tissue architectures. Formalin-fixed, paraffin-embedded (FFPE) tissues represent an invaluable resource for cancer research, as they are the most widely available material for which patient outcomes are known (recent estimates suggest >1 billion FFPE samples worldwide). However, formalin fixation and subsequent de-crosslinking are known to cause degradation and chemical modification of RNAs during tissue processing making poly-A capture of mRNA more challenging than in fresh frozen tissue.

### SUMMARY

The present disclosure provides improved methods for generating a mRNA transcript library from an in situ sample, e.g., fresh frozen or formalin-fixed paraffin embedded tissue sample, by improving efficiency of capture of mRNA transcripts from the tissue sample, thereby generating a more complete transcriptomics library. The method is useful in isolating genomic information from a sample, such as tumor biopsy or other tissue in patients suffering from a disease, and associating the genetic information with having or being at risk of having or developing a disease.

In one aspect, the disclosure provides a method of preparing a mRNA transcript expression library from a tissue sample comprising a) mounting the tissue sample on a substrate comprising a plurality of capture oligonucleotides, wherein the capture oligonucleotides comprise a first clustering sequence (e.g., P7), a spatial barcode sequence (SBC) and a first universal adapter sequence (e.g., Rd2 adapter); b) contacting the tissue sample with i) a plurality of 5′ gene specific probes comprising a sequence complementary to the first universal adapter sequence and a 5′ gene specific primer; and ii) a plurality of 3′ gene specific probes comprising a 3′ gene specific primer, a unique molecular index, and a second universal adapter sequence (e.g., a Rd1 adapter), under conditions such that one or more 5′ gene specific probe and one or more 3′ gene specific probe hybridizes to one or more mRNA transcript in the tissue sample; c) contacting the tissue sample in (b) with ligation reagents such that a 5′ gene specific probe and a 3′ gene specific probe hybridized to the mRNA transcript in proximity to each other are ligated together to form one or more ligated gene specific probe pairs; d) removing the mRNA transcript hybridized to the ligated gene specific probe pairs and leaving a ligated gene specific probe pair oligonucleotide sequence; e) capturing the ligated gene specific probe pair oligonucleotide of (d) on the substrate by binding of the sequence complementary to the first universal adapter sequence in the 5′ gene specific probe to the first universal adapter sequence of the capture oligonucleotide (e.g., Rd2 adapter).

Also contemplated is a method of determining mRNA transcript expression in a tissue sample comprising a) mounting the tissue sample on a substrate comprising a plurality of capture oligonucleotides, wherein the capture oligonucleotides comprise a first clustering sequence (e.g., P7), a spatial barcode sequence (SBC) and a first universal adapter sequence (e.g., Rd2 adapter); b) contacting the tissue sample with i) a plurality of 5 gene specific probes comprising a sequence complementary to the first universal adapter sequence and a 5′ gene specific primer; and ii) a plurality of 3′ gene specific probes comprising a 3′ gene specific primer, a unique molecular index, and a second universal adapter sequence (e.g., Rd1 adapter), under conditions such that one or more 5′ gene specific probe and one or more 3′ gene specific probe hybridizes to one or more mRNA transcript in the tissue sample; c) contacting the tissue sample in (b) with ligation reagents such that a 5′ gene specific probe and a 3′ gene specific probe hybridized to the mRNA transcript in proximity to each other are ligated together to form one or more ligated gene specific probe pairs; d) removing mRNA transcripts hybridized to ligated gene specific probe pairs and leaving ligated gene specific probe pair oligonucleotide sequences; e) capturing the ligated gene specific probe pair oligonucleotide of (d) on the substrate by binding of the sequence complementary to the first universal adapter sequence in the 5′ gene specific probe to the first universal adapter sequence of the capture oligonucleotide (e.g., Rd2 adapter).

In another aspect, the disclosure provides a method of preparing a mRNA transcript expression library from a tissue sample and/or a method of determining mRNA transcript expression from a tissue sample comprising a) mounting the tissue sample on a substrate comprising a plurality of capture oligonucleotides, wherein the capture oligonucleotides comprise a first clustering sequence (e.g., P7), a spatial barcode sequence (SBC) and a first universal adapter sequence (e.g., Rd2 adapter); b) contacting the tissue sample with i) a plurality of 5′ gene specific probes comprising a sequence complementary to the first universal adapter sequence, a unique molecular index, and a 5′ gene specific primer; and ii) a plurality of 3′ gene specific probes comprising a 3′ gene specific primer, and a second universal adapter sequence (e.g., a Rd1 adapter), under conditions such that one or more 5′ gene specific probe and one or more 3′ gene specific probe hybridizes to one or more mRNA transcript in the tissue sample; c) contacting the tissue sample in (b) with ligation reagents such that a 5′ gene specific probe and a 3′ gene specific probe hybridized to the mRNA transcript in proximity to each other are ligated together to form one or more ligated gene specific probe pairs; d) removing the mRNA transcript hybridized to the ligated gene specific probe pairs and leaving a ligated gene specific probe pair oligonucleotide sequence; e) capturing the ligated gene specific probe pair oligonucleotide of (d) on the substrate by binding of the sequence complementary to the first universal adapter sequence in the 5′ gene specific probe to the first universal adapter sequence of the capture oligonucleotide (e.g., Rd2 adapter).

In various embodiments, the disclosure provides a method of preparing a mRNA transcript expression library from a tissue sample comprising a) mounting the tissue sample on a substrate comprising a plurality of capture oligonucleotides, wherein the capture oligonucleotides comprise a first clustering sequence (e.g., P7), a spatial barcode sequence (SBC) and a first universal adapter sequence (e.g., Rd2 adapter); b) contacting the tissue sample with i) a plurality of 5′ gene specific probes comprising a sequence complementary to the first universal adapter sequence and a 5′ gene specific primer; and ii) a plurality of 3′ gene specific probes comprising a 3′ gene specific primer, a unique molecular index, and a second universal adapter sequence (e.g., Rd1 adapter), under conditions such that one or more 5′ gene specific probe and one or more 3′ gene specific probe hybridizes to one or more mRNA transcript in the tissue sample, wherein hybridization of the 5′ gene specific probe and 3′ gene specific probe on the mRNA transcript results in a nucleotide gap between the hybridized molecules; c) contacting the tissue sample in (b) with nucleotide bases and ligation reagents such that the gap between the 5′ gene specific probe and 3° gene specific probe hybridized to the mRNA transcript is filled with nucleotide bases complementary to the mRNA transcript, and a 5′ gene specific probe and a 3′ gene specific probe are ligated together to form one or more ligated gene specific probe pairs; d) removing mRNA transcripts hybridized to ligated gene specific probe pairs and leaving ligated gene specific probe pair oligonucleotide sequences; and e) capturing the ligated gene specific probe pair oligonucleotide sequences of (d) on the substrate by binding of the sequence complementary to the first universal adapter sequence in the 5′ gene specific probe to the first universal adapter sequence of the capture oligonucleotide (e.g., Rd2 adapter).

The disclosure further contemplates a method of determining mRNA transcript expression in a tissue sample comprising a) mounting the tissue sample on a substrate comprising a plurality of capture oligonucleotides, wherein the capture oligonucleotides comprise a first clustering sequence (e.g., P7), a spatial barcode sequence (SBC) and a first universal adapter sequence (e.g., Rd2 adapter); b) contacting the tissue sample with i) a plurality of 5′ gene specific probes comprising a sequence complementary to the first universal adapter sequence and a 5′ gene specific primer; and ii) a plurality of 3′ gene specific probes comprising a 3′ gene specific primer, a unique molecular index, and a second universal adapter sequence (e.g., Rd1 adapter), under conditions such that one or more 5′ gene specific probe and one or more 3′ gene specific probe hybridizes to one or more mRNA transcript in the tissue sample, wherein hybridization of the one or more 5′ gene specific probe and one or more 3′ gene specific probe on the mRNA transcript results in a nucleotide gap between the hybridized molecules; c) contacting the tissue sample in (b) with nucleotide bases and ligation reagents such that the nucleotide gap between a 5′ gene specific probe and a 3′ gene specific probe hybridized to the mRNA transcript is filled with nucleotide bases complementary to the mRNA transcript, and the 5′ gene specific probe and 3′ gene specific probe are ligated together to form one or more ligated gene specific probe pairs; d) removing mRNA transcripts hybridized to the ligated gene specific probe pairs and leaving ligated gene specific probe pair oligonucleotide sequences; and e) capturing the ligated gene specific probe pair oligonucleotide sequences of (d) on the substrate by binding of the sequence complementary to the first universal adapter sequence in the 5′ gene specific probe to the first universal adapter sequence of the capture oligonucleotide (e.g., Rd2 adapter).

In various embodiments, the disclosure provides a method of preparing a mRNA transcript expression library from a tissue sample and/or a method of determining mRNA transcript expression from a tissue sample comprising a) mounting the tissue sample on a substrate comprising a plurality of capture oligonucleotides, wherein the capture oligonucleotides comprise a first clustering sequence (e.g., P7), a spatial barcode sequence (SBC) and a first universal adapter sequence (e.g., Rd2 adapter); b) contacting the tissue sample with i) a plurality of 5′ gene specific probes comprising a sequence complementary to the first universal adapter sequence, a unique molecular index, and a 5′ gene specific primer; and ii) a plurality of 3′ gene specific probes comprising a 3′ gene specific primer and a second universal adapter sequence (e.g., Rd1 adapter), under conditions such that one or more 5′ gene specific probe and one or more 3′ gene specific probe hybridizes to one or more mRNA transcript in the tissue sample, wherein hybridization of the 5′ gene specific probe and 3′ gene specific probe on the mRNA transcript results in a nucleotide gap between the hybridized molecules; c) contacting the tissue sample in (b) with nucleotide bases and ligation reagents such that the gap between the 5′ gene specific probe and 3″ gene specific probe hybridized to the mRNA transcript is filled with nucleotide bases complementary to the mRNA transcript, and a 5′ gene specific probe and a 3′ gene specific probe are ligated together to form one or more ligated gene specific probe pairs; d) removing mRNA transcripts hybridized to ligated gene specific probe pairs and leaving ligated gene specific probe pair oligonucleotide sequences; and e) capturing the ligated gene specific probe pair oligonucleotide sequences of (d) on the substrate by binding of the sequence complementary to the first universal adapter sequence in the 5′ gene specific probe to the first universal adapter sequence of the capture oligonucleotide (e.g., Rd2 adapter).

In various embodiments, the nucleotide gap is from 1-50 or more nucleotides, including 50 or more nucleotides, 1-50 nucleotides, 1-40 nucleotides, 1-30 nucleotides 1-20 nucleotides or 1-10 nucleotides.

In various embodiments, the tissue sample is a fresh tissue sample, a frozen tissue sample, or a formalin-fixed paraffin-embedded (FFPE) tissue sample.

It is contemplated that the methods further comprise indexing and sequencing the ligated gene specific probe pairs comprising f) performing extension reactions and PCR on the oligonucleotide of (e) to yield a PCR template representative of one or more mRNA transcripts in the tissue sample; g) eluting of the PCR template; and h) carrying out an indexing PCR to generate a double stranded PCR product comprising the first strand PCR product and a second strand complementary to the first strand PCR product. In various embodiments, methods further comprise sequencing the PCR product of (h) and determining the location of the mRNA transcript in the tissue based on a position of the spatial barcode (SBC) sequence.

The present disclosure provides improved methods for generating a RNA library, e.g., a mRNA library, from a tissue sample, e.g., fresh frozen or formalin-fixed paraffin embedded tissue sample, by improving efficiency of capture of mRNA transcripts from the tissue sample, thereby generating a more complete transcriptomics library.

Existing targeted ex-situ spatial approaches often involve ligating probe pairs against RNA targets within tissue. Unless gap-fill then ligation is performed, no sequence information from the RNA is obtained, rather the ligated probes are counted via sequencing. For instance, if mutations (SNVs or altered splice junctions, etc.) are present in the RNA, they would not be detected.

Multiple methods are proposed for capturing RNA with targeted probes which can then be hybridized to substrate linked probes which comprise a spatially barcoded sequence for RNA library preparation.

In one aspect, the disclosure provides a method for preparing a spatially barcoded RNA library from a tissue sample comprising, (a) contacting the tissue sample with a plurality of RNA capture probes that hybridize with RNA in the tissue sample, wherein each of the RNA capture probes comprises an RNA capture oligonucleotide sequence complementary to an RNA in the sample and a first substrate capture oligonucleotide complementary to a first domain of a plurality of splint oligonucleotides; (b) hybridizing the RNA capture oligonucleotide of the RNA capture probes with RNA in the tissue sample to form RNA-RNA capture probe hybrids; (c) carrying out extension of the RNA capture oligonucleotide of the RNA-RNA capture probe hybrids using reverse transcriptase to form a plurality of first strand cDNA molecules, wherein each of the first strand cDNA molecules comprises the RNA capture oligonucleotide and the first substrate capture oligonucleotide; (d) capturing the first strand cDNA molecules on a substrate, wherein the substrate comprises a plurality of substrate capture probes each comprising a spatial barcode and a second substrate capture oligonucleotide complementary to a second domain of the splint oligonucleotides, and wherein the capturing comprises hybridizing the splint oligonucleotides with the first substrate capture oligonucleotide of the first strand cDNA molecules and the second substrate capture oligonucleotide of the substrate capture probes; and (e) ligating the captured first strand cDNA molecules to the substrate capture probes, thereby forming spatially barcoded first strand cDNA molecules.

In various embodiments, the substrate capture probe further comprises a substrate anchor moiety.

In various embodiments, the surface oligonucleotide further comprises a P7 adapter and the RNA capture probe primer for reading the spatial barcode sequence.

Also contemplated is a method for preparing a spatially barcoded RNA library from a tissue sample comprising, (a) contacting the tissue sample with a plurality of RNA capture probes that hybridize with RNA in the tissue sample, wherein the RNA capture probes comprise an RNA capture oligonucleotide complementary to an RNA in the sample and a handle sequence; (b) hybridizing the RNA capture oligonucleotide of the RNA capture probes with RNA in the tissue sample to form RNA-RNA capture probe hybrids; (c) carrying out extension of the RNA capture oligonucleotide of the RNA-RNA capture probe hybrids using reverse transcriptase to form a plurality of first strand cDNA molecules, wherein each of the first strand cDNA molecules comprises the RNA capture oligonucleotide and the handle sequence; (d) adding a 3′ end oligonucleotide to the 3′ end of each first strand cDNA molecule, wherein the 3′ end oligonucleotide comprises a substrate capture oligonucleotide complementary to a first domain of a plurality of substrate capture probes on a substrate, wherein each of the plurality of substrate capture probes comprises, in the 5′ to 3′ orientation, a substrate anchor sequence, a spatial barcode, and the first domain; (e) hybridizing the substrate capture oligonucleotide of the first strand cDNA molecules with the first domain of the substrate capture probes; and (f) carrying out extension of the first domain of the hybridized substrate capture probes to form a plurality of spatially barcoded first strand cDNA molecules.

In various embodiments, the handle sequence is a PCR handle sequence, a molecular identifier, a UMI, or any combination thereof. In various embodiments, the handle sequence is a P5 adapter sequence.

In various embodiments, the 3′ end oligonucleotide is added by tagmentation. In various embodiments, the 3′ end oligonucleotide is added by click chemistry, or oNTP-directed adapterization. In various embodiments, the 3′OH is added by terminating the extension reaction with a click labeled nucleotide. In various embodiments, the click labeled nucleotide is an azide or alkyne labeled oligonucleotide. In various embodiments, the extension reaction adds a poly A sequence to the 3′ extended sequence.

In various embodiments, the first strand cDNA is captured with a polyT sequence on the surface capture oligonucleotide.

Further provided is a method for preparing a spatially barcoded RNA library from a tissue sample comprising, (a) contacting the tissue sample with a plurality of RNA capture probes that hybridize with RNA in the tissue sample, wherein the RNA capture probes comprise an RNA capture oligonucleotide complementary to an RNA in the sample and a handle sequence; (b) hybridizing the RNA capture oligonucleotide of the RNA capture probes with RNA in the tissue sample to form RNA-RNA capture probe hybrids; (c) carrying out extension of the RNA capture oligonucleotide of the RNA-RNA capture probe hybrids using reverse transcriptase to form a plurality of first strand cDNA molecules, wherein each of the first strand cDNA molecules comprises the RNA capture oligonucleotide and the handle sequence; (d) adding a 3′ end oligonucleotide to the 3′ end of each first strand cDNA molecule, via template switching, comprising contacting the first strand cDNA molecule with a reverse transcriptase (RT) and a template switch oligonucleotide (TSO), wherein the RT incorporates untemplated cytosine nucleotides at the 3′ end of the first cDNA and the TSO comprises a sequence capable of hybridizing to the untemplated cytosine nucleotides, wherein the 3′ end oligonucleotide is appended to the 3′ end of the first cDNA and the RT extends to generate a TSO complement; wherein the 3′ end oligonucleotide comprises a substrate capture oligonucleotide complementary to a first domain of a plurality of substrate capture probes on a substrate, wherein each of the plurality of substrate capture probes comprises, in the 5′ to 3′ orientation, a substrate anchor sequence, a spatial barcode, and the first domain; (e) hybridizing the substrate capture oligonucleotide of the first strand cDNA molecules with the first domain of the substrate capture probes; and (f) carrying out extension of the first domain of the hybridized substrate capture probes to form a plurality of spatially barcoded first strand cDNA molecules.

In various embodiments, the substrate capture probes are released from the substrate prior to hybridizing the substrate capture oligonucleotide with the first domain of the substrate capture probe.

In various embodiments, the first domain is a poly T sequence.

In another aspect, the disclosure provides a method for preparing a spatially barcoded RNA library from a tissue sample comprising, (a) contacting the tissue sample with a plurality of RNA capture probes that hybridize with RNA in the tissue sample, wherein the RNA capture probes comprise an RNA capture oligonucleotide complementary to an RNA in the sample and a handle sequence; (b) hybridizing the RNA capture oligonucleotide of the RNA capture probes with RNA in the tissue sample to form RNA-RNA capture probe hybrids; (c) carrying out extension of the RNA capture oligonucleotide of the RNA-RNA capture probe hybrids using reverse transcriptase to form a plurality of first strand cDNA molecules, wherein each of the first strand cDNA molecules comprises the RNA capture oligonucleotide and the handle sequence; (d) adding a 3′ end oligonucleotide to the 3′ end of each first strand cDNA molecule, via template switching, comprising contacting the first strand cDNA molecule with a reverse transcriptase (RT) and a template switch oligonucleotide (TSO), wherein the RT incorporates untemplated cytosine nucleotides at the 3′ end of the first cDNA and the TSO comprises a sequence capable of hybridizing to the untemplated cytosine nucleotides, wherein the 3′ end oligonucleotide is appended to the 3′ end of the first cDNA and the RT extends to generate a TSO complement; wherein the 3′ end oligonucleotide comprises a substrate capture oligonucleotide complementary to a first domain of a plurality of substrate capture probes on a substrate, wherein each of the plurality of substrate capture probes comprises, in the 5′ to 3′ orientation, a substrate anchor sequence, a second handle, a spatial barcode, and the first domain; (e) releasing the substrate capture probes from the substrate; (f) hybridizing the substrate capture oligonucleotide of the first strand cDNA molecules with the first domain of the substrate capture probes; and (g) contacting the first strand with a second strand synthesis mix comprising a TSO primer and extending the TSO primer using the first strand as a template to generate a second strand complementary to the first strand, the second strand comprising the TSO, a second cDNA complementary to the first cDNA, and second strand barcode information comprising a spatial bar-code sequence complement (SBC′) that is complementary to the spatial barcode sequence (SBC).

In various embodiments, the first domain is a poly G sequence that hybridizes with the poly C sequence on the TSO. In various embodiments, the handle is a P5 sequence and the second handle is a P7 sequence.

In another aspect, the disclosure provides a method for preparing a spatially barcoded RNA library from a tissue sample comprising, (a) contacting the tissue sample with a plurality of RNA capture probes that bind RNA in the tissue sample, wherein each of the RNA capture probes comprise a RNA capture oligonucleotide complementary to an RNA in the sample and a substrate capture oligonucleotide complementary to a first domain of a plurality of substrate capture probes on a substrate, wherein the RNA capture oligonucleotide complementary to the RNA is blocked on the 3′ end; wherein each of the substrate capture probes comprises, in the 5′ to 3′ orientation, the first domain and a first substrate anchor sequence and is in proximity to one or more barcoded substrate probes on the substrate, and wherein each of the barcoded substrate probes comprises, in the 5′ to 3′ orientation, a second substrate anchor sequence, a spatial barcode, and a random priming sequence; (b) hybridizing the RNA capture oligonucleotide of the RNA capture probes with RNA in the tissue sample to form RNA-RNA capture probe hybrids having a 5′ single-stranded RNA region; (c) hybridizing the substrate capture oligonucleotide of the RNA-RNA capture probe hybrids with the first domain of the substrate capture probes; (d) hybridizing the 5′ single-stranded RNA region of the RNA-RNA capture probe hybrids with the random priming sequence of the barcoded substrate probes; and (e) carrying out extension of the random priming sequences hybridized to the 5′ single-stranded RNA regions using reverse transcriptase to form a plurality of spatially barcoded first strand cDNA molecules.

In various embodiments, the nucleotide sequence complementary to RNA in the sample is a polyT oligonucleotide, a randomer, a semi-randomer, or a target specific sequence. In various embodiments, the nucleotide sequence complementary to an RNA in the sample is a polyT oligonucleotide.

In various embodiments, the methods further comprise a step of removing RNA from the sample. In various embodiments, the RNA is removed from the sample after the extension to form first strand cDNA. In various embodiments, the RNA is removed by enzymatic or thermal methods.

Also provided is a method for preparing a spatially barcoded RNA library from a tissue sample comprising, (a) contacting the tissue sample with a plurality of RNA capture probes that hybridize with RNA in the tissue sample, wherein each of the RNA capture probes comprises an RNA capture oligonucleotide complementary to an RNA in the sample and a substrate capture oligonucleotide complementary to a first domain of a plurality of substrate capture probes on a substrate, wherein each of the substrate capture probes comprises, in the 5′ to 3′ orientation, a substrate anchor sequence, the first domain, a linker, a spatial barcode, and a random priming sequence; (b) hybridizing the RNA capture probes with the RNA in the tissue sample to form RNA-RNA capture probe hybrids having a 5′ single-stranded RNA region; (c) hybridizing the substrate capture oligonucleotide of the RNA-RNA capture probe hybrids with the first domain of the substrate capture probes; (d) hybridizing the 5′ single-stranded RNA regions of the RNA-RNA capture probe hybrids with the random priming sequence of the substrate capture probes; and (e) carrying out extension of the random priming sequences hybridized to the 5′ single-stranded RNA regions using reverse transcriptase to form a plurality of spatially barcoded first strand cDNA molecules.

In various embodiments, the linker is a linker that cannot be read through by a polymerase.

In another aspect, the disclosure contemplates a method for preparing a spatially barcoded RNA library from a tissue sample comprising, (a) contacting the tissue sample with a plurality of RNA capture probes that bind RNA in the tissue sample, wherein each of the RNA capture probes comprise an RNA capture oligonucleotide complementary to an RNA in the sample and a substrate capture oligonucleotide complementary to a first domain of a plurality of substrate capture probes on a substrate; wherein each of the substrate capture probes comprises, in the 5′ to 3′ orientation, the first domain and a first substrate anchor sequence and is in proximity to at least one of a plurality of barcoded substrate probes on the substrate, and wherein each barcoded substrate probe comprises, in the 5′ to 3′ orientation, a spatial barcode and a second substrate anchor sequence; (b) hybridizing the RNA capture oligonucleotide of the RNA capture probes with RNA in the tissue sample to form RNA-RNA-capture probe hybrids; (c) capturing the RNA-RNA capture probe hybrids on the substrate by hybridizing substrate capture oligonucleotide of the RNA-RNA capture probe hybrids with the first domain of the substrate capture probes; (d) carrying out extension of the RNA capture oligonucleotide of the captured RNA-RNA capture probe hybrids using reverse transcriptase to form a plurality of first strand cDNA molecules; and (e) ligating each of the first strand cDNA molecules to the proximal barcoded substrate probe, thereby forming spatially barcoded first strand cDNA molecules.

Further contemplated is a method for preparing a spatially barcoded RNA library from a tissue sample comprising, (a) contacting the tissue sample with a plurality of RNA capture probes that bind RNA in the tissue sample, wherein each of the RNA capture probes comprise an RNA capture oligonucleotide complementary to an RNA in the sample and a substrate capture oligonucleotide complementary to a first domain of a plurality of substrate capture probes on a substrate, wherein the RNA capture oligonucleotide complementary to the RNA is blocked on the 3′ end; wherein each of the substrate capture probes comprises, in the 5′ to 3′ orientation, the first domain and a first substrate anchor sequence and is in proximity to at least one of a plurality of barcoded substrate probes on the substrate, and wherein each barcoded substrate probe comprises, in the 5′ to 3′ orientation, a polyT sequence, a spatial barcode and a second substrate anchor sequence; (b) hybridizing the RNA capture oligonucleotide of the RNA capture probes with RNA in the tissue sample to form RNA-RNA-capture probe hybrids; (c) capturing the RNA-RNA capture probe hybrids on the substrate by hybridizing substrate capture oligonucleotide of the RNA-RNA capture probe hybrids with the first domain of the substrate capture probe; (d) polyadenylating the RNA in the sample at the 3′ end; and (e) carrying out extension of the RNA capture oligonucleotide of the captured RNA-RNA capture probe hybrids using reverse transcriptase to form a plurality of first strand cDNA molecules.

In various embodiments, the polyadenylation is carried out using polyA polymerase.

Also provided is a method for preparing a spatially barcoded RNA library from a tissue sample comprising, (a) contacting the tissue sample with a plurality of RNA capture probes that hybridize with RNA in the tissue sample, wherein each of the RNA capture probes has a hairpin structure and comprises an DNA capture oligonucleotide complementary to RNA in the sample and a substrate capture oligonucleotide complementary to a first domain of a plurality of substrate capture probes on a substrate, wherein the DNA capture oligonucleotide of the RNA capture probes comprises a single stranded region, and wherein each of the substrate capture probes comprises, in the 5′ to 3′ orientation, a substrate anchor sequence, a spatial barcode, the first domain, and a second domain, wherein the second domain comprises at least one RNA nucleotide or nucleoside; (b) hybridizing the RNA capture probes with the RNA in the tissue sample to form RNA-RNA capture probe hybrids, wherein each of the RNA-RNA capture probe hybrids comprises a 5′ single-stranded RNA end region; (c) capturing the substrate capture oligonucleotide of the RNA-RNA capture probe hybrids on the substrate by hybridizing the substrate capture oligonucleotide of the RNA-RNA capture probe hybrids with the first domain of the substrate capture probes; (d) phosphorylating the 5′ single-stranded RNA end region of the captured RNA-RNA capture probe hybrids and contacting the captured RNA-RNA capture probe hybrids with a 5′ to 3′ riboexonuclease to digest the phosphorylated 5′ single-stranded RNA end region; and (e) ligating the digested 5′ RNA end region of the captured RNA-RNA capture probe hybrids to the second domain of the substrate capture probes to form a plurality of DNA-RNA chimeras on the substrate.

In various embodiments, the ligating is carried out with T4 ligase.

In various embodiments, the RNA of the captured RNA-RNA capture probe hybrids is 5′ phosphorylated prior to ligation.

In various embodiments, the methods further comprise generating first strand cDNA from the plurality of DNA-RNA chimeras on the substrate. In various embodiments, the first strand cDNAs can be hybridized from the surface and processed for sequencing.

In various embodiments, the reverse transcription is carried out using a DNA random primer, optionally which comprises a P5 adaptor.

In various embodiments, the cDNA extension templates can be de-hybridized from the RNA in the tissue by chemical, enzymatic, or thermal de-hybridization. In various embodiments, the cDNA extension templates can be de-hybridized from the RNA on a substrate by chemical, enzymatic, or thermal de-hybridization. In various embodiments, the de-hybridization step occurs before or after the capturing step.

In various embodiments, the RNA capture probe is selected from the group consisting of a poly-T sequence, a poly-U sequence, a randomer, a semi-random sequence, or a target-specific probe. In various embodiments, the RNA capture probe is a poly-T sequence.

In various embodiments, the RNA capture probe comprises at least 10 deoxythymidine residues. In various embodiments, the RNA capture probes comprise a plurality of different target-specific RNA capture probe sequences. In various embodiments, the RNA capture probes comprise at least 10 nucleotides complementary to a nucleotide sequence of a target RNA. In various embodiments, the RNA capture probe or surface capture probe is between 8 to 80 nucleotides. In various embodiments, the RNA capture probe is between 8-80 nucleotides or between 10-50 nucleotides.

In various embodiments, the tissue sample is permeabilized prior to contacting the tissue sample with a plurality of RNA capture probes. In various embodiments, the tissue sample is treated with one or more blocking reagents prior to contacting the tissue sample with a plurality of RNA capture probes. In various embodiments, the tissue sample is permeabilized and treated with one or more blocking reagents prior to contacting the tissue sample with a plurality of RNA capture probes.

In various embodiments, the substrate is a bead, a bead array, a spotted array, a substrate comprising a plurality of wells, a flow cell, clustered particles arranged on a surface of a chip, a film, or a plate. In various embodiments, the substrate comprises a plurality of nanowells or microwells.

In various embodiments, the tissue sample is a fresh tissue sample, a frozen tissue sample, or a formalin-fixed paraffin-embedded (FFPE) tissue sample. In various embodiments, when the sample is a FFPE sample the methods can further comprise decrosslinking the FFPE sample, optionally wherein the decrosslinking is carried out using TE buffer, pH 9.

In various embodiments, the methods further comprise determining the spatial location of one or more of the spatially barcoded first strand cDNA molecules or copies thereof by correlating the spatial barcode sequences of the spatially barcoded first strand cDNA molecules or copies thereof with the spatial locations of the surface oligonucleotide molecules on the substrate containing corresponding spatial barcode sequences.

In various embodiments, the methods further comprise recovering the spatially barcoded first strand cDNA molecules and amplifying them to generate cDNA libraries.

In various embodiments, the spatially barcoded first strand cDNA molecules are recovered by contacting the spatially barcoded first strand cDNAs on the substrate with a DNA polymerase and one or more primers to generate spatially barcoded second strand cDNAs complementary to the spatially barcoded first strand cDNAs and removing the spatially barcoded second strand cDNAs from the substrate.

In various embodiments, the one or more primers each comprise a random priming sequence. In various embodiments, the random priming sequences comprises nine random nucleotides.

In various embodiments, the spatially barcoded second strand cDNAs each comprise a unique molecular identifier (UMI), wherein the UMI comprises an intrinsic sequence and an extrinsic sequence, wherein the extrinsic sequence is a sequence complementary to the random priming sequence used to generate the second strand cDNA, and wherein the intrinsic sequence is a sequence complementary to the first strand cDNA template sequence used to generate the second strand cDNA.

In various embodiments, the one or more primers each comprise a molecular identifier barcode. In various embodiments, the one or more primers each comprise a UMI barcode.

In various embodiments, the spatially barcoded second strand cDNAs are removed from the substrate by chemical or physical dehybridization.

In various embodiments, the anchor sequence comprises a cleavage site, and hybrids of the spatially barcoded first and second strand cDNAs are removed from the substrate by enzymatic cleavage at the cleavage site. In various embodiments, the cleavage site is a binding site for a restriction endonuclease. In various embodiments, the anchor sequence comprises a cleavage site, and wherein the spatially barcoded first strand cDNA molecules are recovered by enzymatic cleavage at the cleavage site. In various embodiments, the cleavage site is a binding site for a restriction endonuclease.

In various embodiments, the methods further comprise sequencing at least a portion of the cDNA libraries to determine the spatial barcode sequence for each molecule.

In various embodiments, the methods further comprise determining the spatial location of one or more cDNA molecules by correlating the spatial barcode sequences of the one or more cDNA molecules with the spatial locations of the surface oligonucleotide molecules on the substrate containing corresponding spatial barcode sequences.

In various embodiments, the methods further comprise indexing and sequencing spatially barcoded first strand cDNAs, the method comprising, performing extension reactions and PCR on the spatially barcoded first strand cDNAs to yield a PCR template comprising a first strand PCR product representative of one or more RNA transcripts in the tissue sample; eluting the PCR template; carrying out an indexing PCR to generate a double stranded PCR product comprising the first strand PCR product and a second strand complementary to the first strand PCR product.

In various embodiments, the methods further comprise sequencing the PCR product and determining the location of the RNA transcript in the tissue based on the spatial barcode of first strand cDNA.

In various embodiments, the double stranded PCR product comprises a second clustering sequence on the second strand complementary to the first strand PCR product and, optionally, an index sequence.

In various embodiments, the PCR products are further processed by tagmentation to generate a spatial transcriptomics library. In various embodiments, the tagmentation comprises on substrate tagmentation. In some embodiments, the tagmentation comprises on bead tagmentation, wherein the bead comprises a plurality of bead-linked transposomes (BLT). In some embodiments, the BLT comprises i) a plurality of oligonucleotides comprising a first clustering sequence (P7), a first index sequence and a Read 1 sequencing primer (Rd1 SP) and ii) a plurality of oligonucleotides comprising a second clustering sequence (P5), a second index sequence and a Read 2 sequencing primer (Rd2 SP).

In various embodiments, the RNA library is an mRNA library.

In various embodiments, the methods determine RNA expression in a single cell with the tissue sample. In various embodiments, the methods determine RNA expression in one or more subcellular components in the single cell. In various embodiments, the subcellular component is a cell nucleus, cytoplasm, or mitochondria.

In various embodiments, the substrate or surface of the substrate comprises a material selected from glass, silicon, poly-L-lysine coated materials, nitrocellulose, polystyrene, cyclic olefin copolymers (COCs), cyclic olefin polymers (COPs), polyacrylamide, polypropylene, polyethylene, or polycarbonate.

Also provided is a method of identifying a genetic variation in a subject having or at risk of having a disease comprising generating a sample RNA library, e.g., mRNA library, from a tissue sample from the subject according to the methods described herein, comparing the genetic information from the sample RNA library, e.g., mRNA library, to a control RNA library, e.g., mRNA library, or to a sample of the subject prior to disease, and identifying a genetic variation in the sample RNA library, e.g., mRNA library, associated with the disease. Optionally, the method comprises treatment of the subject with a therapy specific for the disease.

In various embodiments, the disease is a genetic defect, cancer, an autoimmune disease, a metabolic disorder or other disease described herein. Additional diseases or conditions are described in more detail in the Detailed Description.

It is understood that each feature or embodiment, or combination, described herein is a non-limiting, illustrative example of any of the aspects of the invention and, as such, is meant to be combinable with any other feature or embodiment, or combination, described herein. For example, where features are described with language such as “one embodiment”, “various embodiments”, “some embodiments”, “certain embodiments”, “further embodiment”, “specific exemplary embodiments”, and/or “another embodiment”, each of these types of embodiments is a non-limiting example of a feature that is intended to be combined with any other feature, or combination of features, described herein without having to list every possible combination.

Such features or combinations of features apply to any of the aspects of the invention. Where examples of values falling within ranges are disclosed, any of these examples are contemplated as possible endpoints of a range, any and all numeric values between such endpoints are contemplated, and any and all combinations of upper and lower endpoints are envisioned.

### BRIEF DESCRIPTION OF THE DRAWINGS

FIG. 1 is a schematic diagram of a method of capturing mRNA transcripts from a tissue sample in situ using capture probes.

FIG. 2 is a schematic diagram of a method of capturing mRNA transcripts from a tissue sample in situ using capture probes, wherein the capture probes hybridized to the transcripts result in a nucleotide gap between the hybridized sequences.

FIG. 3. Schematic of an exemplary RNA library preparation workflow as described herein.

FIG. 4A-4D. Schematic of an alternate RNA library preparation workflow as described herein. FIG. 4A shows the general workflow, while methods of adding a 3′ oligonucleotide are added by oNTP-directed adapterization or click chemistry (FIG. 4B), template switching (FIG. 4C) or template switching, where the template switching primer is released from the substrate and is spatially barcoded (FIG. 4D).

FIG. 5A-5B. Schematics of an alternate RNA library preparation workflow as described herein using a 3′ blocked oligonucleotide on the target probe.

FIG. 6A-6B. Schematic of an alternate RNA library preparation workflow as described herein.

FIG. 7. Schematic of an alternate RNA library preparation workflow as described herein using a hairpin probe.

FIG. 8 illustrates a workflow based on the scheme in FIG. 3.

FIG. 9 illustrates a workflow based on the scheme in FIGS. 4A and 4B.

FIG. 10 illustrates a workflow based on the scheme in FIG. 4C.

FIG. 11 illustrates a workflow based on the scheme in FIG. 4D.

FIG. 12 illustrates a workflow based on the scheme in FIG. 5A.

FIG. 13 illustrates a workflow based on the scheme in FIG. 5B.

FIG. 14 illustrates a workflow based on the scheme in FIG. 6A.

FIG. 15 illustrates a workflow based on the scheme in FIG. 6B.

FIG. 16 illustrates a workflow based on the scheme in FIG. 7.

### DETAILED DESCRIPTION

In order to overcome the technical limitations of isolating mRNA transcripts from fresh frozen or FFPE tissue sample, described herein are in situ methods to capture and generate spatially-barcoded libraries from such compromised tissue mRNAs.

Described herein are a variety of methods and compositions that allow for the characterization of a genetic profile in tissues while preserving spatial information related to the origin of target gene or polynucleotide in the tissue. In various embodiments, the method includes a substrate on which a plurality of capture probes are immobilized such that each capture probe occupies a distinct position on the array. Each capture probe includes, among other sequences and/or molecules, a unique positional nucleic acid tag (i.e., a spatial address or indexing sequence). Each spatial address corresponds to the position of the capture probe on the array. The position of the capture probe on the array may be correlated with a position in the tissue sample.

Examples of a gene or polynucleotide in a tissue sample include genomic DNA, methylated DNA, specific methylated DNA sequences, messenger RNA (mRNA), polyA mRNA, fragmented mRNA, fragmented DNA, mitochondrial DNA, ribosomal RNA (rRNA), viral RNA, microRNA, in situ synthesized PCR products, and RNA/DNA hybrids. Also contemplated are non-coding RNA (ncRNA), small nucleolar RNA (snoRNA), and/or small nuclear RNA (snRNA).

A nucleic acid tag encoding location (i.e., a spatial address or indexing sequence) can be coupled to a nucleic acid capture region or any other molecule that binds a target gene or polynucleotide. Examples of other molecules that may be coupled to a nucleic acid tag include antibodies, antigen binding domains, proteins, peptides, receptors, haptens, etc.

Described herein are a variety of methods and compositions that allow for the characterization of transcriptomes and/or genomic variation in tissues while preserving spatial information related to the origin of target nucleic acids in the tissue. For example, the methods disclosed herein can enable the identification of the location of a cell or a cell cluster in a tissue biopsy that carries an aberrant mutation. The methods provided herein can therefore be useful for diagnostic purposes, e.g., for the diagnosis of cancer, and possibly aid in the selection of targeted therapies.

The present disclosure is based, in part, on the recognition that information related to the spatial origin of a nucleic acid in a tissue sample can be encoded in the nucleic acid in the process of preparing the nucleic acid for sequencing. For example, nucleic acids from a tissue sample can be tagged by probes including location-specific sequence information (a “spatial address”). Spatially addressed nucleic acid molecules from a tissue sample can then be sequenced in bulk. The sequence-identical nucleic acid molecules originating from different regions in a tissue sample can be distinguished based on their spatial address and can be mapped onto their regions of origin in the tissue sample. Additionally, spatial addressing of nucleic acids could increase the sensitivity of detection of single nucleotide variations (SNVs) or single nucleotide polymorphisms (SNPs) in a tissue sample.

In some methods described herein probes for spatial tagging include, e.g., combinations of spatial address regions and gene-specific capture regions. The spatially addressed and gene-specific probes can be contacted with the tissue sample as immobilized probes on a capture array.

The present disclosure recognizes that spatial addressing of nucleic acids from a tissue sample can involve two-dimensional spatial addressing, e.g., to correlate the position of a nucleic acid on a two-dimensional capture array with the position of the nucleic acid in a two-dimensional tissue section. Spatial addressing can be performed also in additional dimensions. For example, spatial address sequences can be added to nucleic acids to describe the relative spatial position of a nucleic acid in a third or fourth dimension, e.g., by describing the position of a tissue section in a tissue biopsy, or the position of a tissue biopsy in a subject's organ. Temporal address sequences could be added to nucleic acids from a tissue sample to denote a timepoint in a timecourse experiment, e.g., inquiring into changes of gene-expression in a cell in response to a physical or chemical stimulus, such as a drug treatment during a clinical trial.

### Definitions

Unless otherwise stated, the following terms used in this application, including the specification and claims, have the definitions given below.

As used in this specification and the appended claims, the singular forms “a”, “an” and “the” include plural referents unless the content clearly dictates otherwise. Thus, for example, reference to “a capture probe” includes a mixture of two or more capture probes, and the like.

The term “about,” particularly in reference to a given quantity, is meant to encompass deviations of plus or minus five percent.

As used herein, the terms “includes,” “including,” “includes,” “including,” “contains,” “containing,” and any variations thereof, are intended to cover a non-exclusive inclusion, such that a process, method, product-by-process, or composition of matter that includes, includes, or contains an element or list of elements does not include only those elements but can include other elements not expressly listed or inherent to such process, method, product-by-process, or composition of matter.

As used herein an “anchor” refers to a moiety that attaches a nano-scaffold to a substrate. An anchor includes a chemical moiety, peptide, or oligonucleotide. A polynucleotide anchor may be between 4-20 nucleotides.

As used herein a “splint oligonucleotide” refers to an oligonucleotide comprising a sequence complementary to a region on a surface probe on a nanostructure and another sequence complementary to a surface oligonucleotide, e.g., attached to a substrate. In various embodiments, the splint oligonucleotide is between 10-25 nucleotides or between 15-25 nucleotides. In various embodiments, the splint oligonucleotide is 20 nucleotides. In various embodiment, the splint oligonucleotide is 15, 16, 17, 18, 9, 20, 21, 22, 23, 24, or 25, nucleotides.

As used herein a “surface oligonucleotide” refers to an oligonucleotide comprising an anchor sequence for attaching the oligo to the surface of a substrate, a spatial barcode sequence and a sequence that hybridizes with a splint oligonucleotide. In various embodiments, the surface oligonucleotide is between 15-25 nucleotides. In various embodiments, the surface oligonucleotide is greater than 20 nucleotides. In various embodiment, the surface oligonucleotide is 15, 16, 17, 18, 9, 20, 21, 22, 23, 24, 25, 26, 27, 28, 29 or 30 nucleotides or more.

As used herein, the terms “address,” “tag,” or “index,” when used in reference to a nucleotide sequence is intended to mean a unique nucleotide sequence that is distinguishable from other indices as well as from other nucleotide sequences within polynucleotides contained within a sample. A nucleotide “address,” “tag,” or “index” can be a random or a specifically designed nucleotide sequence. An “address,” “tag,” or “index” can be of any desired sequence length so long as it is of sufficient length to be unique nucleotide sequence within a plurality of indices in a population and/or within a plurality of polynucleotides that are being analyzed or interrogated. A nucleotide “address,” “tag,” or “index” of the disclosure is useful, for example, to be attached to a target polynucleotide to tag or mark a particular species for identifying all members of the tagged species within a population. Accordingly, an index is useful as a barcode where different members of the same molecular species can contain the same index and where different species within a population of different polynucleotides can have different indices.

As used herein, the terms “address,” “tag,” “index,” or “barcode” when used in reference to a nucleotide sequence is intended to mean a unique nucleotide sequence that is distinguishable from other indices as well as from other nucleotide sequences within polynucleotides contained within a sample. A nucleotide “address,” “tag,” “index” or “barcode” can be a random or a specifically designed nucleotide sequence. An “address,” “tag,” “index” or “barcode” can be of any desired sequence length so long as it is of sufficient length to be unique nucleotide sequence within a plurality of indices in a population and/or within a plurality of polynucleotides that are being analyzed or interrogated. A nucleotide “address,” “tag,” “index” or “barcode” of the disclosure is useful, for example, to be attached to a target polynucleotide to tag or mark a particular species for identifying all members of the tagged species within a population. Accordingly, an index is useful as a barcode where different members of the same molecular species can contain the same index and where different species within a population of different polynucleotides can have different indices.

A tag/index/barcode sequence can be unique to a single nucleic acid species in a population or can be shared by several different nucleic acid species in a population. For example, each nucleic acid probe in a population can include different tag/index/barcode sequences from all other nucleic acid probes in the population. Alternatively, each nucleic acid probe in a population can include different tag/index/barcode sequences from some or most other nucleic acid probes in a population. For example, each probe in a population can have a tag/index/barcode that is present for several different probes in the population even though the probes with the common tag/index/barcode differ from each other at other sequence regions along their length. In particular embodiments, one or more tag/index/barcode sequences that are used with a biological specimen are not present in the genome, transcriptome or other nucleic acids of the biological specimen. For example, tag/index/barcode sequences can have less than 80%, 70%, 60%, 50% or 40% sequence identity to the nucleic acid sequences in a particular biological specimen.

As used herein, a “spatial address,” “spatial tag”, “spatial barcode”, “barcode sequence” or “spatial index,” when used in reference to a nucleotide sequence, means an address, tag, barcode or index encoding spatial information related to the region or location of origin of an addressed, tagged, barcoded, or indexed nucleic acid in a tissue sample. The sequence can be a naturally occurring sequence or a sequence that does not occur naturally in the organism from which the barcoded nucleic acid was obtained.

As used herein, the term “substrate” is intended to mean a solid support or support structure. The term includes any material that can serve as a solid or semi-solid foundation for creation of features such as wells for the deposition of biopolymers, including nucleic acids, polypeptide and/or other polymers. Non-limiting examples of substrates include a bead array, a spotted array, clustered particles arranged on a surface of a chip, a film, a multi-well plate, and a flow cell. A substrate as provided herein is modified, for example, or can be modified to accommodate attachment of biopolymers by a variety of methods well known to those skilled in the art. Exemplary types of substrate materials include glass, modified glass, functionalized glass, inorganic glasses, microspheres, including inert and/or magnetic particles, plastics, polysaccharides, nylon, nitrocellulose, ceramics, resins, silica, silica-based materials, carbon, metals, an optical fiber or optical fiber bundles, a variety of polymers other than those exemplified above and multiwell microtiter plates. Specific types of exemplary plastics include acrylics, polystyrene, copolymers of styrene and other materials, polypropylene, polyethylene, polybutylene, polyurethanes and TEFLON™. Specific types of exemplary silica-based materials include silicon and various forms of modified silicon.

Those skilled in the art will know or understand that the composition and geometry of a substrate as provided herein can vary depending on the intended use and preferences of the user. Therefore, although planar substrates such as slides, chips wafers or beads are useful for microarrays, those skilled in the art will understand that a wide variety of other substrates exemplified herein or well known in the art also can be used in the methods and/or compositions herein.

In some embodiments, the solid support comprises one or more surfaces that are accessible to contact with reagents, beads, or analytes. The surface can be substantially flat or planar. Alternatively, the surface can be rounded or contoured. Example contours that can be included on a surface are wells (e.g., microwells or nanowells), depressions, pillars, ridges, channels or the like. Example materials that can be used as a surface include glass such as modified or functionalized glass; plastic such as acrylic, polystyrene or a copolymer of styrene and another material, polypropylene, polyethylene, polybutylene, polyurethane or TEFLON™; polysaccharides or cross-linked polysaccharides such as agarose or Sepharose; nylon; nitrocellulose; resin; silica or silica-based materials including silicon and modified silicon, carbon-fiber; metal; inorganic glass; optical fiber bundle, or a variety of other polymers. A single material or mixture of several different materials can form a surface useful in certain examples. In some examples, a surface comprises wells (e.g., microwells or nanowells). In some aspects, the surface comprises wells in an array of wells (e.g., microwells or nanowells) on glass, silicon, plastic or other suitable solid supports with patterned, covalently-linked gel such as poly(N-(5-azidoacetamidylpentyl) acrylamide-coacrylamide) (PAZAM, see, for example, U.S. Pat. App. Pub. No. 2014/0079923 A1, which is incorporated herein by reference). In some examples, a support structure can include one or more layers.

Non-limiting examples of a surface include a bead array, a spotted array, clustered particles arranged on a surface of a chip, a film, a multi-well plate, and a flow cell.

In some embodiments, the solid support comprises one or more surfaces of a flowcell. The term “flowcell” as used herein refers to a chamber comprising a solid surface across which one or more fluid reagents can be flowed. The flow cell can be an ordered or random flow cell. Examples of flowcells and related fluidic systems and detection platforms that can be readily used in the methods of the present disclosure are described, for example, in Bentley et al., Nature 456:53-59 (2008), WO04/018497; U.S. Pat. No. 7,057,026; WO 91/06678; WO 07/123744; U.S. Pat. Nos. 7,329,492; 7,211,414; 7,315,019; 7,405,281, and US 2008/0108082, each of which is incorporated herein by reference.

In some embodiments, the solid support includes a patterned surface. A “patterned surface” refers to an arrangement of different regions in or on an exposed layer of a solid support. For example, one or more of the regions can be features where one or more amplification primers are present. The features can be separated by interstitial regions where amplification primers are not present. In some embodiments, the pattern can be an x-y format of features that are in rows and columns. In some embodiments, the pattern can be a repeating arrangement of features and/or interstitial regions. In some embodiments, the pattern can be a random arrangement of features and/or interstitial regions. Exemplary patterned surfaces that can be used in the methods and compositions set forth herein are described in U.S. Ser. No. 13/661,524 or US Pat. App. Publ. No. 2012/0316086, or International Patent Publication WO 2017/019456, each of which is incorporated herein by reference.

As used herein, the term “immobilized” when used in reference to a nucleic acid is intended to mean direct or indirect attachment to a solid support via covalent or non-covalent bond(s). Immobilized also refers to the state of two things being joined, fastened, adhered, attached, connected, or bound to each other. For example, an analyte, such as a nucleic acid, can be immobilized on a material, such as a bead, gel, or surface, by a covalent or non-covalent bond. In certain embodiments, covalent attachment can be used, but all that is required is that the nucleic acids remain stationary or attached to a support under conditions in which it is intended to use the support, for example, in applications requiring nucleic acid amplification and/or sequencing. Oligonucleotides to be used as capture primers or amplification primers can be immobilized such that a 3′-end is available for enzymatic extension and at least a portion of the sequence is capable of hybridizing to a complementary sequence.

Immobilization can occur via hybridization to a surface attached oligonucleotide, in which case the immobilized oligonucleotide or polynucleotide can be in the 3′-5′ orientation. Alternatively, immobilization can occur by means other than base-pairing hybridization, such as the covalent attachment set forth above

Exemplary covalent linkages include, for example, those that result from the use of click chemistry techniques. Exemplary non-covalent linkages include, but are not limited to, non-specific interactions (e.g., hydrogen bonding, ionic bonding, van der Waals interactions etc.) or specific interactions (e.g., affinity interactions, receptor-ligand interactions, antibody-epitope interactions, avidin-biotin interactions, streptavidin-biotin interactions, lectin-carbohydrate interactions, etc.). Exemplary linkages are set forth in U.S. Pat. Nos. 6,737,236; 7,259,258; 7,375,234 and 7,427,678; and US Pat. Pub. No. 2011/0059865 A1, each of which is incorporated herein by reference.

As used herein, the term “array” refers to a population of sites that can be differentiated from each other according to relative location. Different molecules that are at different sites of an array can be differentiated from each other according to the locations of the sites in the array. An individual site of an array can include one or more molecules of a particular type. For example, a site can include a single target nucleic acid molecule having a particular sequence or a site can include several nucleic acid molecules having the same sequence (and/or complementary sequence, thereof). The sites of an array can be different features located on the same substrate. Exemplary features include without limitation, wells in a substrate, beads (or other particles) in or on a substrate, projections from a substrate, ridges on a substrate or channels in a substrate. The sites of an array can be separate substrates each bearing a different molecule. Different molecules attached to separate substrates can be identified according to the locations of the substrates on a surface to which the substrates are associated or according to the locations of the substrates in a liquid or gel. Exemplary arrays in which separate substrates are located on a surface include, without limitation, those having beads in wells.

As used herein, the term “single molecular identifier” or “SMI” refers to a molecular tag, either random, non-random, or semi-random, that may be attached to a nucleic acid. In various embodiments, a SMI is a unique molecular identifier (UMI). When incorporated into a nucleic acid, a SMI can be used to correct for subsequent amplification bias by directly counting single molecular identifiers (SMIs) that are sequenced after amplification. A SMI (e.g., a UMI) can be attached to similar nucleic acids, e.g., adapters, making each nucleic acid unique. SMIs (e.g., UMIs) may also be used to uniquely tag individual molecules (e.g., individual mRNA molecules) in a sample (e.g., individual mRNA molecules in a tissue sample, cell sample, or sample library).

As used herein “unique molecular index”, “unique molecular identifier” or “UMI”, when used in reference to a capture probe or other nucleic acid is intended to refer to a portion of a probe useful as a molecular barcode to uniquely tag each molecule in a sample library. A UMI may be denoted as “NNNN . . . ” in a string of nucleic acids to designate that portion of the oligonucleotide as the UMI. A UMI may be from 6 to 20 nucleotides or more in length. In some aspects, the UMI comprises a spatial barcode.

As used herein, the term “universal sequence” refers to a series of nucleotides that is common to two or more nucleic acid molecules even if the molecules also have regions of sequence that differ from each other. A universal sequence that is present in different members of a collection of molecules can allow capture of multiple different nucleic acids using a population of universal capture nucleic acids that are complementary to the universal sequence. Similarly, a universal sequence present in different members of a collection of molecules can allow the replication or amplification of multiple different nucleic acids using a population of universal primers that are complementary to the universal sequence. Thus, a universal capture nucleic acid or a universal primer includes a sequence that can hybridize specifically to a universal sequence. Target nucleic acid molecules may be modified to attach universal adapters, for example, at one or both ends of the different target sequences. Universal capture oligonucleotides are applicable for interrogating a plurality of different oligonucleotides without necessarily distinguishing the different species whereas target-specific capture sequences are applicable for distinguishing the different species. A non-limiting example of a universal sequence is a polyT nucleotide sequence.

As used herein, a “semi-random” nucleotide sequence comprises or consists of a partially pre-determined nucleotide sequence combined with a random nucleotide sequence.

As used herein, the term “adapter” refers generally to any linear nucleic acid molecule that can be added (e.g., through synthesis or ligation) to an oligonucleotide of the disclosure. In some embodiments, adapters are copied onto the library molecules using templated polymerase synthesis (e.g., second strand cDNA synthesis as described herein). In some embodiments, adapters are ligated to a first complementary strand of the disclosure. In some embodiments, oligonucleotides of the disclosure comprise adapters (“adapter oligonucleotides”). In some embodiments, an adapter oligonucleotide comprises from 5′ to 3′, a third sequencing primer sequence (e.g., SBS3), a sequence complementary to a unique index sequence (e.g., i5′), and a second clustering primer sequence (e.g., P5). In some embodiments, an adapter comprises a sequence that is complementary to a primer. In further embodiments, an adapter comprises a sequence that is complementary to a P5 primer or a P5′ primer. In some embodiments, an adapter comprises a sequence complementary to a P7 primer or a P7′ primer. In some embodiments, an adapter comprises a sequence complementary to a B15 primer or a B15′ primer.

The terms “P5”, “P7”, “B15”, “P5” (P5 prime), “P7” (P7 prime), “B15” (B15 prime), “P15”, and “P17” may be used when referring to examples of oligonucleotide sequences of primers, e.g., clustering primers, and/or oligonucleotide sequences that are complementary to primers. The terms “P5” (P5 prime), “P7” (P7 prime), and “B15” (B15 prime) refer to the complement of P5, P7, and B15, respectively. It will be understood that any suitable primer can be used in the methods presented herein, and that the use of P5, P5′, P7, P7′, P15, P17, B15, and B15′ are exemplary embodiments only. Uses of primers such as P5, P5′, P7, P7′, P15, P17, B15, and B15′ or their complements on flow cells are known in the art, as exemplified by the disclosures of WO 2019/222264, WO 2007/010251, WO 2006/064199, WO 2005/065814, WO 2015/106941, WO 1998/044151, and WO 2000/018957, each of which is incorporated herein by reference in its entirety. For example, any suitable forward amplification primer, whether immobilized or in solution, can be useful in the methods presented herein for hybridization to a complementary sequence and amplification of a sequence. Similarly, any suitable reverse amplification primer, whether immobilized or in solution, can be useful in the methods presented herein for hybridization to a complementary sequence and amplification of a sequence. One of skill in the art will understand how to design and use primer sequences that are suitable for capture and/or amplification of nucleic acids as presented herein. In some embodiments, a “first clustering primer” as described herein is a P5 primer. In some embodiments, a “first clustering primer” as described herein is a P7 primer. In some embodiments, a “first clustering primer” as described herein is a P5′ primer. In some embodiments, a “first clustering primer” as described herein is a P7′ primer. In some embodiments, a “second clustering primer” as described herein is a P5 primer. In some embodiments, a “second clustering primer” as described herein is a P7 primer. In some embodiments, a “second clustering primer” as described herein is a P5′ primer. In some embodiments, a “second clustering primer” as described herein is a P7′ primer. In some embodiments, P5 comprises or consists of the polynucleotide sequence 5′ AAT GAT ACG GOG ACC ACC GA 3′ (SEQ ID NO: 1), or a variant thereof. In some embodiments, P5 comprises or consists of the polynucleotide sequence 5′ AAT GAT ACG GOG ACC ACC GAG ATC TAC AC 3′ (SEQ ID NO: 2), or a variant thereof. In some embodiments, P7 comprises or consists of the polynucleotide sequence 5′ CAA GCA GAA GAC GGC ATA CG 3′ (SEQ ID NO. 3), or a variant thereof. In some embodiments, P7 comprises or consists of the polynucleotide sequence 5′ CAA GCA GAA GAC GGC ATA CGA GAT 3′ (SEQ ID NO. 4), or a variant thereof. In some embodiments, P5′ comprises or consists of the polynucleotide sequence 5′ TCG GTG GTC GCC GTA TCA TT 3′ (SEQ ID NO: 5), or a variant thereof. In some embodiments, P5′ comprises or consists of the polynucleotide sequence 5′ GTG TAG ATC TCG GTG GTC GCC GTA TCA TT 3′ (SEQ ID NO: 6), or a variant thereof. In some embodiments, P7′ comprises the polynucleotide sequence 5′ CGT ATG CCG TCT TCT GCT TG 3′ (SEQ ID NO. 7), or a variant thereof. In some embodiments, P7′ comprises or consists of the polynucleotide sequence 5′ ATC TCG TAT GCC GTC TTC TGC TTG 3′ (SEQ ID NO. 8), or a variant thereof. In some embodiments, B15 comprises or consists of the polynucleotide sequence 5′ GTCTCGTGGGCTCGG 3′ (SEQ ID NO: 9), or a variant thereof. In some embodiments, B15′ comprises or consists of the polynucleotide sequence 5′ CCGAGCCCACGAGAC 3′ (SEQ ID NO: 10), or a variant thereof. In some embodiments, P15 comprises or consists of the polynucleotide sequence 5′ TTTTTTAATG ATACGGCGAC CACCGAGANC TACAC 3′ (SEQ ID NO: 11), or a variant thereof. In some embodiments, P17 comprises or consists of the polynucleotide sequence 5′ TTTTTTNNNC AAGCAGAAGA CGGCATACGA GAT 3′ (SEQ ID NO: 12), or a variant thereof. The term “variant” as used herein with reference to any of the sequences recited herein refers to a variant nucleic acid that is substantially identical, i.e., has only some nucleotide sequence variations, for example to the non-variant sequence. In some embodiments, a variant has at least 50%, 55%, 60%, 65%, 70%, 75%, 80%, 85%, 86%, 87%, 88%, 89%, 90%, 91%, 92%, 93%, 94%, 95%, 96%, 97%, 98%, or at least 99% overall nucleotide sequence identity to the non-variant nucleic acid sequence. It will be understood that reference to P5 and P7 herein could refer to different primer sequences. Any suitable primer sequence combinations are encompassed by the present disclosure.

As used herein, the term “plurality” is intended to mean a population of two or more different members. Pluralities can range in size from small, medium, large, to very large. The size of small plurality can range, for example, from a few members to tens of members. Medium sized pluralities can range, for example, from tens of members to about 100 members or hundreds of members. Large pluralities can range, for example, from about hundreds of members to about 1000 members, to thousands of members and up to tens of thousands of members. Very large pluralities can range, for example, from tens of thousands of members to about hundreds of thousands, a million, millions, tens of millions and up to or greater than hundreds of millions of members. Therefore, a plurality can range in size from two to well over one hundred million members as well as all sizes, as measured by the number of members, in between and greater than the above exemplary ranges. An exemplary number of features within a microarray includes a plurality of about 500,000 or more discrete features within 1.28 cm². Exemplary nucleic acid pluralities include, for example, populations of about 1×10⁵, 5×10⁵ and 1×10⁶ or more different nucleic acid species. Accordingly, the definition of the term is intended to include all integer values greater than two. An upper limit of a plurality can be set, for example, by the theoretical diversity of nucleotide sequences in a nucleic acid sample.

As used herein, the term “nucleic acid” is intended to be consistent with its use in the art and includes naturally occurring nucleic acids or functional analogs thereof. Particularly useful functional analogs are capable of hybridizing to a nucleic acid in a sequence specific fashion or capable of being used as a template for replication of a particular nucleotide sequence. Naturally occurring nucleic acids generally have a backbone containing phosphodiester bonds. An analog structure can have an alternate backbone linkage including any of a variety of those known in the art. Naturally occurring nucleic acids generally have a deoxyribose sugar (e.g., found in deoxyribonucleic acid (DNA)) or a ribose sugar (e.g., found in ribonucleic acid (RNA)). A nucleic acid can contain any of a variety of analogs of these sugar moieties that are known in the art. A nucleic acid can include native or non-native bases. In this regard, a native deoxyribonucleic acid can have one or more bases selected from the group consisting of adenine, thymine, cytosine or guanine and a ribonucleic acid can have one or more bases selected from the group consisting of uracil, adenine, cytosine or guanine. Useful non-native bases that can be included in a nucleic acid are known in the art. The term “target,” when used in reference to a nucleic acid, is intended as a semantic identifier for the nucleic acid in the context of a method or composition set forth herein and does not necessarily limit the structure or function of the nucleic acid beyond what is otherwise explicitly indicated. Particular forms of nucleic acids may include all types of nucleic acids found in an organism as well as synthetic nucleic acids such as polynucleotides produced by chemical synthesis.

Particular examples of nucleic acids that are applicable for analysis through incorporation into microarrays produced by methods as provided herein include genomic DNA (gDNA), expressed sequence tags (ESTs), DNA copied messenger RNA (cDNA), RNA copied messenger RNA (cRNA), mitochondrial DNA or genomic RNA, messenger RNA (mRNA), ribosomal RNA (rRNA) and/or other populations of RNA. Additional RNA contemplated include microRNA, transfer RNA, non-coding RNA (ncRNA), small nucleolar RNA (snoRNA), and/or small nuclear RNA (snRNA), Fragments and/or portions of these exemplary nucleic acids also are included within the meaning of the term as it is used herein.

As used herein, the term “double-stranded,” when used in reference to a nucleic acid molecule, means that substantially all of the nucleotides in the nucleic acid molecule are hydrogen bonded to a complementary nucleotide. A partially double stranded nucleic acid can have at least 10%, 25%, 50%, 60%, 70%, 80%, 90% or 95% of its nucleotides hydrogen bonded to a complementary nucleotide.

As used herein, the term “single-stranded,” when used in reference to a nucleic acid molecule, means that essentially none of the nucleotides in the nucleic acid molecule are hydrogen bonded to a complementary nucleotide.

As used herein, the term “capture primers” or “capture probe” is intended to mean an oligonucleotide having a nucleotide sequence that is capable of specifically annealing to a single stranded polynucleotide sequence to be analyzed or subjected to a nucleic acid interrogation under conditions encountered in a primer annealing step of, for example, an amplification or sequencing reaction. The terms “nucleic acid,” “polynucleotide” and “oligonucleotide” are used interchangeably herein. The different terms are not intended to denote any particular difference in size, sequence, or other property unless specifically indicated otherwise. For clarity of description the terms can be used to distinguish one species of nucleic acid from another when describing a particular method or composition that includes several nucleic acid species.

As used herein, the term “gene-specific” or “target specific” when used in reference to a capture probe or other nucleic acid is intended to mean a capture probe or other nucleic acid that includes a nucleotide sequence specific to a targeted nucleic acid, e.g., a nucleic acid from a tissue sample, namely a sequence of nucleotides capable of selectively annealing to an identifying region of a targeted nucleic acid. Gene-specific capture probes can have a single species of oligonucleotide, or can include two or more species with different sequences. Thus, the gene-specific capture probes can be two or more sequences, including 3, 4, 5, 6, 7, 8, 9 or 10 or more different sequences. The gene-specific capture probes can comprise a gene-specific capture primer sequence and a universal capture probe sequence. Other sequences such as sequencing primer sequences and the like also can be included in a gene-specific capture primer.

As used herein “unique molecular index”, “unique molecular identifier” or “UMI”, when used in reference to a capture probe or other nucleic acid is intended to refer to a portion of a probe useful as a molecular barcode to uniquely tag each molecule in a sample library. A UMI may be denoted as “NNNN . . . ” in a string of nucleic acids to designate that portion of the oligonucleotide as the UMI. A UMI may be from 6 to 20 nucleotides or more in length. In some aspects, the UMI comprises a spatial barcode.

In comparison, the term “universal” when used in reference to a capture probe or other nucleic acid is intended to mean a capture probe or nucleic acid having a common nucleotide sequence among a plurality of capture probes. A common sequence can be, for example, a sequence complementary to the same adapter sequence. Universal capture probes are applicable for interrogating a plurality of different polynucleotides without necessarily distinguishing the different species whereas gene-specific capture primers are applicable for distinguishing the different species.

In various embodiments, the capture elements (e.g., capture primers or capture probes or other nucleic acid sequences) can be spaced to A) spatially resolve nucleic acids within the geometry of a single cell, i.e., multiple capture sites per cell; B) spatially resolve nucleic acids at about the single cell level, i.e., about 1 capture site per cell. Additionally, capture elements may be spaced as in A or B above, and be: I) spaced to sample nucleic acids from a sample at regular intervals, e.g., spaced in a grid or pattern such that about every other or every 5th or every 10th cell is sampled, or about every other or every 5th or every 10th group of 2, 3, 4, 5, 6, 7, 8, 9, 10 or more cells is sampled; II) spaced to capture samples from substantially all available cells in one or more regions of a sample, or III) spaced to capture samples from substantially all available cells in the sample.

As used herein, the term “amplicon,” when used in reference to a nucleic acid, means the product of copying the nucleic acid, wherein the product has a nucleotide sequence that is the same as or complementary to at least a portion of the nucleotide sequence of the nucleic acid. An amplicon can be produced by any of a variety of amplification methods that use the nucleic acid, or an amplicon thereof, as a template including, for example, polymerase extension, polymerase chain reaction (PCR), rolling circle amplification (RCA), ligation extension, or ligation chain reaction. An amplicon can be a nucleic acid molecule having a single copy of a particular nucleotide sequence (e.g., a PCR product) or multiple copies of the nucleotide sequence (e.g., a concatameric product of RCA). A first amplicon of a target nucleic acid can be a complementary copy. Subsequent amplicons are copies that are created, after generation of the first amplicon, from the target nucleic acid or from the first amplicon. A subsequent amplicon can have a sequence that is substantially complementary to the target nucleic acid or substantially identical to the target nucleic acid.

The number of template copies or amplicons that can be produced can be modulated by appropriate modification of the amplification reaction including, for example, varying the number of amplification cycles run, using polymerases of varying processivity in the amplification reaction and/or varying the length of time that the amplification reaction is run, as well as modification of other conditions known in the art to influence amplification yield. The number of copies of a nucleic acid template can be at least 1, 10, 100, 200, 500, 1000, 2000, 3000, 4000, 5000, 6000, 7000, 8000, 9000 and 10,000 copies, and can be varied depending on the particular application.

As used herein, the term “complementary” when used in reference to a polynucleotide is intended to mean a polynucleotide that includes a nucleotide sequence capable of selectively annealing to an identifying region of a target polynucleotide under certain conditions. As used herein, the term “substantially complementary” and grammatical equivalents is intended to mean a polynucleotide that includes a nucleotide sequence capable of specifically annealing to an identifying region of a target polynucleotide under certain conditions. Annealing refers to the nucleotide base-pairing interaction of one nucleic acid with another nucleic acid that results in the formation of a duplex, triplex, or other higher-ordered structure. The primary interaction is typically nucleotide base specific, e.g., A:T, A:U, and G:C, by Watson-Crick and Hoogsteen-type hydrogen bonding. In certain embodiments, base-stacking and hydrophobic interactions can also contribute to duplex stability. Conditions under which a polynucleotide anneals to complementary or substantially complementary regions of target nucleic acids are well known in the art, e.g., as described in Nucleic Acid Hybridization, A Practical Approach, Hames and Higgins, eds., IRL Press, Washington, D.C. (1985) and Wetmur and Davidson, Mol. Biol. 31:349 (1968). Annealing conditions will depend upon the particular application and can be routinely determined by persons skilled in the art, without undue experimentation.

As used herein, the term “hybridization” refers to the process in which two single-stranded polynucleotides bind non-covalently to form a stable double-stranded polynucleotide. A resulting double-stranded polynucleotide is a “hybrid” or “duplex.” Hybridization conditions will typically include salt concentrations of less than about 1 M, more usually less than about 500 mM and may be less than about 200 mM. A hybridization buffer includes a buffered salt solution such as 5% SSPE, or other such buffers known in the art. Hybridization temperatures can be as low as 5° C., but are typically greater than 22° C., and more typically greater than about 30° C., and typically in excess of 37° C. Hybridizations are usually performed under stringent conditions, i.e., conditions under which a probe will hybridize to its target subsequence but will not hybridize to the other, uncomplimentary sequences. Stringent conditions are sequence-dependent and are different in different circumstances, and may be determined routinely by those skilled in the art.

As used herein, the term “dNTP” refers to deoxynucleoside triphosphates. NTP refers to ribonucleotide triphosphates. The purine bases (Pu) include adenine (A), guanine (G) and derivatives and analogs thereof. The pyrimidine bases (Py) include cytosine (C), thymine (T), uracil (U) and derivatives and analogs thereof. Examples of such derivatives or analogs, by way of illustration and not limitation, are those which are modified with a reporter group, biotinylated, amine modified, radiolabeled, alkylated, and the like and also include phosphorothioate, phosphite, ring atom modified derivatives, and the like. The reporter group can be a fluorescent group such as fluorescein, a chemiluminescent group such as luminol, a terbium chelator such as N-(hydroxyethyl)ethylenediaminetriacetic acid that is capable of detection by delayed fluorescence, and the like.

As used herein, the terms “ligation,” “ligating,” and grammatical equivalents thereof are intended to mean to form a covalent bond or linkage between the termini of two or more nucleic acids, e.g., oligonucleotides and/or polynucleotides, typically in a template-driven reaction. The nature of the bond or linkage may vary widely, and the ligation may be carried out enzymatically or chemically. As used herein, ligations are usually carried out enzymatically to form a phosphodiester linkage between a 5′ carbon terminal nucleotide of one oligonucleotide with a 3′ carbon of another nucleotide. Template driven ligation reactions are described in the following references: U.S. Pat. Nos. 4,883,750; 5,476,930; 5,593,826; and 5,871,921, incorporated herein by reference in their entireties. The term “ligation” also encompasses non-enzymatic formation of phosphodiester bonds, as well as the formation of non-phosphodiester covalent bonds between the ends of oligonucleotides, such as phosphorothioate bonds, disulfide bonds, and the like.

As used herein, the term “each,” when used in reference to a collection of items, is intended to identify an individual item in the collection but does not necessarily refer to every item in the collection unless the context clearly dictates otherwise.

As used herein, the term “extend,” when used in reference to a nucleic acid, is intended to mean addition of at least one nucleotide or oligonucleotide to the nucleic acid. In particular embodiments one or more nucleotides can be added to the 3′ end of a nucleic acid, for example, via polymerase catalysis (e.g., DNA polymerase, RNA polymerase or reverse transcriptase). Chemical or enzymatic methods can be used to add one or more nucleotide to the 3′ or 5′ end of a nucleic acid. One or more oligonucleotides can be added to the 3′ or 5′ end of a nucleic acid, for example, via chemical or enzymatic (e.g., ligase catalysis) methods. A nucleic acid can be extended in a template directed manner, whereby the product of extension is complementary to a template nucleic acid that is hybridized to the nucleic acid that is extended.

Provided herein are arrays for and methods of spatial detection and analysis (e.g., mutational analysis or single nucleotide variation (SNV) detection as well as indel detection) of nucleic acid in a tissue sample. The arrays described herein can comprise a substrate on which a plurality of capture probes are immobilized such that each capture probe occupies a distinct position on the array. Some or all of the plurality of capture probes can comprise a unique positional tag (i.e., a spatial address or indexing sequence). A spatial address can describe the position of the capture probe on the array. The position of the capture probe on the array can be correlated with a position in the tissue sample.

As used herein, the term “poly T” or “poly A,” when used in reference to a nucleic acid sequence, is intended to mean a series of two or more thiamine (T) or adenine (A) bases, respectively. A poly T or poly A can include at least about 2, 5, 8, 10, 12, 15, 18, 20 or more of the T or A bases, respectively. Alternatively or additionally, a poly T or poly A can include at most about, 30, 20, 18, 15, 12, 10, 8, 5 or 2 of the T or A bases, respectively.

As used herein, the term “poly T”, “poly A,” or “poly U” when used in reference to a nucleic acid sequence (e.g., a capture nucleotide sequence), is intended to mean a series of two or more thiamine (T), adenine (A) or uridine (U) bases, respectively. A poly T or poly A or poly U can include at least about 2, 5, 8, 10, 12, 15, 18, 20, 22, 25, 28, 30, 32, 35, 38, 40, or more of the T or A bases, respectively. Alternatively, or additionally, a poly T or poly A or poly U can include at most about 40, 38, 35, 32, 30, 28, 25, 22, 20, 18, 15, 12, 10, 8, 5, or 2 of the T or A bases, respectively. In some embodiments, the disclosure contemplates use of a “TVN” sequence, wherein “T” is a capture nucleotide sequence, “V” is adenine (A), cytosine (C), or guanine (G), and “N” is adenine (A), cytosine (C), guanine (G), or thymine (T). The TVN sequence is used, in some embodiments, to bias reverse transcription to the base of the poly A tail on a mRNA molecule.

As used herein, the term “tagmentation,” “tagment,” or “tagmenting” refers to transforming a nucleic acid, e.g., a DNA, into adaptor-modified templates in solution ready for cluster formation and sequencing by the use of transposase mediated fragmentation and tagging. This process often involves the modification of the nucleic acid by a transposome complex comprising transposase enzyme complexed with adaptors comprising transposon end sequence. Tagmentation results in the simultaneous fragmentation of the nucleic acid and ligation of the adaptors to the 5′ ends of both strands of duplex fragments. Following a purification step to remove the transposase enzyme, additional sequences are added to the ends of the adapted fragments by PCR.

A “transposase” refers to an enzyme that is capable of forming a functional complex with a transposon end-containing composition (e.g., transposons, transposon ends, transposon end compositions) and catalyzing insertion or transposition of the transposon end-containing composition into the double-stranded target nucleic acid with which it is incubated, for example, in an in vitro transposition reaction. A transposase as presented herein can also include integrases from retrotransposons and retroviruses. Transposases, transposomes and transposome complexes are generally known to those of skill in the art, as exemplified by the disclosure of US Pat. Publ. No. 2010/0120098, the content of which is incorporated herein by reference in its entirety. Although many embodiments described herein refer to Tn5 transposase and/or hyperactive Tn5 transposase, it will be appreciated that any transposition system that is capable of inserting a transposon end with sufficient efficiency to 5′-tag and fragment a target nucleic acid for its intended purpose can be used in the present invention. In particular embodiments, a preferred transposition system is capable of inserting the transposon end in a random or in an almost random manner to 5′-tag and fragment the target nucleic acid.

As used herein, the term “transposition reaction” refers to a reaction wherein one or more transposons are inserted into target nucleic acids, e.g., at random sites or almost random sites. Essential components in a transposition reaction are a transposase and DNA oligonucleotides that exhibit the nucleotide sequences of a transposon, including the transferred transposon sequence and its complement (the non-transferred transposon end sequence) as well as other components needed to form a functional transposition or transposome complex. The DNA oligonucleotides can further comprise additional sequences (e.g., adaptor or primer sequences) as needed or desired. In some embodiments, the method provided herein is exemplified by employing a transposition complex formed by a hyperactive Tn5 transposase and a Tn5-type transposon end (Goryshin and Reznikoff, 1998, J. Biol. Chem., 273:7367) or by a MuA transposase and a Mu transposon end comprising Rland R2 end sequences (Mizuuchi, 1983, Cell, 35:785; Savilahti et al., 1995, EMBO J., 14:4893). However, any transposition system that is capable of inserting a transposon end in a random or in an almost random manner with sufficient efficiency to 5′-tag and fragment a target DNA for its intended purpose can be used in the present invention. Examples of transposition systems known in the art which can be used for the present methods include but are not limited to Staphylococcus aureus Tn552 (Colegio et al., 2001, J Bacterid., 183:2384-8; Kirby et al., 2002, Mol Microbiol, 43:173-86), Tyl (Devine and Boeke, 1994, NucleicAcids Res., 22:3765-72 and International Patent Application No. WO 95/23875), TransposonTn7 (Craig, 1996, Science. 271:1512; Craig, 1996, Review in: Curr Top MicrobiolImmunol, 204:27-48), TnIO and ISIO (Kleckner et al., 1996, Curr Top Microbiol Immunol, 204:49-82), Mariner transposase (Lampe et al., 1996, EMBO J., 15:5470-9), Tci (Plasterk, 1996, Curr Top Microbiol Immunol, 204:125-43), P Element (Gloor, 2004, Methods Mol Biol, 260:97-114), TnJ (Ichikawa and Ohtsubo, 1990, J Biol Chem. 265:18829-32), bacterial insertion sequences (Ohtsubo and Sekine, 1996, Curr. Top. Microbiol. Immunol. 204:1-26), retroviruses (Brown et al., 1989, Proc Natl Acad Sci USA, 86:2525-9), and retrotransposon of yeast (Boeke and Corces, 1989, Annu Rev Microbiol. 43:403-34). The method for inserting a transposon end into a target sequence can be carried out in vitro using any suitable transposon system for which a suitable in vitro transposition system is available or that can be developed based on knowledge in the art. In general, a suitable in vitro transposition system for use in the methods provided herein requires, at a minimum, a transposase enzyme of sufficient purity, sufficient concentration, and sufficient in vitro transposition activity and a transposon end with which the transposase forms a functional complex with the respective transposase that is capable of catalyzing the transposition reaction. Suitable transposase transposon end sequences that can be used in the invention include but are not limited to wild-type, derivative or mutant transposon end sequences that form a complex with a transposase chosen from among a wild-type, derivative or mutant form of the transposase. As used herein, the term “transposome complex” refers to a transposase enzyme non-covalently bound to a double stranded nucleic acid. For example, the complex can be a transposase enzyme pre-incubated with double-stranded transposon DNA under conditions that support non-covalent complex formation. Double-stranded transposon DNA can include, without limitation, Tn5 DNA, a portion of Tn5 DNA, a transposon end composition, a mixture of transposon end compositions or other double-stranded DNAs capable of interacting with a transposase such as the hyperactive Tn5 transposase.

As used herein, the term “random” can be used to refer to the spatial arrangement or composition of locations on a surface. For example, there are at least two types of order for an array described herein, the first relating to the spacing and relative location of features (also called “sites”) and the second relating to identity or predetermined knowledge of the particular species of molecule that is present at a particular feature. Accordingly, features of an array can be randomly spaced such that nearest neighbor features have variable spacing between each other. Alternatively, the spacing between features can be ordered, for example, forming a regular pattern such as a rectilinear grid or hexagonal grid. In another respect, features of an array can be random with respect to the identity or predetermined knowledge of the gene of interest (e.g. nucleic acid of a particular sequence) that occupies each feature independent of whether spacing produces a random pattern or ordered pattern. An array set forth herein can be ordered in one respect and random in another. For example, in some embodiments set forth herein a surface is contacted with a population of nucleic acids under conditions where the nucleic acids attach at sites that are ordered with respect to their relative locations but ‘randomly located’ with respect to knowledge of the sequence for the nucleic acid species present at any particular site. Reference to “randomly distributing” nucleic acids at locations on a surface is intended to refer to the absence of knowledge or absence of predetermination regarding which nucleic acid will be captured at which location (regardless of whether the locations are arranged in an ordered pattern or not).

As used herein, a “biological sample” may include one or more biological or chemical substances, such as nucleic acids, oligonucleotides, proteins, cells, tissues, organisms, and/or biologically active chemical compound(s), such as analogs or mimetics of the aforementioned species. As used herein, the term “tissue” is intended to mean an aggregation of cells, and, optionally, intercellular matter. Typically, the cells in a tissue are not free floating in solution and instead are attached to each other to form a multicellular structure. Exemplary tissue types include muscle, nerve, epidermal and connective tissues. In some instances, the biological sample may include whole blood, lymphatic fluid, serum, plasma, sweat, tear, saliva, sputum, cerebrospinal fluid, amniotic fluid, seminal fluid, vaginal excretion, serous fluid, synovial fluid, pericardial fluid, peritoneal fluid, pleural fluid, transudates, exudates, cystic fluid, bile, urine, gastric fluid, intestinal fluid, fecal samples, liquids containing single or multiple cells, liquids containing organelles, fluidized tissues, fluidized organisms, viruses including viral pathogens, liquids containing multi-celled organisms, biological swabs and biological washes. In further examples, the sample can be derived from an organ, including for example, an organ of the musculoskeletal system such as muscle, bone, tendon or ligament; an organ of the digestive system such as salivary gland, pharynx, esophagus, stomach, small intestine, large intestine, liver, gallbladder or pancreas; an organ of the respiratory system such as larynx, trachea, bronchi, lungs or diaphragm; an organ of the urinary system such as kidney, ureter, bladder or urethra; a reproductive organ such as ovary, fallopian tube, uterus, vagina, placenta, testicle, epididymis, vas deferens, seminal vesicle, prostate, penis or scrotum; an organ of the endocrine system such as pituitary gland, pineal gland, thyroid gland, parathyroid gland, or adrenal gland; an organ of the circulatory system such as heart, artery, vein or capillary; an organ of the lymphatic system such as lymphatic vessel, lymph node, bone marrow, thymus or spleen; an organ of the central nervous system such as brain, brainstem, cerebellum, spinal cord, cranial nerve, or spinal nerve; a sensory organ such as eye, ear, nose, or tongue; or an organ of the integument such as skin, subcutaneous tissue or mammary gland. In various embodiments, the tissue can be derived from a multicellular organism. In some embodiments, a tissue section can be contacted with a surface, for example, by laying the tissue on the surface. The tissue can be freshly excised from an organism, or it may have been previously preserved for example by freezing (e.g., fresh frozen tissue), embedding in a material such as paraffin (e.g., formalin fixed paraffin embedded (FFPE) samples), formalin fixation, infiltration, dehydration or the like. Optionally, a tissue section can be attached to a surface, for example, using techniques and compositions described in, for example, U.S. Pat. No. 11,390,912, incorporated by reference herein in its entirety. In some embodiments, a tissue can be permeabilized and the cells of the tissue lysed when the tissue is in contact with a surface. Any of a variety of treatments can be used such as those set forth above in regard to lysing cells. Target proteins and/or nucleic acids that are released from a tissue that is permeabilized can be captured by capture oligonucleotides on the surface. Thus, in various embodiments, the biological sample is a tissue sample. The thickness of a tissue sample or other biological sample that is contacted with a surface in a method set forth herein can be any suitable thickness desired. In representative embodiments, the thickness will be at least 0.1 μm, 0.25 μm, 0.5 μm, 0.75 μm, 1 μm, 5 μm, 10 μm, 50 μm, 100 μm or thicker. Alternatively or additionally, the thickness of a biological sample that is contacted with a surface will be no more than 100 μm, 50 μm, 10 μm, 5 μm, 1 μm, 0.5 μm, 0.25 μm, 0.1 μm or thinner.

As used herein, the term “tissue sample” refers to a piece of tissue that has been obtained from a subject, fixed, sectioned, and mounted on a planar surface, e.g., a microscope slide. The tissue sample can be a formalin-fixed paraffin-embedded (FFPE) tissue sample or a fresh tissue sample or a frozen tissue sample, etc. The methods disclosed herein may be performed before or after staining the tissue sample. For example, following hematoxylin and eosin staining, a tissue sample may be spatially analyzed in accordance with the methods as provided herein. A method may include analyzing the histology of the sample (e.g., using hematoxylin and esoins staining) and then spatially analyzing the tissue.

As used herein, the term “formalin-fixed paraffin embedded (FFPE) tissue section” refers to a piece of tissue, e.g., a biopsy that has been obtained from a subject, fixed in formaldehyde (e.g., 3%-5% formaldehyde in phosphate buffered saline) or Bouin solution, embedded in wax, cut into thin sections, and then mounted on a planar surface, e.g., a microscope slide.

As used herein, the term “subject” encompasses mammals and non-mammals. Examples of mammals include, but are not limited to, any member of the mammalian class: humans, non-human primates such as chimpanzees, and other apes and monkey species, cattle, horses, sheep, goats, swine, rabbits, dogs, cats, rodents, rats, mice, guinea pigs, and the like. Examples of non-mammals include, but are not limited to, birds, fish, and the like. The term does not denote a particular age or gender.

In some embodiments, nucleic acids in a tissue sample are transferred to and captured onto an array. For example, a tissue section is placed in contact with an array and nucleic acid is captured onto the array and tagged with a spatial address. The spatially-tagged DNA molecules are released from the array and analyzed, for example, by high throughput next generation sequencing (NGS), such as sequencing-by-synthesis (SBS). In some embodiments, a nucleic acid in a tissue section (e.g., a formalin-fixed paraffin-embedded (FFPE) tissue section) is transferred to an array and captured onto the array by hybridization to a capture probe. In some embodiments, a capture probe can be a universal capture probe hybridizing, e.g., to an adaptor region in a nucleic acid sequencing library, or to the poly-A tail of an mRNA. Alternatively, the spatially-tagged RNA or DNA molecules are released from the array and analyzed, for example, by high throughput next generation sequencing (NGS), such as sequencing-by-synthesis (SBS). In some embodiments, a nucleic acid in a tissue section (e.g., a formalin-fixed paraffin-embedded (FFPE) tissue section) is transferred to an array and captured onto the array by hybridization to a capture probe. In some embodiments, the capture probe can be a gene-specific capture probe hybridizing, e.g., to a specifically targeted mRNA or cDNA in a sample, such as a TruSeq™ Custom Amplicon (TSCA) oligonucleotide probe (Illumina, Inc.). A capture probe can be a plurality of capture probes, e.g., a plurality of the same or of different capture probes.

In some embodiments, a combinatorial indexing (addressing) system is used to provide spatial information for analysis of nucleic acids in a tissue sample. The combinatorial indexing system can involve the use of two or more spatial address sequences (e.g., two, three, four, five or more spatial address sequences).

In some embodiments, two spatial address sequences are incorporated into a nucleic acid during preparation of a sequencing library. A first spatial address can be used to define a certain position (i.e., capture site) in the X dimension on a capture array and a second spatial address sequence can be used define a position (i.e., a capture site) in the Y dimension on the capture array. During library sequencing, both X and Y spatial address sequences can be determined and the sequence information can be analyzed to define the specific position on the capture array.

In some embodiments, three spatial address sequences are incorporated into a nucleic acid during preparation of a sequencing library. A first spatial address can be used to define a certain position (i.e., capture site) in the X dimension on a capture array, a second spatial address sequence can be used define a position (i.e., a capture site) in the Y dimension on the capture array, and a third spatial address sequence can be used to define a position of a two-dimensional sample section (e.g., the position of a slice of a tissue sample) in a sample (e.g., a tissue biopsy) to provide positional spatial information in the third dimension (Z dimension) of a sample. During library sequencing, X, Y, and Z spatial address sequences can be determined and the sequence information can be analyzed to define the specific position on the capture array.

In some embodiments, a temporal address sequence (T) is optionally incorporated into a nucleic acid during preparation of a sequencing library. In some embodiments, the temporal address sequence can be combined with two or three spatial address sequences. The temporal address sequence can, for example, be used in the context of a time-course experiment for determining time-dependent changes in gene-expression in a tissue sample. Time-dependent changes in gene-expression can occur in a tissue sample, for example, in response to a chemical, biological or physical stimulus (e.g., a toxin, a drug, or heat). Nucleic acid samples obtained at different timepoints from comparable tissue samples (e.g., proximal slices of a tissue sample) can be pooled and sequenced in bulk. An optional first spatial address can be used to define a certain position (i.e., capture site) in the X dimension on a capture array, a second optional spatial address sequence can be used to define a position (i.e., a capture site) in the Y dimension on the capture array, and a third optional spatial address sequence can be used to define a position of a two-dimensional sample section (e.g., the position of a slice of a tissue sample) in a sample (e.g., a tissue biopsy) to provide positional spatial information in the third dimension (Z dimension) of the sample. During library sequencing, T, X, Y, and Z address sequences are determined and the sequence information is analyzed to define the specific X, Y (and optionally Z) position on the capture array for each timepoint (T).

The address sequences X, Y, and, optionally, Z and/or T, can be consecutive nucleic acid sequences or the address sequences can be separated by one or more nucleic acids (e.g., 2 or more, 3 or more, 10 or more, 30 or more, 100 or more, 300 or more, or 1,000 or more). In some embodiments, the X, Y, and optionally Z and/or T address sequences can each individually and independently be combinatorial nucleic acid sequences.

In some embodiments, the length of the address sequences (e.g., X, Y, Z, or T) can each individually and independently be 100 nucleic acids or less, 90 nucleic acids or less, 80 nucleic acids or less, 70 nucleic acids or less, 60 nucleic acids or less, 50 nucleic acids or less, 40 nucleic acids or less, 30 nucleic acids or less, 20 nucleic acids or less, 15 nucleic acids or less, 10 nucleic acids or less, 8 nucleic acids or less, 6 nucleic acids or less, or 4 nucleic acids or less. The length of two or more address sequences in a nucleic acid can be the same or different. For example, if the length of address sequence X is 10 nucleic acids, the length of address sequence Y can be, e.g., 8 nucleic acids, 10 nucleic acids, or 12 nucleic acids.

Address sequences, e.g., spatial address sequences such as X or Y, can be either partially or fully degenerate sequences.

In some embodiments, spatially addressed capture probes on an array can be released from the array onto a tissue section for generation of a spatially addressed sequencing library. In some embodiments, a capture probe comprises a random primer sequence for in situ synthesis of spatially-tagged cDNA from RNA in the tissue section. In some embodiments, a capture probe is a TruSeq™ Custom Amplicon (TSCA) oligonucleotide probe (Illumina, Inc.) for capturing and spatially tagging genomic DNA in the tissue section. The spatially-tagged nucleic acid molecules (e.g., cDNA or genomic DNA) are recovered from the tissue section and processed in single tube reactions to generate a spatially-tagged amplicon library.

In another embodiment, the disclosure provides for substrates, e.g., flowcell, nanoparticles or beads which comprise the spatially addressable probes disclosed herein. In a particular embodiment, beads comprise the spatially addressable probes disclosed herein. In a further embodiment, the substrate comprises streptavidin on the surface of the bead. In yet a further embodiment, the beads comprise a plurality of oligos bound to the bead via a linkage or a reversible linkage. Examples of reversible linkages include biotin molecule(s), such as ddBio molecules. The oligos bound the substrate typically comprise an adaptor sequence, such as P5 sequence or a P7 sequence. As used herein a P5 sequence comprises a sequence comprising AAT GAT ACG GCG ACC ACC GA (SEQ ID NO: 1) or AAT GAT ACG GCG ACC ACC GAG ATC TAC AC (SEQ ID NO: 2) and a P7 sequence comprises a sequence CAA GCA GAA GAC GGC ATA CG (SEQ ID NO: 3) or CAA GCA GAA GAC GGC ATA CGA GAT (SEQ ID NO: 4). In some embodiments, the P5 or P7 sequence can further include a spacer polynucleotide, which may be from 1 to 20, such as 1 to 15, or 1 to 10, nucleotides, such as 2, 3, 4, 5, 6, 7, 8, 9, or 10 nucleotides in length. In some embodiments, the spacer includes 10 nucleotides. In some embodiments, the spacer includes 10 nucleotides. In some embodiments, the spacer is a polyT spacer, such as a 10T spacer. Spacer nucleotides may be included at the 5′ ends of polynucleotides, which may be attached to a suitable support via a linkage with the 5′ end of the oligo. Attachment can be achieved through a sulfur-containing nucleophile, such as phosphorothioate, present at the 5′ end of the polynucleotide. In some embodiments, the oligos will include a polyT spacer and a 5′ phosphorothioate group. Thus, in some embodiments, the P5 sequence comprises 5′ phosphorothioate-TTTTTTTTTTAATGATACGGCGACCACCGA-3′ (SEQ ID NO: 17), and in some embodiments, the P7 sequence comprises 5′ phosphorothioate-TTTTTTTTTTCAAGCAGAAGACGGCATACGA-3′ (SEQ ID NO: 18). In certain embodiments, the oligos attached to the substrate comprise an address sequence that allows for determining the x, y position of the oligo when decoded. In further embodiments, the address sequence is 5, 10, 15, 20, 25, 30, 35, 40, 45, 50, 55, 60, 65, 70, 75, 80, 85, 90, 95, or 100 nucleotides in length, or a range that includes or is between any two of the foregoing nucleotides in length. In another embodiment, the oligos comprise a transposome hybridization region (Tsm hyb). In yet additional embodiments, the oligos comprise sequencing primer(s) site sequence(s). Examples of sequencing primer site sequences include sequences that are complementary to R1 and R2 sequencing primers from Illumina™. In further embodiments, the oligos may further comprise one or more linker sequences. In yet further embodiment, the oligos may further comprise one or more index sequences. In certain embodiments, the oligos may comprise one or more unique molecular identifier (UMI) sequences. Unique molecular identifiers (UMIs) are a type of molecular barcoding that provides error correction and increased accuracy during sequencing. These molecular barcodes are short sequences used to uniquely tag each molecule in a sample library. UMIs are used for a wide range of sequencing applications, many around PCR duplicates in DNA and cDNA. UMI deduplication is also useful for RNA-seq gene expression analysis and other quantitative sequencing methods. As noted previously, the oligos comprise moieties or sequences that can bind with specificity to polynucleotides from a biological sample (e.g., a tissue sample). As such, the oligos are spatially addressable probes for polynucleotides from a biological sample. The moieties or sequences that can bind with specificity to polynucleotides from a biological sample can be selected for a particular omic application. For example, the oligos can comprise an oligo d (T) sequence for transcriptomics or for assay (e.g., RNA-seq assays). Alternatively, the oligos can comprise sequences to bind with genomic DNA from a biological sample for genomic applications or for assays (e.g., ATAC-seq assays). As provided in the Examples presented herein, the substrate can comprise multiple types of oligos that have different moieties or sequences so that the spatially addressable probes can bind specifically to two or more different types of polynucleotides from a biological sample. The use of multi types of oligos is ideally suited for multiomic or multiple assay applications.

In some embodiments, magnetic nanoparticles can be used to capture nucleic acid (e.g., in situ synthesized cDNA) in a tissue sample for generation of a spatially addressed library.

In some embodiments, spatial detection and analysis of nucleic acid in a tissue sample can be performed on a droplet actuator.

Described herein are improved methods and compositions for spatial-omics applications that preserve spatial information related to the origin of RNA or DNA in the tissue. Examples of spatial omics applications include, but are not limited to, spatial genomic applications, spatial proteomic applications; spatial transcriptomic applications; spatial agrigenomic applications; spatial epigenomics s applications; spatial phenomic applications; spatial ligandomic applications; and spatial multiomic applications (e.g., transcriptomic and genomic applications).

### Isolation of Polynucleotides

In various embodiments, one or more samples that have been contacted with a solid support can be lysed to release target nucleic acids. Lysis can be carried out using methods known in the art such as those that employ one or more of chemical treatment, enzymatic treatment, electroporation, heat, hypotonic treatment, sonication or the like.

In some embodiments, a tissue sample will be treated to remove embedding material (e.g., to remove paraffin or formalin) from the sample prior to release, capture or modification of nucleic acids. This can be achieved by contacting the sample with an appropriate solvent (e.g., xylene and ethanol washes). Treatment can occur prior to contacting the tissue sample with a solid support set forth herein or the treatment can occur while the tissue sample is on the solid support. Exemplary methods for manipulating tissues for use with solid supports to which nucleic acids are attached are set forth in US Pat. App. Publ. No. 2014/0066318, which is incorporated herein by reference.

### Preparation of Polynucleotides

The present disclosure is based, in part, on the realization that the amount of RNA or DNA information isolatable from fresh or frozen tissue samples as well as FFPE tissue samples needs to be improved to provide information related to the genetic profile of the tissue sample. The present disclosure provides methods for improved capture of genetic information by increasing the amount and quality of RNA isolated from tissue samples that can be used in spatial transcriptomics analysis.

The total RNA can comprise ribosomal RNA (rRNA), messenger RNA (mRNA), transfer RNA (IRNA), microRNA (miRNA), non-coding RNA (ncRNA), small nucleolar RNA (snoRNA), and/or small nuclear RNA (snRNA). In various embodiments, the RNA is rRNA and/or mRNA.

In various embodiments, the RNA capture probe is selected from the group consisting of a poly-T sequence, a poly-U sequence, a randomer, a semi-random sequence, or a target-specific probe. In various embodiments, the target-specific probes comprise a plurality of different target-specific RNA capture probe sequences. In various embodiments, the RNA capture probe or surface capture probe is between 8 to 80 nucleotides. In certain embodiments, the RNA capture probe or surface probe is between 10 to 80 nucleotides, between 10 to 70 nucleotides, between 10 to 60 nucleotides, between 10 to 50 nucleotides, between 10 to 40 nucleotides, between 10 to 30 nucleotides, between 10 to 20 nucleotides, between 20 to 80 nucleotides, between 20 to 70 nucleotides, between 20 to 60 nucleotides, between 20 to 50 nucleotides, between 20 to 40 nucleotides, or is 8, 9, 10, 11, 12, 13, 14, 15, 16, 17, 18, 19, 20, 21, 22, 23, 24, 25, 26, 27, 28, 29, 30, 40, 50, 60, 70 or 80 nucleotides.

In various embodiments, a capture oligonucleotide comprises a clustering primer sequence and a capture nucleotide sequence that is configured to bind to target nucleic acids of a biological sample. In some embodiments, a capture oligonucleotide comprises a clustering primer sequence (e.g., a P7 sequence), a spatial barcode (SBC) sequence, a sequencing primer sequence (e.g., a sequencing by synthesis (SBS) sequence such as SBS12), a single molecule identifier (SMI) sequence, a quality control sequence, and a TVN sequence, wherein “T” is a capture nucleotide sequence, “V” is adenine (A), cytosine (C), or guanine (G), and “N” is adenine (A), cytosine (C), guanine (G), or thymine (T). In various embodiments, a capture oligonucleotide is between about 30 bases to about 100 bases in length, or between about 30 bases to about 90 bases, or between about 30 bases and 80 bases, or between about 30 bases and 70 bases, or between about 30 bases and 60 bases, or between about 30 bases and 55 bases, or between about 30 bases and 50 bases in length or between 20 bases to 80 bases, or between 10 bases to 80 bases. In further embodiments, a capture oligonucleotide of the disclosure is about 10 bases, 20 bases, 30 bases, 35 bases, 40 bases, 45 bases, 50 bases, 55 bases, 60 bases, 65 bases, 70 bases, 75 bases, 80 bases, 85 bases, 90 bases, 95 bases, or 100 bases in length. The capture nucleotide sequence capable of hybridizing or otherwise associating with an analyte (e.g., a target nucleic acid) is, for example and without limitation, a universal sequence (e.g., a poly T sequence, a random nucleotide sequence, or a semi-random nucleotide sequence), or a target-specific (e.g., a gene-specific) sequence. In various embodiments, a capture nucleotide sequence (e.g., a poly T nucleotide sequence or a random nucleotide sequence) is, is about, or is at least about 2, 5, 8, 10, 12, 15, 18, 20, 22, 25, 28, 30, 32, 35, 38, 40, 45, 50, or more bases in length. Alternatively or additionally, a capture nucleotide sequence can include less than or equal to about 50, 45, 40, 38, 35, 32, 30, 28, 25, 22, 20, 18, 15, 12, 10, 8, 5, or 2 bases. A capture oligonucleotide can comprise additional elements, including but not limited to a single molecule identifier (SMI) (e.g., a unique molecular identifier (UMI)), an index sequence, a sequence that is complementary to a sequencing primer (e.g., SBS12), or a combination thereof. In some embodiments, beads are packed onto a solid support (e.g., a planar support or flow cell), wherein the beads comprise a plurality of capture oligonucleotides immobilized thereon, wherein one or more of the plurality of capture oligonucleotides comprises, from 5′ to 3′: (a) a first clustering primer sequence; (b) a spatial barcode (SBC) sequence; (c) a first sequencing primer sequence; (d) a single molecule identifier (SMI) sequence; (e) a quality control sequence; and (f) a TVN sequence, wherein “T” is a capture nucleotide sequence, “V” is adenine (A), cytosine (C), or guanine (G), and “N” is adenine (A), cytosine (C), guanine (G), or thymine (T), and wherein the spatial barcode sequence of the plurality of capture oligonucleotides is unique to each bead

The oligonucleotides comprising a surface oligonucleotide (e.g., poly T sequences) can further comprise spatial index sequences, including, but not limited to, one or more of a P7 sequence, an index sequence, and/or a Read 2 (Rd2) sequence. In various embodiments, the surface oligonucleotide comprises a P7 anchor sequence, a spatial barcode and a sequence that hybridizes with a splint oligonucleotide.

In various embodiments, the sequence in the surface oligonucleotide that hybridizes with a splint oligonucleotide is a PZ (clustering) sequence. In various embodiments, the PZ sequence hybridizes to a splint oligonucleotide comprising a nucleotide sequence PZ′ complementary to the PZ sequence and a PX′ sequence that is complementary to the surface capture probe. In various embodiments, the PX sequence is a seeding sequence. In one embodiment, PX has the sequence

(SEQ ID NO: 21)
AGGAGGAGGAGGAGGAGGAGGAGG.

In various embodiments, the cleavable linker that attached a capture probe to the nanostructure is a cleavable polynucleotide. In various embodiments, the cleavable polynucleotide is between 5 to 25 nucleotides, or is 5, 6, 7, 8, 9, 10, 11, 12, 13, 14, 15, 16, 17, 18, 19, 20, 21, 22, 23, 24, or 25 nucleotides.

In some embodiments, the total RNA is released from the tissue sample. Release includes lysis of tissue or permeabilization of the tissue. In various embodiments, one or more samples that have been contacted with a solid support can be lysed to release target nucleic acids. Lysis can be carried out using known techniques, such as those that employ one or more of chemical treatment, enzymatic treatment, electroporation, heat, hypotonic treatment, sonication or the like. It is contemplated that the tissue sample is permeabilized prior capture. In various embodiments, the tissue sample is treated with one or more blocking reagents prior to capture. In various embodiments, the tissue sample is permeabilized and treated with one or more blocking reagents prior to capture.

In some embodiments, a tissue sample will be treated to remove embedding material (e.g., to remove paraffin or formalin) from the sample prior to release, capture or modification of nucleic acids. This can be achieved by contacting the sample with an appropriate solvent (e.g., xylene and ethanol washes). Treatment can occur prior to contacting the tissue sample with a solid support set forth herein or the treatment can occur while the tissue sample is on the solid support. Exemplary methods for manipulating tissues for use with solid supports to which nucleic acids are attached are set forth in US Pat. App. Publ. No. 2014/0066318, which is incorporated herein by reference.

A formalin-fixed tissue sample may also be decrosslinked using known techniques. In various embodiments, decrosslinking is carried out using Tris-EDTA (TE) buffer, e.g., at pH 8, pH 9, or another appropriate buffer at an appropriate pH. Decrosslinking may also be carried out at high heat, e.g., 70° C.

RNA from the sample may also be prepared by performing end repair of the RNA with polynucleotide kinase prior to the step of capturing RNA from the tissue sample, and/or by performing in situ polyadenylation with polyadenylate polymerase. prior to the step of capturing RNA from the tissue sample. Methods of carrying out end repair of RNA from a tissue sample are described in co-owned U.S. Provisional Application No. 63/477,730 (Docket No. 33080/IP-2625-P) (herein incorporated by reference).

The methods above are also useful for improving capture efficiency of mRNA transcripts for in situ mRNA transcript library preparation, and/or for improving the nucleotide length of polynucleotides used in generating an in situ transcriptome library (e.g., improving the polynucleotide size of cDNA transcribed from mRNA isolated from a sample and used in generating an in situ transcriptome library).

### Spatial Detection and Analysis of Nucleic Acids in a Tissue Sample

According to the methods described herein, spatial detection and analysis of nucleic acids in a tissue sample can be performed using sets of two or more capture probes (e.g., 3 or more, 4 or more, 5 or more, 6 or more, 7 or more, 8 or more, 9 or more, or 10 or more capture probes). Typically at least a first capture probe in a set of capture probes is immobilized on a capture array. In some embodiments, a second capture probe can be immobilized on the same capture array as the first capture probe, e.g., in proximity to the first capture probe, e.g., in the same capture site. In some embodiments, a second capture probe can be immobilized on a particle, such as a magnetic particle or a magnetic nanoparticle. In some embodiments, a second capture probe can be in solution, e.g., to be used to perform in situ reactions with a nucleic acid in a tissue sample.

Typically, at least a first capture probe in a set of capture probes is immobilized on a capture array or a nanostructure. In some embodiments, a second capture probe can be immobilized on the same capture array as the first capture probe, e.g., in proximity to the first capture probe, e.g., in the same capture site. In some embodiments, a second capture probe can be immobilized on a nanostructure or a particle, such as a magnetic particle or a magnetic nanoparticle. In some embodiments, a second capture probe can be in solution, e.g., to be used to perform in situ reactions with a nucleic acid in a tissue sample.

The capture probes in the capture probe sets individually and independently can have a variety of different regions, e.g., a capture region (e.g., a first universal or gene-specific capture region or first clustering region), a primer binding region (e.g., a SBS primer region, such as a SBS3 or SBS12 region), or a second universal region/clustering sequence, such as a P5 or P7 region, a spatial address region (e.g., a partial or combinatorial spatial address region), or a cleavable region.

“Sequencing-by-synthesis (“SBS”) techniques” generally involve the enzymatic extension of a nascent nucleic acid strand through the iterative addition of nucleotides against a template strand. In traditional methods of SBS, a single nucleotide monomer can be provided to a target nucleotide in the presence of a polymerase in each delivery. However, in the methods described herein, more than one type of nucleotide monomer can be provided to a target nucleic acid in the presence of a polymerase in a delivery.

Briefly, SBS can be initiated by contacting the barcodes with one or more labeled nucleotides, DNA polymerase, etc. Those features where a primer is extended using the sequences comprising the barcode as a template will incorporate a labeled nucleotide that can be detected. Optionally, the labeled nucleotides can further include a reversible termination property that terminates further primer extension once a nucleotide has been added to a primer. For example, a nucleotide analog having a reversible terminator moiety can be added to a primer such that subsequent extension cannot occur until a deblocking agent is delivered to remove the moiety. Thus, for embodiments that use reversible termination, a deblocking reagent can be delivered to the flow cell (before or after detection occurs). Washes can be carried out between the various delivery steps. The cycle can then be repeated n times to extend the primer by n nucleotides, thereby detecting a sequence of length n. Exemplary SBS procedures, fluidic systems and detection platforms that can be readily adapted for use with an array produced by the methods of the present disclosure are described, for example, in Bentley et al., Nature 456:53-59 (2008), WO 04/018497; WO 91/06678; WO 07/123744; U.S. Pat. Nos. 7,057,026; 7,329,492; 7,211,414; 7,315,019 or 7,405,281, and US Pat. App. Pub. No. 2008/0108082 A1, each of which is incorporated herein by reference.

Exemplary sequences include the following Rd1 and Rd2 adaptor sequences.

Second Universal Adapter -
Rd1 SBS3 (long):
(SEQ ID NO: 13)
ACACTCTTTCCCTACACGACGCTCTTCCGATCT;
Second Universal Adapter -
Rd1 SBS3 (short):
(SEQ ID NO: 14)
ACACTCTTTCCCTACACGAC;
First Universal Adapter -
Rd2 SBS12 (long):
(SEQ ID NO: 15)
GTGACTGGAGTTCAGACGTGTGCTCTTCCGATCT;
First Universal Adapter -
Rd2 SBS12 (short):
(SEQ ID NO: 16)
GTGACTGGAGTTCAGACGTGT.

In some embodiments, only one capture probe in a set of capture probes comprises a capture region. In some embodiments, two or more capture probes in a set of capture probes comprise as capture region.

In some embodiments, only one probe in a set of capture probes comprises a spatial address region, e.g., such as a complete spatial address region describing the position of a capture site on a capture array. In some embodiments, two or more probes in a set of capture probes can comprise a spatial address region, e.g., two or more probes can each comprise a partial spatial address region (i.e., combinatorial address region), wherein each partial address region describes the position of a capture site on a capture array, e.g., along the x-axis or the y-axis.

In some embodiments, a set of capture probes (e.g., a first and second capture probe) can comprise at least one capture probe comprising a capture region and a spatial address region (e.g., a complete or a partial spatial address region). In some embodiments, no capture probe in a set of capture probes comprises both a capture region and a spatial address region.

In some embodiments, the first capture probe is a 5′ gene specific probe comprising a sequence complementary to the first universal adapter sequence and a 5′ gene specific primer. In some embodiments, the RNA capture probe is a 5′ gene specific or target-specific probe comprising a sequence complementary to the first universal adapter sequence and a 5′ gene specific or target-specific primer.

In some embodiments, the second capture probe is a 3′ gene specific probe comprising a 3′ gene specific primer, a unique molecular index (UMI), and a second universal adapter sequence (e.g., Rd1 adapter). In some embodiments, the second capture probe does not comprise a spatial address region. In some embodiments, the surface capture probe is a 3′ gene specific or target-specific probe comprising a 3′ gene specific or target-specific primer, a unique molecular index (UMI), and a second universal adapter sequence (e.g., Rd1 adapter). In some embodiments, the surface capture probe does not comprise a spatial address region.

When surface oligonucleotide molecules are arranged randomly on the substrate (e.g., a flow cell), the method further comprises determining the substrate location of one or more surface oligonucleotide molecules by sequencing the spatial barcodes of the surface oligonucleotide molecules and assigning the spatial barcode sequences to locations on the substrate. Optionally, the In some embodiments, the RNA capture probe is a 5′ gene specific or target-specific probe comprising a sequence complementary to the first universal adapter sequence and a 5′ gene specific or target-specific primer. method further comprises sequencing at least a portion of one or more spatially barcoded first strand cDNA molecules, or copies thereof, to identify the spatial barcode sequences of the one or more spatially barcoded first strand cDNA molecules, or copies thereof, and correlating the spatial barcode sequences of the one or more spatially barcoded first strand cDNA molecules, or copies thereof, with the known locations of spatial barcode sequences of the surface oligonucleotide molecules. In various embodiments, the sequence of the spatial barcodes is determined by next generation sequencing.

When surface oligonucleotide molecules are arranged in clusters on the substrate (e.g., a flow cell), the method further comprises, prior to contacting the tissue sample with the substrate, determining the substrate location of each cluster by sequencing the spatial barcode for at least one surface oligonucleotide molecule in each cluster and assigning the spatial barcode sequence to a location on the substrate. Optionally, the method further comprises determining the spatial location of the RNA molecules within the tissue sample by sequencing at least a portion of one or more spatially barcoded first strand cDNA molecules, or copies thereof, to identify the spatial barcode sequences of the one or more spatially barcoded first strand cDNA molecules, or copies thereof, and correlating the spatial barcode sequences of the one or more spatially barcoded first strand cDNA molecules, or copies thereof, with the known locations of spatial barcode sequences of the surface oligonucleotide molecules. In various embodiments, the sequence of the spatial barcodes is determined by next generation sequencing.

When surface oligonucleotide molecules are arranged in a pattern on the substrate (e.g., a flow cell), such that the substrate locations and sequences of the spatial barcodes of the surface oligonucleotides on the substrate are known prior to contacting the tissue with the flow cell, the method further comprises determining the spatial location of the RNA molecules within the tissue sample by sequencing at least a portion of one or more spatially barcoded first strand cDNA molecules, or copies thereof, to identify the spatial barcode sequences of the spatially barcoded first strand cDNA molecules, or copies thereof, and correlating the spatial barcode sequences of the spatially barcoded first strand cDNA molecules, or copies thereof, with the known locations of spatial barcode sequences of the surface oligonucleotide molecules. Optionally, the method further comprises determining the spatial locations of RNA molecules within the tissue sample by sequencing at least a portion of one or more spatially barcoded first strand cDNA molecules and correlating the spatial barcode sequences of the one or more spatially barcoded first strand cDNA molecules, or copies thereof, with one or more corresponding spatial barcode sequences of surface oligonucleotide molecules having predetermined locations on the substrate.

In some embodiments, the capture site on the substrate is a plurality of capture sites. In some embodiments, the plurality of capture sites is 2 or more, 10 or more, 30 or more, 100 or more, 300 or more, 1,000 or more, 3,000 or more, 10,000 or more, 30,000 or more, 100,000 or more, 300,000 or more, 1,000,000 or more 3,000,000 or more, or 10,000,000 or 1,000,000,000 or more capture sites.

In various embodiments, the capture array or substrate comprises a capture site density of 1 or more, 2 or more, 10 or more, 30 or more, 100 or more, 300 or more, 1,000 or more, 3,000 or more, 10,000 or more, 100,000 or more, 1,000,000 or more, capture sites per square centimeter (cm²). In various embodiments, the density is between about 100k/mm² to about 1000k/mm², e.g., about 100k clusters/mm², about 200k clusters/mm², about 300 k clusters/mm², about 400k clusters/mm², about 500k clusters/mm², about 600k clusters/mm², about 700k clusters/mm², about 800k clusters/mm², about 900k clusters/mm², or about 1000k clusters/mm².

In various embodiments, the pair of capture probes in a capture site is a plurality of pairs of capture probes. In some embodiments, the plurality of capture probes is 2 or more, 10 or more, 30 or more, 100 or more, 300 or more, 1,000 or more, 3,000 or more, 10,000 or more, 30,000 or more, 100,000 or more, 300,000 or more, 1,000,000 or more 3,000,000 or more, or 10,000,000 or more, 100,000,000 or more, or 1,000,000,000 or more capture probes.

In some embodiments, the pair of capture probes in a capture site of a substrate is a plurality of pairs of capture probes. In some embodiments, each first capture probe in the plurality of pairs of capture probes within the same capture site comprises the same spatial address sequence. In some embodiments, each first capture probe in the plurality of pairs of capture probes in different capture sites comprises a different spatial address sequence.

In some embodiments, the surface of the capture array is a planar surface, e.g., a glass surface. In some embodiments, the surface of the capture array comprises one or more wells. In some embodiments, the one or more wells correspond to one or more capture sites. In some embodiments, the surface of the capture array is a bead surface.

In some embodiments, the capture region in the second capture probe is a gene-specific capture region. In some embodiments, the gene-specific capture region in the second capture probe comprises the sequence of a TruSeq™ Custom Amplicon (TSCA) oligonucleotide probe (Illumina, Inc.). For example, the gene-specific capture regions in a plurality of second capture probes in a capture site can comprise a plurality of sequences of TSCA oligonucleotide probes.

In some embodiments, the capture region in the second capture probe is a gene-specific or target specific capture region. In some embodiments, the gene-specific or target specific capture region in the second capture probe comprises the sequence of a TruSeq™ Custom Amplicon (TSCA) oligonucleotide probe (Illumina, Inc.). For example, the gene-specific or target-specific capture regions in a plurality of surface capture probes in a capture site can comprise a plurality of sequences of TSCA oligonucleotide probes.

Preparation of mRNA Library

The disclosure provides improved methods for preparing a mRNA transcript library from a sample providing a more complete spatial transcriptomics profile. The genetic profile of the sample may be used to diagnose and determine treatment for a subject having or at risk of having a disease as determined by the genetic profile.

Contemplated herein is a method of preparing a mRNA transcript expression library from a tissue sample, e.g., a fixed tissue sample, comprising a) mounting the tissue sample on a substrate comprising a plurality of capture oligonucleotides, wherein the capture oligonucleotides comprise a first clustering sequence (e.g., P7), a spatial barcode sequence (SBC) and a first universal adapter sequence (e.g., Rd2 adapter); b) contacting the tissue sample with i) a plurality of 5′ gene specific probes comprising a sequence complementary to the first universal adapter sequence and a 5′ gene specific primer; and ii) a plurality of 3′ gene specific probes comprising a 3′ gene specific primer, a unique molecular index, and a second universal adapter sequence (e.g., Rd1 adapter), under conditions such that one or more 5′ gene specific probe and one or more 3′ gene specific probe hybridizes to one or more mRNA transcript in the tissue sample; c) contacting the tissue sample in (b) with ligation reagents such that a 5′ gene specific probe and a 3′ gene specific probe hybridized to the mRNA transcript in proximity to each other are ligated together to form one or more ligated gene specific probe pairs; d) removing the mRNA transcript hybridized to the ligated gene specific probe pairs and leaving a ligated gene specific probe pair oligonucleotide sequence; and e) capturing the ligated gene specific probe pair oligonucleotide of (d) on the substrate by binding of the sequence complementary to the first universal adapter sequence in the 5′ gene specific probe to the first universal adapter sequence of the capture oligonucleotide (e.g., Rd2 adapter).

In various embodiments, the substrate is a glass slide, bead or flow cell. In various embodiments, the flow cell is an ordered flow cell or a random flow cell.

In various embodiments, the 5′ gene specific probes and/or 3′ gene specific probes are from 10-50 nucleotides in length, or from 20-40 nucleotides in length, or 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides.

In various embodiments the 3′ gene specific probe comprises one or more ribobases. In some embodiments, the 3′ gene specific probe comprises 1, 2, 3, 4, 5, or more ribobases.

In various embodiments, the UMI comprises from 6-20 nucleotides, or 6, 7, 8, 9, 10, 11, 12, 13, 14, 15, 16, 17, 18, 19 or 20 nucleotides.

In another embodiment, the method comprises hybridization of the transcripts as above in step (a) but wherein the hybridization leaves a nucleotide gap between the hybridized probes. Contemplated herein is a method wherein step (b) comprises contacting the tissue sample with i) a plurality of 5′ gene specific probes comprising a sequence complementary to the first universal adapter sequence and a 5′ gene specific primer; and ii) a plurality of 3′ gene specific probes comprising a 3′ gene specific primer, a unique molecular index, and a second universal adapter sequence (e.g., Rd1 adapter), under conditions such that one or more 5′ gene specific probe and one or more 3′ gene specific probe hybridizes to one or more mRNA transcript in the tissue sample, wherein hybridization of the 5′ gene specific probe and 3′ gene specific probe on the mRNA transcript results in a nucleotide gap between the hybridized molecules; and c) contacting the tissue sample in (b) with nucleotide bases and ligation reagents such that the nucleotide gap between the 5 gene specific probe and 3′ gene specific probe hybridized to the mRNA transcript is filled with nucleotide bases complementary to the mRNA transcript, and a 5′ gene specific probe and a 3′ gene specific probe are ligated together to form one or more ligated gene specific probe pairs. Steps (d) and (e) are similar to the above.

For the gap-fill reaction, the gap may be from 1-50 or more nucleotides, e.g., 50 or more nucleotides, from 1-50 nucleotides, from 1-40 nucleotides, from 1-30 nucleotides, from 1-20 nucleotides or from 1-10 nucleotides.

In various embodiments, the 5′ gene specific probes and/or 3′ gene specific probes comprise locked nucleic acids (LNA) in order to reduce or prevent strand displacement.

The clustering sequence can be a known index sequence. For example, in some embodiments, the first clustering sequence comprises a P7 sequence (e.g., CAAGCAGAAGACGGCATACG (SEQ ID NO: 3) or CAAGCAGAAGACGGCATACGAGAT (SEQ ID NO: 4) and the second clustering sequence comprises a P5 sequence (e.g., AATGATACGGCGACCACCGA (SEQ ID NO: 1) or AATGATACGGCGACCACCGAGATCTACAC (SEQ ID NO: 2)).

The universal primers also include sequences known in the field of spatial transcriptomics. In some embodiments, the first universal primer sequence comprises GTGACTGGAGTTCAGACGTGTGCTCTTCCGATCT (SEQ ID NO: 19). In some embodiments the second universal primer sequence comprises a Rd1 sequence set out in

(SEQ ID NO: 20)
AGATCGGAAGAGCGTCGTGTAGGGAAAGAGTGT.

In order to prevent unintended, premature capture to the substrate, the 5′ gene specific probes and 3′ gene specific probes anneal at a different temperature compared to the capture probes. In various embodiments, the 5′ gene specific probe and/or the 3′ gene specific probe have a melting temperature (Tm) of about 50-55° C. In various embodiments, the capture oligonucleotides have a melting temperature (Tm) of about 40-42° C.

In view of the desired melting temperatures, it is contemplated that step (b) of the methods is carried out at approximately 50-55° C. It is further contemplated that step (e) is carried out at approximately 40-42° C.

For the ligation reaction, multiple reverse transcriptases (RT) or polymerases are useful in the method. In various embodiments, the polymerase is T4 DNA ligase, T4 RNA Ligase 2 (T4Rnl2), SplintR DNA ligase, E. coli DNA ligase or R2D LIGASE. In various embodiments, the ligation reaction is carried out at 37° C.

Prior to strand synthesis, the mRNA transcript can be removed from the reaction, e.g., by enzymatic digestion. In various embodiments, the mRNA is removed using RNase H or RNase A.

The methods herein further comprise indexing and sequencing the ligated gene specific probe pairs comprising, f) performing extension reactions and PCR on the oligonucleotide of (e) to yield a PCR template representative of one or more mRNA transcripts in the tissue sample; g) eluting the PCR template from the substrate; and h) carrying out an indexing PCR to generate a double stranded PCR product comprising the first strand PCR product and a second strand complementary to the first strand PCR product.

In various embodiments, the PCR template is eluted form the substrate using sodium hydroxide elution. In various embodiments, the eluted PCR templates are placed in a tube for mRNA transcript library preparation.

In various embodiments, the method further comprises sequencing the PCR product of (h) and determining the location of the mRNA transcript in the tissue based on the spatial barcode sequence of (a).

In various embodiments, the double stranded PCR product comprises a second clustering sequence (e.g., P5) on the second strand complementary to the first strand PCR product and an index sequence.

It is contemplated that the method herein provides information about the location/position and expression level of particular genes in the tissue sample. For example, in the method, contacting the tissue sample with the substrate enables correlation of a position of a capture site on the substrate with a position in the tissue sample, wherein the substrate comprises a plurality of capture sites comprising a plurality of capture probes immobilized on a surface, wherein the capture probes comprise a spatial address region.

The disclosure also provides improved methods for preparing a spatially barcoded RNA library from a tissue sample providing a more complete spatial transcriptomics profile. Previous methods of generating an RNA library form a tissue sample involve ligating probe pairs with the sample RNA and ligating the probes together, which provides little info about the RNA sequence itself. It is hypothesized herein that separating the steps of hybridization and extension ligation will provide more robust sequence information from the initial capture of the RNA from a sample.

Multiple methods are proposed for copying or ligating portions of the RNA with targeted probes which can then be captured on (and then linked to) a spatially barcoded substrate. The RNA comprises ribosomal RNA (rRNA), messenger RNA (mRNA), non-coding RNA (ncRNA), small nuclear RNA (snRNA), small nucleolar RNA (snoRNA), and/or microRNA (miRNA).

In various embodiments, the substrate is a bead, a bead array, a spotted array, a substrate comprising a plurality of wells, a flow cell (e.g., a clustered flow cell), clustered particles arranged on a surface of a chip, a film, or a plate (e.g., a multi-well plate). In various embodiments, the substrate is a gel coating located in or on a flow cell.

In various embodiments, the substrate comprises a plurality of nanowells or microwells.

In various embodiments, the RNA capture probe is selected from the group consisting of a poly-T sequence, a randomer, or a target-specific probe. In various embodiments, the target-specific probes comprise a plurality of different target-specific RNA capture probe sequences. In various embodiments, the RNA capture probe or surface capture probe is between 8 to 80 nucleotides. In certain embodiments, the RNA capture probe or surface probe is between 10 to 80 nucleotides, between 10 to 70 nucleotides, between 10 to 60 nucleotides, between 10 to 50 nucleotides, between 10 to 40 nucleotides, between 10 to 30 nucleotides, between 10 to 20 nucleotides, between 20 to 80 nucleotides, between 20 to 70 nucleotides, between 20 to 60 nucleotides, between 20 to 50 nucleotides, between 20 to 40 nucleotides, or is 8, 9, 10, 11, 12, 13, 14, 15, 16, 17, 18, 19, 20, 21, 22, 23, 24, 25, 26, 27, 28, 29, 30, 40, 50, 60, 70 or 80 nucleotides.

In various embodiments, the target specific probes and/or substrate specific probes are from 10-50 nucleotides in length, or from 20-40 nucleotides in length, or 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides.

In various embodiments, the UMI comprises from 6-20 nucleotides, or 6, 7, 8, 9, 10, 11, 12, 13, 14, 15, 16, 17, 18, 19 or 20 nucleotides.

If a clustering sequence is employed, the clustering sequence can be a known index sequence. For example, in some embodiments, the first clustering sequence comprises a P7 sequence (e.g., CAAGCAGAAGACGGCATACG (SEQ ID NO: 3) or CAAGCAGAAGACGGCATACGAGAT (SEQ ID NO: 4)) and the second clustering sequence comprises a P5 sequence (e.g., AATGATACGGCGACCACCGA (SEQ ID NO: 1) or AATGATACGGCGACCACCGAGATCTACAC (SEQ ID NO: 2)).

The universal primers also include sequences known in the field of spatial transcriptomics. In some embodiments, the first universal primer sequence comprises GTGACTGGAGTTCAGACGTGTGCTCTTCCGATCT (SEQ ID NO: 19). In some embodiments the second universal primer sequence comprises a Rd1 sequence set out in

(SEQ ID NO: 20)
AGATCGGAAGAGCGTCGTGTAGGGAAAGAGTGT.

Prior to strand synthesis, the RNA, e.g., mRNA transcript, can be removed from the reaction, e.g., by enzymatic digestion. In various embodiments, the RNA is removed using RNase H or RNase A.

The methods herein further comprise indexing and sequencing the ligated gene specific or target-specific probe pairs comprising, performing extension reactions and PCR on the oligonucleotide to yield a PCR template representative of one or more mRNA transcripts in the tissue sample; eluting the PCR template from the substrate; and carrying out an indexing PCR to generate a double stranded PCR product comprising the first strand PCR product and a second strand complementary to the first strand PCR product.

In various embodiments, the PCR template is eluted form the substrate using sodium hydroxide elution. In various embodiments, the eluted PCR templates are placed in a tube for mRNA transcript library preparation.

In various embodiments, the method further comprises sequencing the PCR product and determining the location of the mRNA transcript in the tissue based on the spatial barcode sequence.

In various embodiments, the double stranded PCR product comprises a second clustering sequence (e.g., P5) on the second strand complementary to the first strand PCR product and an index sequence.

It is contemplated that the method herein provides information about the location/position and expression level of particular genes in the tissue sample. For example, in the method, contacting the tissue sample with the substrate enables correlation of a position of a capture site on the substrate with a position in the tissue sample, wherein the substrate comprises a plurality of capture sites comprising a plurality of capture probes immobilized on a surface, wherein the capture probe comprises a spatial address region.

### Biological Sample and Methods of Use

The present methods are useful to determine genetic information or a genetic profile, i.e., levels of specific genes or gene expression, detecting mutations or defects in a gene or change in genetic markers, from a biological sample in order to help diagnose a person who has or is at risk of having a disease as well as determine efficacy of treatment. Genetic profile refers to the characteristic expression level of one or more genes/genetic markers in a sample. In the present disclosure, genetic profile may be measured before, during and/or after administration of therapeutic to treat a disease as described herein, and it can be determine if gene levels changed, e.g., increased or decreased, in association with a particular disease, condition or treatment regimen.

A biological sample for use in the methods is obtained from a subject. In various embodiments, the subject is a mammal, such as humans, non-human primates such as chimpanzees, other apes and monkey species, cattle, horses, sheep, goats, swine, rabbits, dogs, cats, rodents, rats, mice, guinea pigs, and the like. In various embodiments the subject is a human.

The sample can be derived from an organ or tissue, including for example, from: the musculoskeletal system such as muscle, bone, tendon or ligament; an organ of the digestive system such as salivary gland, pharynx, esophagus, stomach, small intestine, large intestine, liver, gallbladder or pancreas; the respiratory system such as larynx, trachea, bronchi, lungs or diaphragm; the urinary system such as kidney, ureter, bladder or urethra; a reproductive organ/tissue such as ovary, fallopian tube, uterus, vagina, placenta, testicle, epididymis, vas deferens, seminal vesicle, prostate, penis or scrotum; the endocrine system such as pituitary gland, pineal gland, thyroid gland, parathyroid gland, or adrenal gland; the circulatory system such as heart, artery, vein or capillary; the lymphatic system such as lymphatic vessel, lymph node, bone marrow, thymus or spleen; the central nervous system such as brain, brainstem, cerebellum, spinal cord, cranial nerve, or spinal nerve; the eye, ear, nose, or tongue; or the integument such as skin, subcutaneous tissue or mammary gland.

A sample from a human can be considered (or suspected) healthy or diseased when use in the methods. In some cases, two specimens can be used: a first being considered diseased and a second being considered as healthy (e.g., for use as a healthy control). Any of a variety of conditions can be evaluated, including but not limited to, an autoimmune disease, cancer, cystic fibrosis, aneuploidy, pathogenic infection, psychological condition, hepatitis, a metabolic disorder, diabetes, sexually transmitted disease, heart disease, stroke, cardiovascular disease, multiple sclerosis or muscular dystrophy. In various embodiments, the disease or condition is cancer, a genetic condition or condition associated with pathogens having identifiable genetic signatures.

It is contemplated that the methods herein are useful to detect changes in genetic material compared to control sample, or sample of the subject prior to onset of a disease, including mutations, deletions, insertions, single nucleotide polymorphisms (SNP), combinations thereof, and other changes in the genetic profile.

The methods are also useful to determine if initiation with a therapy, e.g., cancer therapy, in a subject is needed, the method comprising i) determining the genetic profile of a subject using the methods described herein; ii) determining if the genetic profile indicates the subject has a disease or condition; and iii) starting treatment for the disease or condition with an appropriate therapy.

### Sequencing Methods

The methods described herein can be used in conjunction with a variety of nucleic acid sequencing techniques. Particularly applicable techniques are those wherein nucleic acids are attached at fixed locations in an array such that their relative positions do not change and wherein the array is repeatedly imaged. Embodiments in which images are obtained in different color channels, for example, coinciding with different labels used to distinguish one nucleotide base type from another are particularly applicable. In some embodiments, the process to determine the nucleotide sequence of a target nucleic acid can be an automated process. Preferred embodiments include sequencing-by-synthesis (“SBS”) techniques.

SBS can utilize nucleotide monomers that have a terminator moiety or those that lack any terminator moieties. Methods utilizing nucleotide monomers lacking terminators include, for example, pyrosequencing and sequencing using γ-phosphate-labeled nucleotides, as set forth in further detail below. In methods using nucleotide monomers lacking terminators, the number of nucleotides added in each cycle is generally variable and dependent upon the template sequence and the mode of nucleotide delivery. For SBS techniques that utilize nucleotide monomers having a terminator moiety, the terminator can be effectively irreversible under the sequencing conditions used as is the case for traditional Sanger sequencing which utilizes dideoxynucleotides, or the terminator can be reversible as is the case for sequencing methods developed by Solexa (now Illumina, Inc.).

SBS techniques can utilize nucleotide monomers that have a label moiety or those that lack a label moiety. Accordingly, incorporation events can be detected based on a characteristic of the label, such as fluorescence of the label; a characteristic of the nucleotide monomer such as molecular weight or charge; a byproduct of incorporation of the nucleotide, such as release of pyrophosphate; or the like. In embodiments, where two or more different nucleotides are present in a sequencing reagent, the different nucleotides can be distinguishable from each other, or alternatively, the two or more different labels can be the indistinguishable under the detection techniques being used. For example, the different nucleotides present in a sequencing reagent can have different labels and they can be distinguished using appropriate optics as exemplified by the sequencing methods developed by Illumina, Inc.

In various embodiments, the technique is a pyrosequencing technique. Pyrosequencing detects the release of inorganic pyrophosphate (PPi) as particular nucleotides are incorporated into the nascent strand (Ronaghi, M., Karamohamed, S., Pettersson, B., Uhlen, M. and Nyren, P. (1996) “Real-time DNA sequencing using detection of pyrophosphate release.” Analytical Biochemistry 242 (1), 84-9; Ronaghi, M. (2001) “Pyrosequencing sheds light on DNA sequencing.” Genome Res. 11 (1), 3-11; Ronaghi, M., Uhlen, M. and Nyren, P. (1998) “A sequencing method based on real-time pyrophosphate.” Science 281 (5375), 363; U.S. Pat. Nos. 6,210,891; 6,258,568 and 6,274,320, the disclosures of which are incorporated herein by reference in their entireties). In pyrosequencing, released PPi can be detected by being immediately converted to adenosine triphosphate (ATP) by ATP sulfurylase, and the level of ATP generated is detected via luciferase-produced photons. The nucleic acids to be sequenced can be attached to features in an array and the array can be imaged to capture the chemiluminscent signals that are produced due to incorporation of a nucleotides at the features of the array. An image can be obtained after the array is treated with a particular nucleotide type (e.g., A, T, C or G). Images obtained after addition of each nucleotide type will differ with regard to which features in the array are detected. These differences in the image reflect the different sequence content of the features on the array. However, the relative locations of each feature will remain unchanged in the images. The images can be stored, processed and analyzed using the methods set forth herein. For example, images obtained after treatment of the array with each different nucleotide type can be handled in the same way as exemplified herein for images obtained from different detection channels for reversible terminator-based sequencing methods.

In another exemplary type of SBS, cycle sequencing is accomplished by stepwise addition of reversible terminator nucleotides containing, for example, a cleavable or photobleachable dye label as described, for example, in International Patent Pub. No. WO 04/018497 and U.S. Pat. No. 7,057,026, the disclosures of which are incorporated herein by reference. This approach is being commercialized by Illumina Inc., and is also described in International Patent Pub. No. WO 91/06678 and International Patent Pub. No. WO 07/123,744, each of which is incorporated herein by reference. The availability of fluorescently-labeled terminators in which both the termination can be reversed and the fluorescent label cleaved facilitates efficient cyclic reversible termination (CRT) sequencing. Polymerases can also be co-engineered to efficiently incorporate and extend from these modified nucleotides.

Preferably in reversible terminator-based sequencing embodiments, the labels do not substantially inhibit extension under SBS reaction conditions. However, the detection labels can be removable, for example, by cleavage or degradation. Images can be captured following incorporation of labels into arrayed nucleic acid features. In particular embodiments, each cycle involves simultaneous delivery of four different nucleotide types to the array and each nucleotide type has a spectrally distinct label. Four images can then be obtained, each using a detection channel that is selective for one of the four different labels. Alternatively, different nucleotide types can be added sequentially and an image of the array can be obtained between each addition step. In such embodiments each image will show nucleic acid features that have incorporated nucleotides of a particular type. Different features will be present or absent in the different images due the different sequence content of each feature. However, the relative position of the features will remain unchanged in the images. Images obtained from such reversible terminator-SB S methods can be stored, processed and analyzed as set forth herein. Following the image capture step, labels can be removed and reversible terminator moieties can be removed for subsequent cycles of nucleotide addition and detection. Removal of the labels after they have been detected in a particular cycle and prior to a subsequent cycle can provide the advantage of reducing background signal and crosstalk between cycles. Examples of useful labels and removal methods are set forth below.

In particular embodiments some or all of the nucleotide monomers can include reversible terminators. In such embodiments, reversible terminators/cleavable fluors can include fluor linked to the ribose moiety via a 3′ ester linkage (Metzker, Genome Res. 15:1767-1776 (2005), which is incorporated herein by reference). Other approaches have separated the terminator chemistry from the cleavage of the fluorescence label (Ruparel et al., Proc Natl Acad Sci USA 102:5932-7 (2005), which is incorporated herein by reference in its entirety). Ruparel et al described the development of reversible terminators that used a small 3′ allyl group to block extension, but could easily be deblocked by a short treatment with a palladium catalyst. The fluorophore was attached to the base via a photocleavable linker that could easily be cleaved by a 30 second exposure to long wavelength UV light. Thus, either disulfide reduction or photocleavage can be used as a cleavable linker. Another approach to reversible termination is the use of natural termination that ensues after placement of a bulky dye on a dNTP. The presence of a charged bulky dye on the dNTP can act as an effective terminator through steric and/or electrostatic hindrance. The presence of one incorporation event prevents further incorporations unless the dye is removed. Cleavage of the dye removes the fluor and effectively reverses the termination. Examples of modified nucleotides are also described in U.S. Pat. Nos. 7,427,673, and 7,057,026, the disclosures of which are incorporated herein by reference in their entireties.

Additional exemplary SBS systems and methods which can be utilized with the methods and systems described herein are described in U.S. Patent Pub. No. 2007/0166705, U.S. Patent Pub. No. 2006/0188901, U.S. Pat. No. 7,057,026, U.S. Patent Pub. No. 2006/0240439, U.S. U.S. Patent Pub. No. 2006/0281109, International Patent Pub. No. WO 05/065814, U.S. Patent Pub. No. 2005/0100900, International Patent Pub. No. WO 06/064199, International Patent Pub. No. WO 07/010,251, U.S. U.S. Patent Pub. No. 2012/0270305 and U.S. Patent Pub. No. 2013/0260372, the disclosures of which are incorporated herein by reference in their entireties.

### Kits

As an additional aspect, the disclosure includes kits which comprise one or more compounds or compositions packaged in a manner which facilitates their use to practice methods of the disclosure. In one embodiment, such a kit includes a compound or composition described herein, packaged in a container such as a sealed bottle or vessel, with a label affixed to the container or included in the package that describes use of the compound or composition in practicing the method. Preferably, the compound or composition is packaged in a unit dosage form. Preferably, the kit contains instructions that describes use of the compositions.

Kits and articles of manufacture are contemplated herein. Such kits can comprise a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. The containers can be formed from a variety of materials such as glass or plastic. For example, the container(s) can comprise one or more spatially addressable probes disclosed herein, optionally in a composition or in combination with another agent (e.g., an array, a beadchip) as disclosed herein. The container(s) optionally have a sterile access port (for example the container can be an intravenous solution bag or a vial having a stopper pierceable by a hypodermic injection needle). Such kits optionally comprise an identifying description or label or instructions relating to its use in the methods described herein.

A kit will typically comprise one or more additional containers, each with one or more of various materials (such as reagents, optionally in concentrated form, and/or devices) desirable from a commercial and user standpoint for use with the spatially addressable probes described herein. Non-limiting examples of such materials include, but are not limited to, buffers, diluents, filters, needles, syringes; carrier, package, container, vial and/or tube labels listing contents and/or instructions for use, and package inserts with instructions for use. A set of instructions will also typically be included.

A label can be on or associated with the container. A label can be on a container when letters, numbers or other characters forming the label are attached, molded or etched into the container itself, a label can be associated with a container when it is present within a receptacle or carrier that also holds the container, e.g., as a package insert. A label can be used to indicate that the contents are to be used for a specific spatial-omic applications. The label can also indicate directions for use of the contents, such as in the methods described herein.

Additional aspects and details of the disclosure will be apparent from the following examples, which are intended to be illustrative rather than limiting.

### EXAMPLES

### Example 1—In Situ Method for Capture of mRNA Transcripts

In order to improve the capture of mRNA transcripts from a fixed or frozen tissue sample, improved methods for capturing mRNA transcripts from a tissue sample were developed.

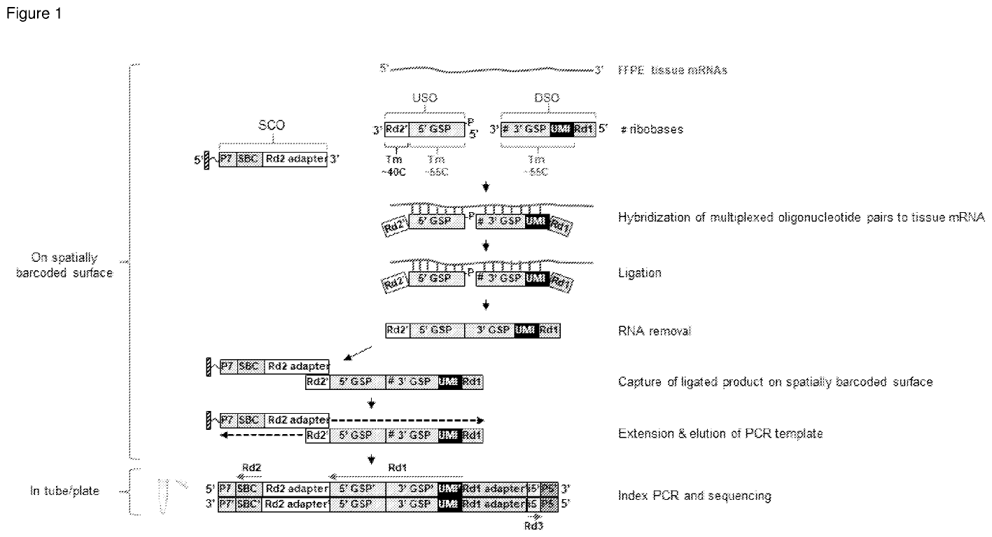

A schematic diagram of a first method is presented in FIG. 1. In the first method highly multiplexed oligonucleotide probes are hybridized to tissue mRNAs, followed by ligation, release and capture on a solid surface, comprising spatially barcoded capture oligonucleotides. Captured ligated products are eluted off the surface and PCR-amplified through universal adapter sequences to yield spatially barcoded libraries.

Two assay-specific oligonucleotides are designed to interrogate a single contiguous mRNA sequence (≤50 nt). Each of these oligonucleotides consists of two parts: an upstream-specific oligonucleotide (USO) containing a 5′ gene-specific sequence (5′ GSP), with a terminal phosphate, and a 3′ universal capture/partial Rd2′ adapter sequence (Rd2′), while the downstream-specific oligonucleotide (DSO) contains a 3′ gene-specific sequence (3′ GSP), followed by a unique molecular index (UMI) (N=6) and a 5′ Rd1 sequence (Rd1).) The GSPs of the USOs and DSOs are designed to have a Tm of approximately 55° C. each. Using this approach oligonucleotide pairs can be designed and multiplexed (pooled) to target the whole transcriptome. The spatially barcoded substrate contains covalently bound surface capture oligonucleotides (SCOs) containing a 5′ sequence for clustering (e.g., P7) followed by a spatial barcode (SBC) and a capture sequence (Rd2) complementary to the USO capture sequence. The SCO Rd2 sequence has a Tm of approximately 40° C.

Hybridization of the oligonucleotide pool occurs at a high temperature (approximately 50° C.) to favor GSP-mediated hybridization, but minimize USOs from hybridizing to capture oligonucleotide Rd2 sequences. Unbound 5′ and 3′ gene specific probes are removed via heated washes (approximately 50° C.). The 3′ terminus of the 3′ gene specific probe contains one or more ribobases to favor RNA ligase 2-mediated ligation. After RNA removal via RNase H and permeabilization, ligated cDNAs are captured on SCOs via Rd2′/Rd2 hybridization.

Extension from the 3′ end of the Rd2′-containing product releases the captured template from the surface, enabling indexed PCR off-surface (in solution) using an RNA tolerant PCR polymerase. For sequencing, Rd1 provides UMI and cDNA information, Rd2 yields spatial barcodes and Rd3 allows sample demultiplexing.

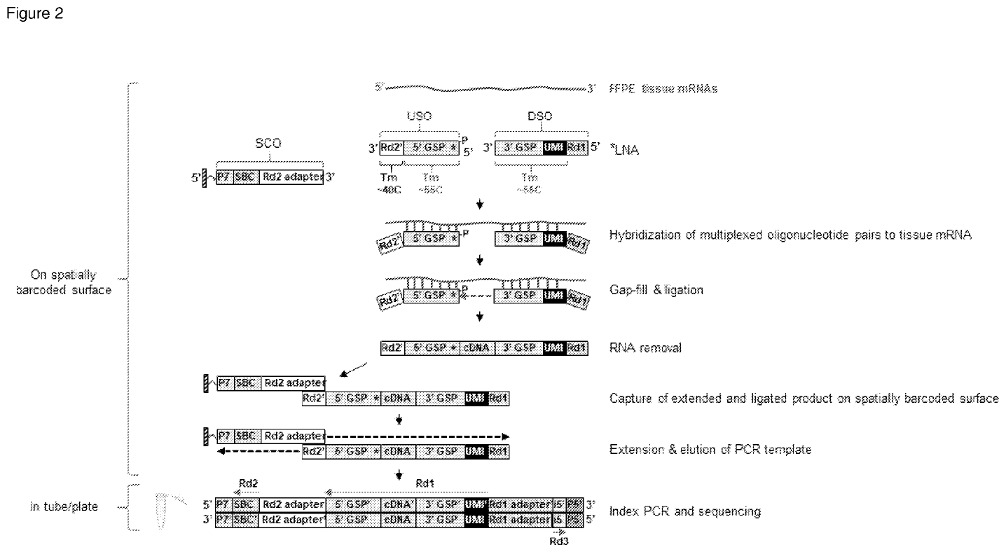

A schematic diagram of the second method is presented in FIG. 2. The second method is similar to that described in first method with the important distinction that sequences corresponding to the endogenous transcripts are captured, thereby affording additional assay specificity.

For the second method, GSPs of the 5′ and 3′ gene specific probes are designed to have a gap of a few nucleotides between the hybridized 3′ end of the 3′ gene specific probe and the 5′ end of the 5′ specific probe to provide additional assay specificity (the gap corresponds to endogenous mRNA sequence). Optionally, a polymerase possessing reverse transcriptase activity, but lacking strand-displacement activity is used for gap-filling, after which nicks are sealed with a ligase. The 5′ terminal bases of the 5′ gene specific probe contains a few locked nucleic acid bases (LNAs) to minimize any polymerase-derived strand-displacement activity. The subsequent steps are identical to those described in the first method, with the exception of the use of a LNA-tolerant PCR polymerase.

The workflow to develop the ability to isolate and prepare a mRNA library from formalin fixed, parafin-embedded (FFPE) tissue was developed herein. Each step of hybridization, ligation, surface capture and copy of transcript is performed on a substrate contianing the tissue sample. In order to minimize the off-target binding, initial hydrization of the probes to the target was done at approximately 55° C. using probes having a high melting temperature and that hybridize at that temepreature range. Once hydribization is completed, a wash at the same higher temperature was carried out and ligation (37° C.) performed. The capture reaction was performed at 40° C. The difference in reaction temperatures prevents the capture probe from hybrizing to the substrate surface too early and minimizes incomplete or premature capture of mRNA from the tissue sample.

Probe construction: As an initial step, probes were designed to produce a library using either the RNA-mediated oligonucleotide annealing, selection, and ligation with next-generation sequencing (RASL-Seq) method (Illumina), spatial annealing, selection, and ligation with next-generation sequencing (SPASL-Seq) or TruSeq method (Illumina).

Probes for a RASL-seq, which binds RNA, comprise an index primer at the 5′ end (Rd2 adapter) linked to a 3′ target sequence (gene specific probe) and a 5′ target sequence (gene specific probe) linked to a 3′ P5 primer. TruSeq primers, which bind cDNA, comprise 5′ smRNA linked to a USO and a DSO linked 3′ to a SBS3 sequence. Primers were designed to leave gaps or to leave no gaps once hybridized to the mRNA transcript. If primer hybridization left gaps between the probes on the target polynucleotide, an extension reaction was carried out to fill the gaps. Two library models, a human control library comprising genes expressed as low CV across cell types, and a ERCC control model, were used to assay the probes.

Probes were annealed to the target polynucleotide and ligated together to form a single strand complementary to the target polynucleotide. This was then captured on a substrate comprising poly T and sequences complementary to the 5′ end of the ligated product (Rd2 adapter) and extended via polynucleotide extension reaction. The ligated strand was then eluted from the capture surface and second strand synthesis carried out by PCR. The primer for second strand synthesis contained a Rd1 adapter sequence, an indexing sequence (e.g., i5) and a Rd3 sequence (e.g., P5). It was noted that use of 3′ ribobases in the upstream ligating oligonucleotide (USO) improves efficiency of ssDNA ligation in solution. The ribobases reduce strand displacement during the ligation reaction.

Probes for TruSeq methods were designed in a similar means to those for RASL-Seq above but comprise on the first probe a 5′ Rd2 sequence, a unique molecular identifier (UMI) and an ULSO and on the second probe a DLSO and a 3′ adapter sequence (Rd1). For the reaction, 2-probe subpool on ERCC (30 nM) was used, 1 ul or 0.1 ul of ERCC (3 nM or 0.3 nM in 10 ul mix) and the Titrate probe concentration titrated-ERCC pool 8012 50 uM, 5 uM, or 0.5 uM. Annealing conditions were 50 mM NaCl in IDTE at a Gradient of 65° C. for 5′, 45° C. for 5′, 37° C. for 10′, 25° C. for 10′. Results demonstrate that high probe concentration appears to inhibit qPCR.

Ligation Assay: A ligation assay for the particular methods was also designed in order to ligate oligonucleotides in situ on a substrate containing tissue. Several different ligases were assayed for efficiency in the reaction, T4 DNA ligase, T4 RNA Ligase 2 (T4Rnl2), SplintR DNA ligase, E. coli DNA ligase and R2D LIGASE™. 9× [enzyme] conditions performed best across conditions. T4 RNA Ligase 2 performance was similar to other enzymes at higher concentrations. R2D also appeared to have similar ligation efficiency.

During the ligation assay analysis, ribobases were added to the 3′ end of the DLSO to determine if this would improve ligation efficiency. Adding 3′ ribobases to the ligation assay increases ligation efficiency for T4 RNA ligase 2 in both single stranded and splinted reactions, but not for T4 DNA ligase, E coli DNA ligase or SplintR.

Prior to hybridization, it was tested if reversal of tissue sample cross-linking (e.g., a byproduct of formalin fixation of the sample) would improve hybridization and capture efficiency. Cross linking reversal was carried out under different conditions using commercial RNA extraction kits, RNAeasy or RNASTORM™. Fresh frozen (FF) or FFPE tissue sections were collected and if needed paraffin removed from the sample and tissue lysed using standard protocols. Different conditions for RNA extraction were used and quantity of RNA recovered determined. For Qiagen RNeasy (Qiagen): FF, FFPE (50 ul, 30 ul elution, respectively) included a 15 min reverse crosslink step in the FFPE kit. The time course used was 70° C. at 0, 15′, 30′, 45′, 1 h, 2 h, 4 h, O/N; 100 ng in 20 ul, or 0, 15′, 30′, 60′ at 80° C. during extraction. Under those conditions, the main increase in accessibility (RT-qPCR reduction in Cq) is from 0-30′ at 80° C. It appears that mRNA reduction correlates more strongly with RNA input into the reaction. RNASTORM™ (Cell Data Science) extraction was carried out at the same conditions as above and quantity of RNA recovered was determined. The reactions showed that there appears to be a slight increase in RNA recovery using the RNAeasy extraction, but additional experiments will be carried out to confirm.

TABLE 1

| | | Qiagen | Yield from 50 uL | Yield from 30 uL |
|---|---|---|---|---|
| | RNA Storm | Rneasy | elution RNA | elution Qiagen |
| Tubes | (ng\ul) | (ng\ul) | Storm (ng) | RNeasy (ng) |
| 1 | 32.8 | 48 | 1640 | 2400 |
| 2 | 28.2 | 114 | 1410 | 5700 |
| 3 | 17.2 | 176 | 860 | 8800 |
| 4 | 23.8 | 166 | 1190 | 8300 |
| 5 | 15 | 164 | 750 | 8200 |
| Pooled | 25 ng/uL | 122 ng/uL | 5850 ng | 33400 ng |

Note:
FFPE collection difficulties from high lab temp resulted in each tube containing 2-3.5 mouse kidney sections each

The results show that the present methods of capturing mRNA from FFPE tissue samples are effective to improve capture efficiency and transcript integrity thereby providing for a more robust spatial transcriptomics library. This improved library is useful to more distinctly characterize a genetic profile at the cellular and positional level, e.g., in a sample from a subject with a disease or condition, and help diagnose and treat such a disease or condition.

### Example 2—Methods for Generation of RNA Library

In order to improve the capture of RNA transcripts from a fixed or frozen tissue sample, improved methods for capturing RNA from a tissue sample were developed.

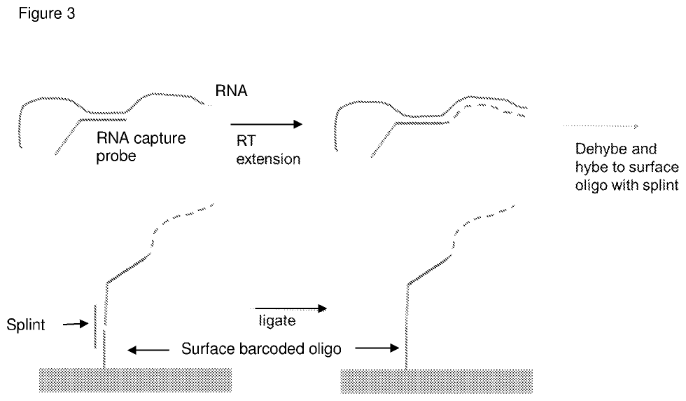

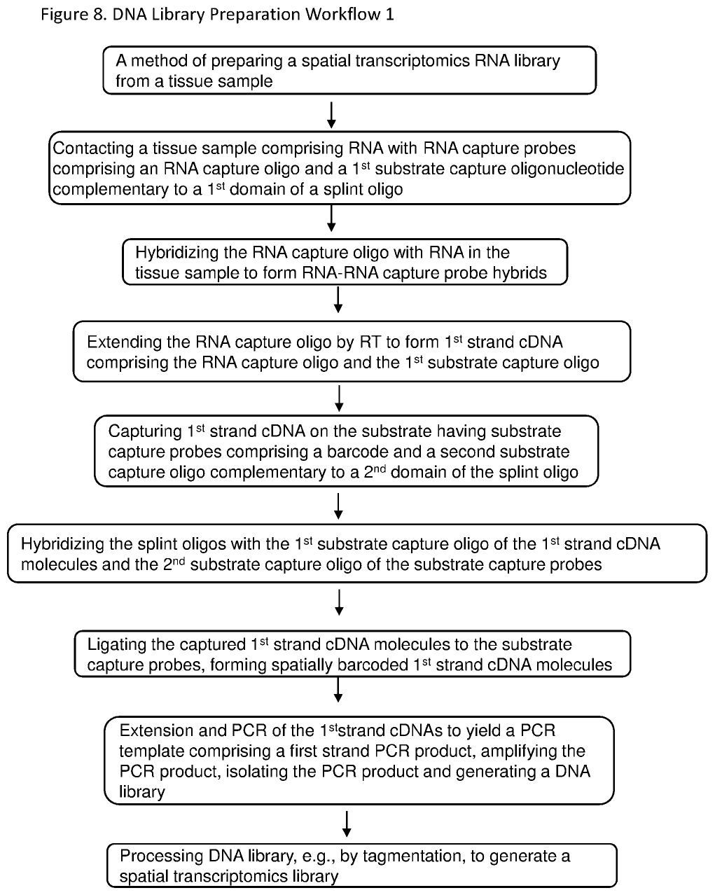

A schematic diagram of a first method is presented in FIG. 3 and an example workflow is illustrated in FIG. 8.

In a first exemplary method, RNA capture probes are hybridized with RNA in tissue, followed by extension with a reverse transcriptase to form a first strand cDNA molecule (FIG. 3). The RNA capture probes comprise a capture oligonucleotide sequence complementary to an RNA in the sample and a first substrate capture oligonucleotide that is complementary to a first domain of a plurality of splint oligonucleotides. In an optional step, the extended probe is then melted off the RNA (or RNA is digested with RNAse) and the probe is hybridized via a splint oligonucleotide to a surface barcoded oligonucleotide on a substrate. Substrate capture probes each comprise a spatial barcode and a second substrate capture oligonucleotide complementary to a second domain of the splint oligonucleotides. The captured first strand cDNA molecules are then ligated to the surface barcode oligonucleotide to the extended probe, e.g., using T4 ligase, to generate spatially barcoded first strand cDNA. The surface oligo can also contain an adaptor sequence, e.g., a P7 adaptor, and the RNA capture probe may also contain a read primer hybridization site for reading the spatial barcode. Optionally, instead of dehybridizing the extended probe, if the RNA is mobile (i.e., not crosslinked to tissue, or liberated by decrosslinking), then the entire construct could bind to the substrate surface oligo and be ligated followed by on-surface reverse transcription. Optionally, instead of dehybridizing the extended probe, the RNA could be digested, releasing the DNA probe. Ligation could be via enzymatic or chemical methods.

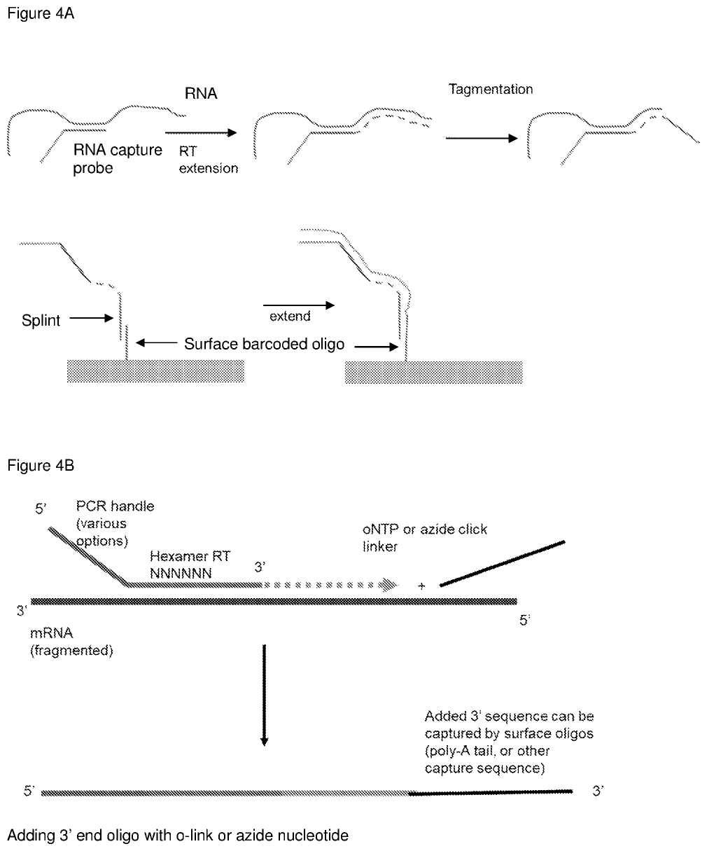

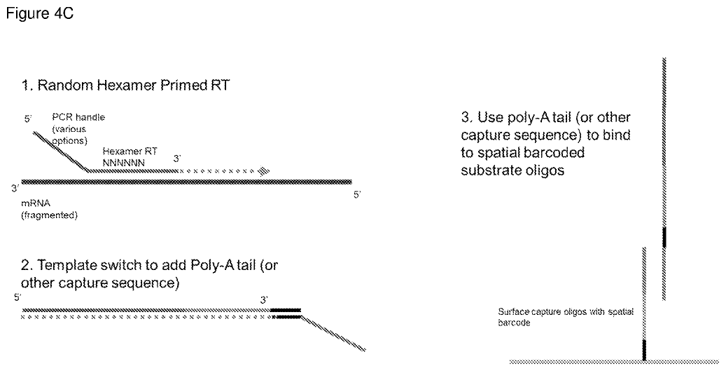

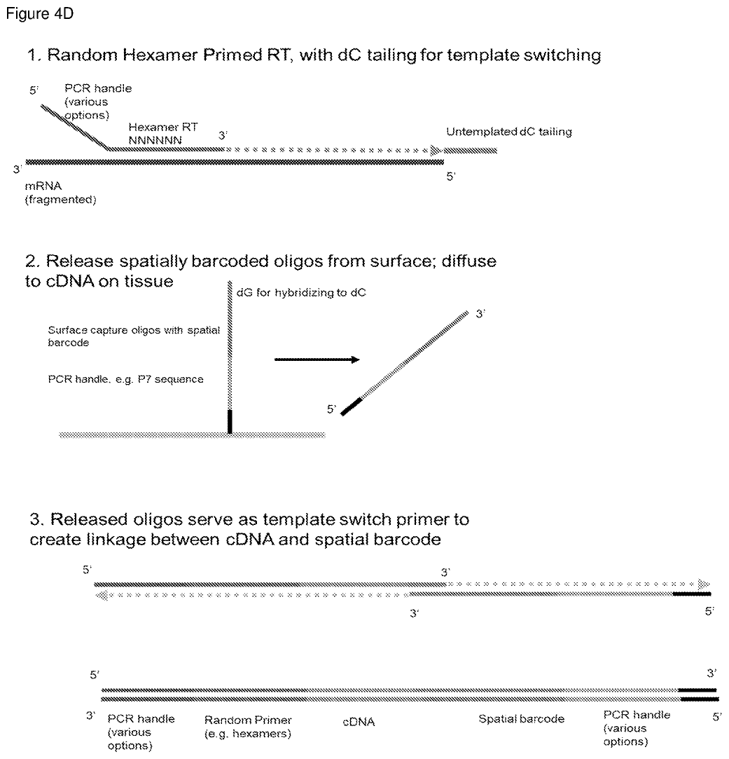

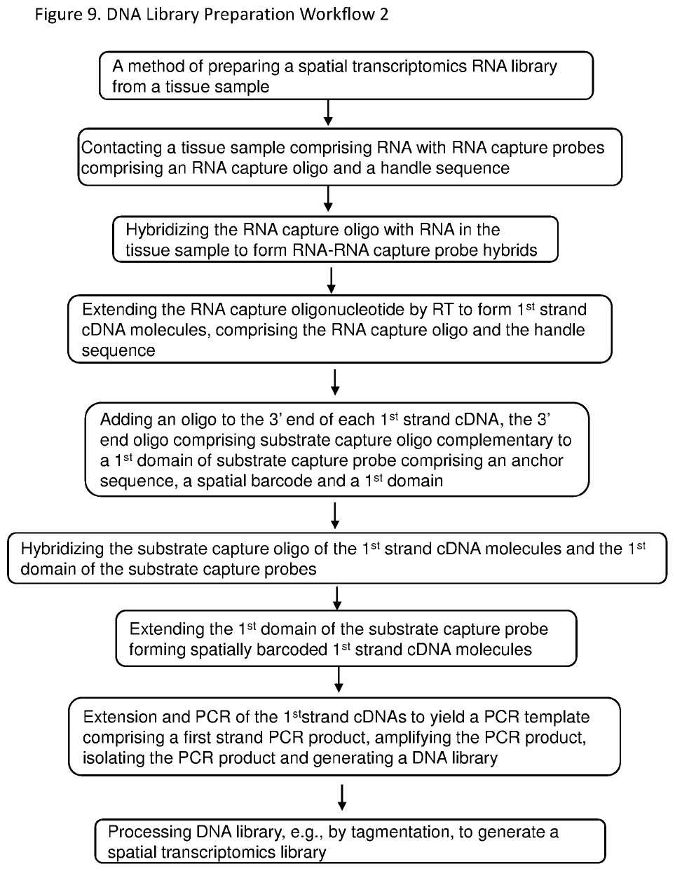

As a permutation of the above strategy, in a second approach an oligonucleotide is added to the 3′ end of the extended probe, which is complementary to a portion of the surface oligonucleotide (FIG. 4A, FIG. 9). In this method, the RNA capture probe comprises an oligonucleotide sequence complementary to the RNA in the sample and a handle sequence. Initially, the RNA capture oligonucleotide of the RNA capture probes are hybridized with RNA in the tissue sample to form RNA-RNA capture probe hybrids. The RNA-RNA hybrids are extended using RT to generate first strand cDNA. A 3′ oligonucleotide sequence comprising a substrate capture oligonucleotide complementary to a first domain of a substrate capture probe is then added to the first strand cDNA. The surface capture probes comprise in the 5′ to 3′ orientation, a substrate anchor sequence, a spatial barcode, and the first domain. The substrate capture oligonucleotide of the first strand cDNA molecules is then hybridized with the first domain of the substrate capture probes; extension of the first domain of the hybridized substrate capture probes is performed resulting in spatially barcoded first strand cDNA molecules.

This 3′ end oligonucleotide enables extension from the surface capture probe and then the 5′ end of the probe can be used to introduce the P5 or other adaptor. The method of adding the oligo to the 3′ end in FIG. 4A is shown as tagmentation Tn5 has some activity toward DNA/RNA hybrids and could be used for adding a 3′ OH by tagmentation (FIG. 4A). 3′ oligonucleotide addition could also be achieved by terminating the first extension step with a click labeled nucleotide (e.g., an azide or alkyne) or similarly, by oNTP-directed adapterization, followed by chemical ligation with the oppositely functionalized surface barcoded oligonucleotide (or sequence complementary thereto on the 3′ end of the cDNA transcript) (FIG. 4B).

It is contemplated that the added 3′ oligonucleotide sequence can be captured by surface oligos, e.g., a polyA tail or other capture sequences. These modified nucleotides (click and oNTP) could also be used to terminate the cDNA product to an appropriate insert length for sequencing. An alternate method is to polyadenylate the extended probe using TdT (or other mononucleotide addition), and bind this product to polyT at the 3′ end of the spatial barcode oligo.

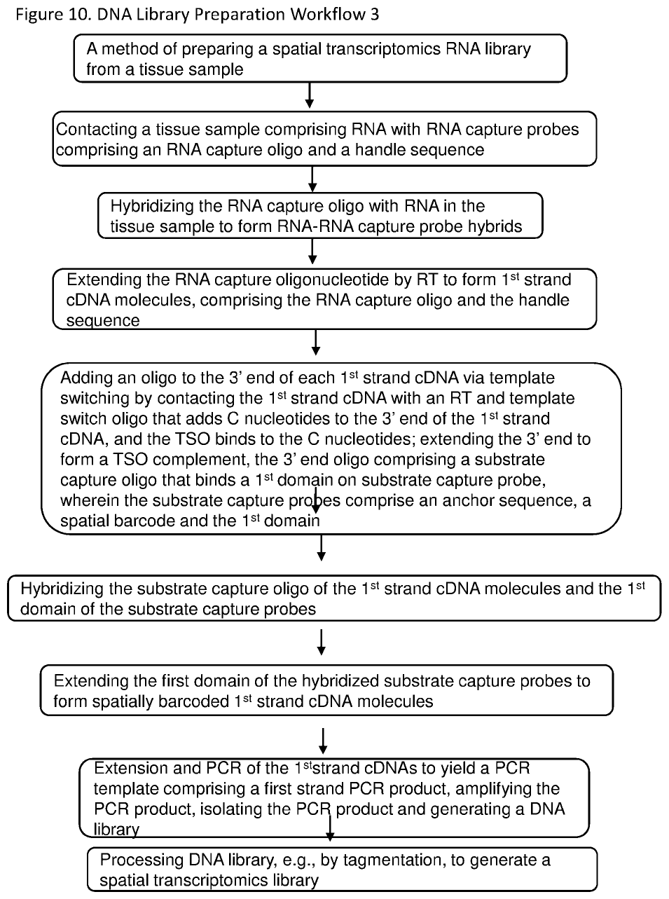

Template switching could be another method to add a poly-A tail or other capture sequence to the 3′ end of the first strand cDNA molecule (FIG. 4C, FIG. 10). Similar to the above methods, the RNA capture oligonucleotide of the RNA capture probes are hybridized with RNA in the tissue sample to form RNA-RNA capture probe hybrids. The RNA-RNA hybrids are extended using RT to generate first strand cDNA. To add the 3′ oligonucleotide, the first strand cDNA molecule is contacted with a reverse transcriptase (RT) and a template switch oligonucleotide (TSO), wherein the RT incorporates untemplated cytosine nucleotides at the 3′ end of the first cDNA and the TSO comprises a sequence capable of hybridizing to the untemplated cytosine nucleotides, and the RT extends to generate a TSO complement. In this example, the 3′ end oligonucleotide comprises a substrate capture oligonucleotide complementary to a first domain of a plurality of substrate capture probes on a substrate, and each of the plurality of substrate capture probes comprises, in the 5′ to 3′ orientation, a substrate anchor sequence, a spatial barcode, and the first domain.

Once the 3′ oligonucleotide is added by template switching, the 3′ end oligo is used to hybridize to the spatially barcoded surface oligo, followed by extension of the surface oligo to link the spatial barcode to the first strand cDNA sequence. In one variation, the DNA is dC tailed by reverse transcription and is used as the capture sequence, hybridizing to a dG end sequence on the spatially barcoded substrate capture oligo.

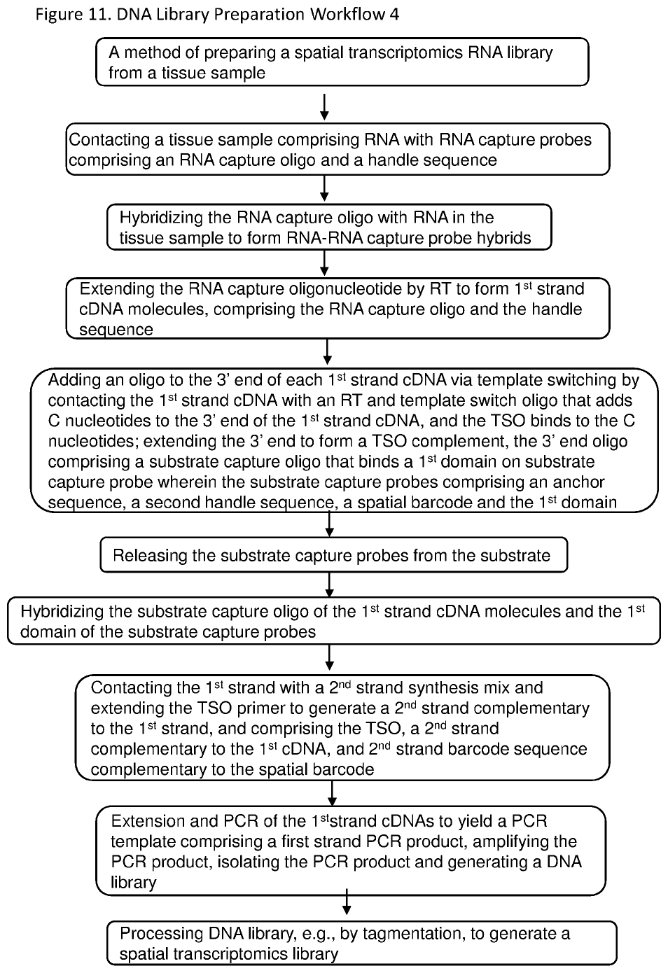

In another variation of template switching, the spatially barcoded oligos on the substrate surface can be released to serve as the template switch primer (FIG. 4D, FIG. 11). In this exemplary method, the 3′ end oligonucleotide can comprise a substrate capture oligonucleotide complementary to a first domain of a substrate capture probe on a substrate, wherein each of the substrate capture probes comprises, in the 5′ to 3′ orientation, a substrate anchor sequence, a second handle, a spatial barcode, and the first domain. Surface capture probes are released from the substrate and serve as a template switching primer which is then useful to spatially barcode the first strand cDNA. Releasing surface capture probes from the surface may be useful in spatially addressing tissues that are mounted on slides that do not have capture oligos on them.

Downstream library preparation steps on the ligated surface could include: random priming of a 2nd strand using a random primer that contains the P5 sequence or similar handle, ligation of P5 adaptor, polyadenylation via TdT followed by PCR to introduce the P5 adaptor, etc. The P5 end could also be introduced using template switching during the RT extension. SMIs could also be introduced during the library preparation via any of the above approaches.

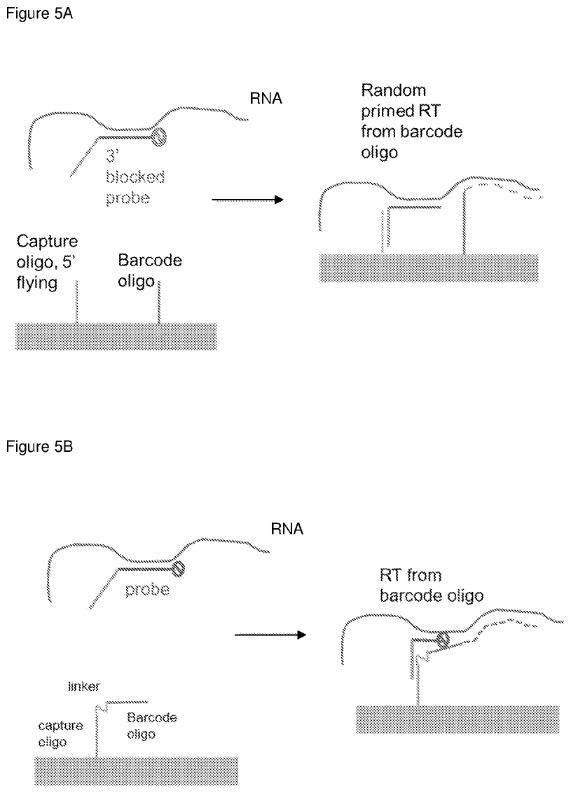

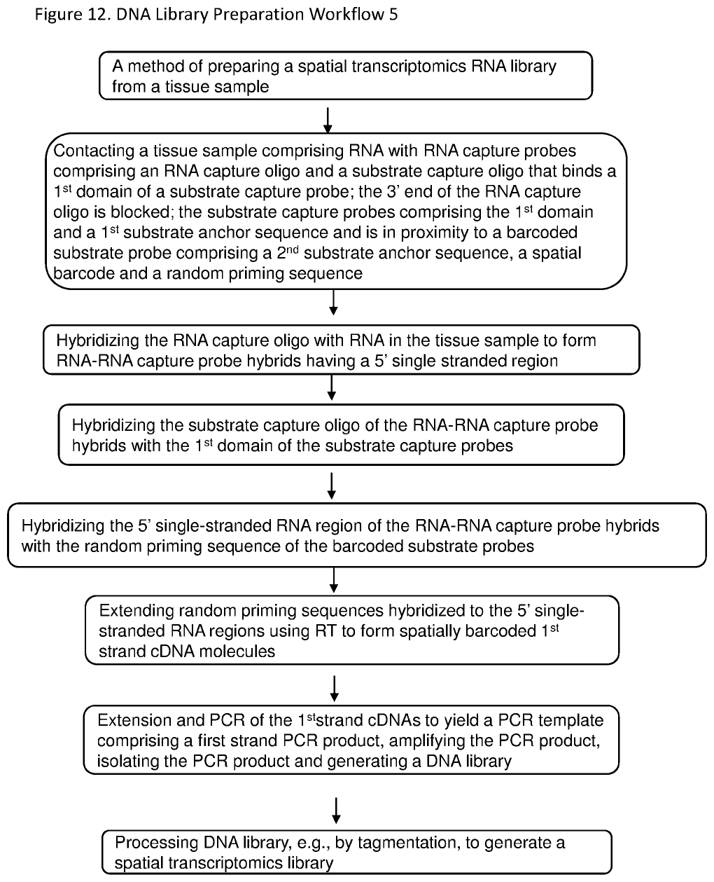

In another approach, random priming is carried out from pulled down RNA. RNA, e.g., mRNA, is bound down to the surface of a substrate via hybridization between a blocked probe (e.g., comprising a 3′ phosphate) and a surface capture oligo anchored to the substrate (shown with 5′ as the free end for hybridization, i.e., 5′ flying, but could be 3′ flying) (FIG. 5A, FIG. 12). A second, barcoded oligo (which is 5′ anchored) is also on the substrate in proximity to a substrate capture oligo. The RNA capture oligonucleotides having a 3′ OH blocked probe are hybridized with RNA in the tissue sample to form RNA-RNA capture probe hybrids having a 5′ single-stranded RNA region. The substrate capture oligonucleotide of the RNA-RNA capture probe hybridizes with the first domain of the substrate capture probes and the 5′ single-stranded RNA region of the RNA-RNA capture probe hybrids anneals with the random priming sequence of the barcoded substrate probes. Extension of the random priming sequences hybridized to the 5′ single-stranded RNA is carried out using RT to form spatially barcoded first strand cDNA molecules.

As seen from FIG. 5A, the barcoded oligo is used to randomly prime the RNA, thereby linking the spatial barcode with the RNA transcript. The barcode oligo would also contain P7 or other adapter and a barcode read primer site. Downstream library preparation steps as described above would be used to generate a second strand cDNA. Random priming of polyA mRNA could also be performed if the capture oligo were a polyT. This would enable copying a section of the mRNA that is distant from the polyA tail 3′ end (i.e., possibly within the coding region instead of 3′UTR). This is something that is lacking in standard polyA capture spatial approaches.

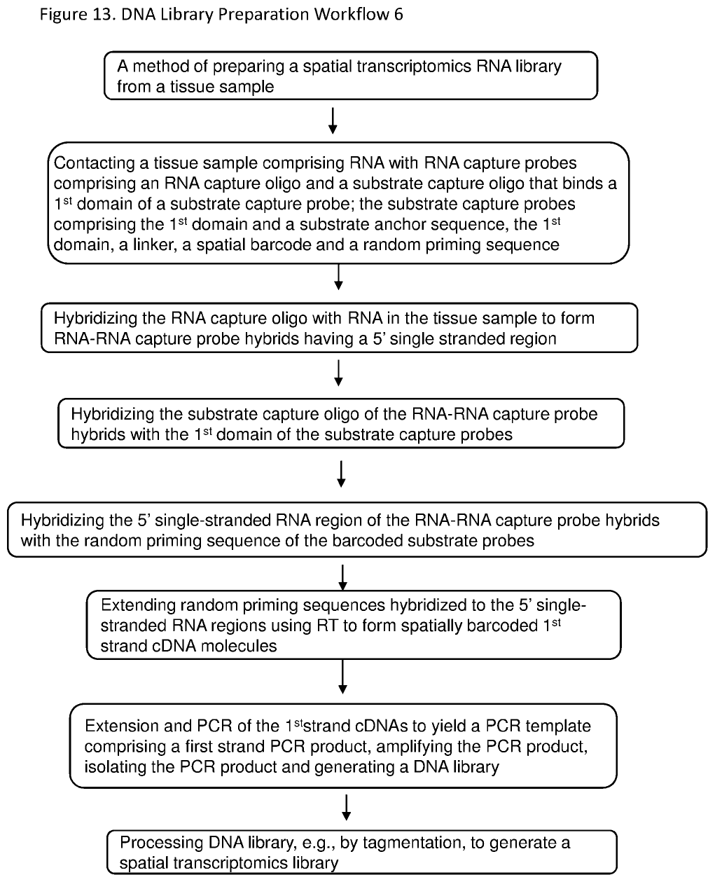

Another version of this scheme has the capture oligo and barcode oligo concatenated via linkers that cannot be read through by polymerase (FIG. 5B, FIG. 13). The advantage of this scheme is more space on the substrate surface would be available, allowing higher complexity probesets to be used to pull down RNA in the sample.

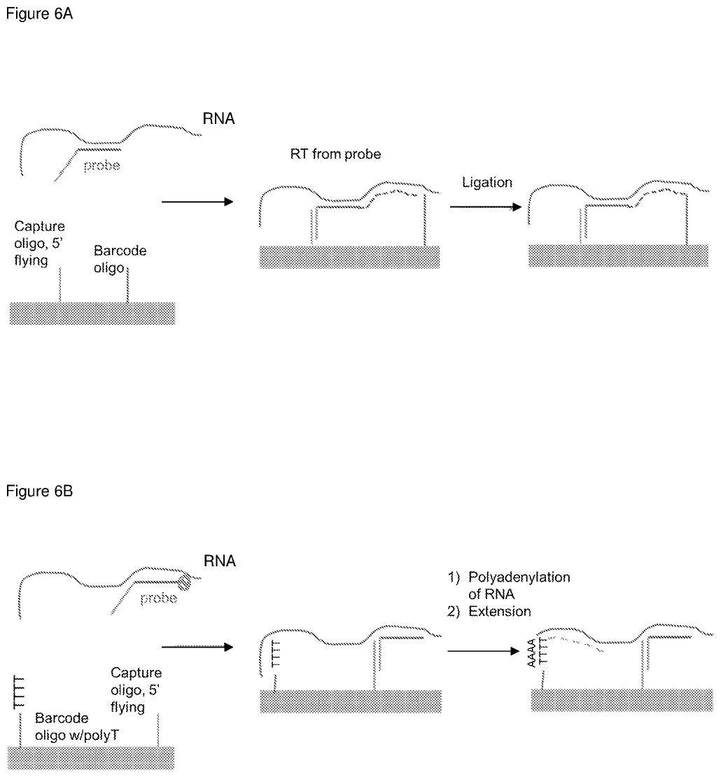

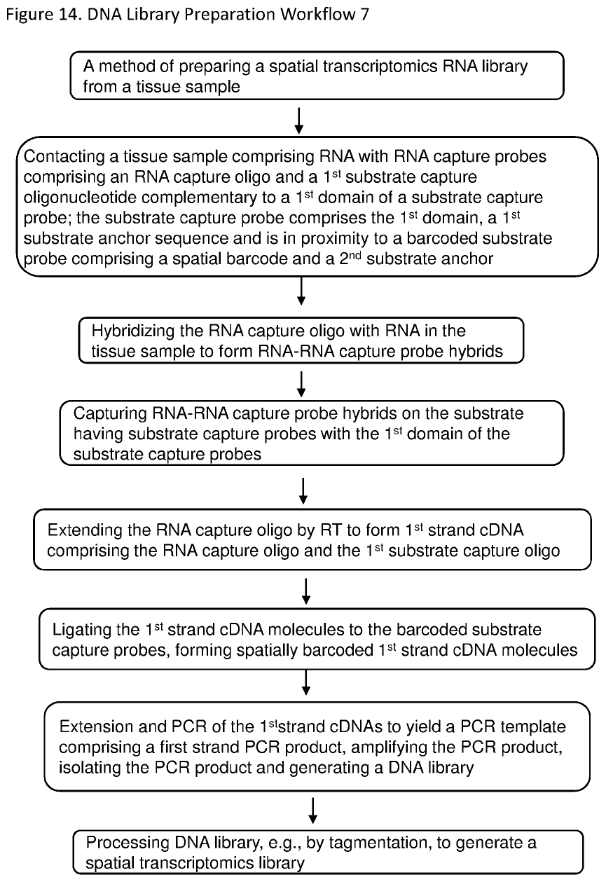

Probe extension followed by ligation to surface spatial barcode oligos is also contemplated as a method herein (FIG. 6A, FIG. 14). In this method, unblocked RNA capture probes comprising an RNA capture oligonucleotide complementary to an RNA in the sample and a substrate capture oligonucleotide complementary to a first domain of a plurality of substrate capture probes are used to bind RNA to the surface. The substrate capture probes can comprise, in the 5′ to 3′ orientation, the first domain and a first substrate anchor sequence, and are in proximity to a barcoded substrate probe on the substrate, comprising, in the 5′ to 3′ orientation, a spatial barcode and a second substrate anchor sequence. The RNA-RNA hybrids are also used to prime RT. For example, extension of the RNA capture oligonucleotide of the captured RNA-RNA capture probe hybrids is carried out using RT to form first strand cDNA molecules. The first strand cDNA is ligated to the spatial barcode oligo. The difference between this approach and the previous approach is that splint ligation is not needed because of concentration enhancement by forced localization to the ligation target and the P5 adaptor can be introduced in the 5′ appendage to the probe. Optionally, ligation can be carried out chemically using 3′ click nucleotides in the cDNA or via an oNTP incorporated at the 3′ end which acts as its own splint to ligate to the barcode oligo. Other chemical ligation methods such as 5′OH to 3′phos using EDC or oNTP incorporation followed by ligation could also be used.

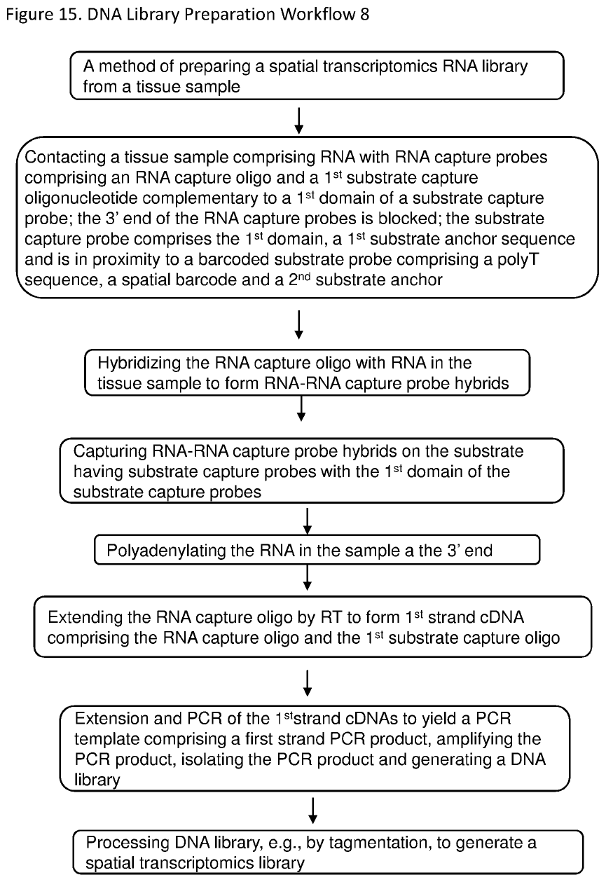

Another permutation of this method uses a blocked RNA capture probe and 3′ polyadenylation of the RNA (using PAP for instance) to enable polyA addition and extension (FIG. 6B, FIG. 15). The barcode oligonucleotide can comprise a polyT sequence to bind to polyA and the capture oligo on the substrate may be, but is not necessarily, 5′ flying. The RNA capture probe oligonucleotide binds to the capture probe and facilitates the ligation of the extended RT to the barcode oligonucleotide on the surface of the substrate.

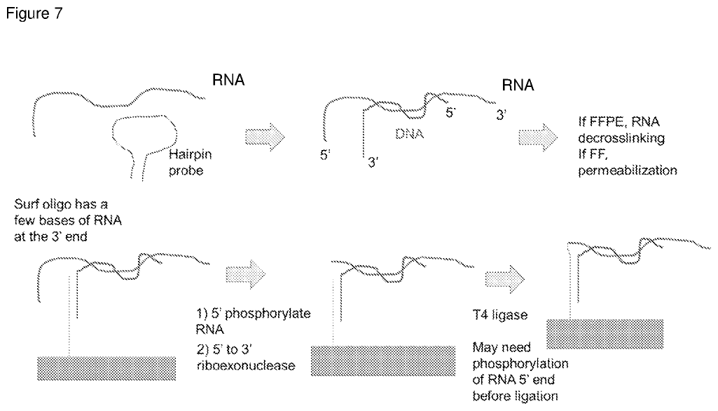

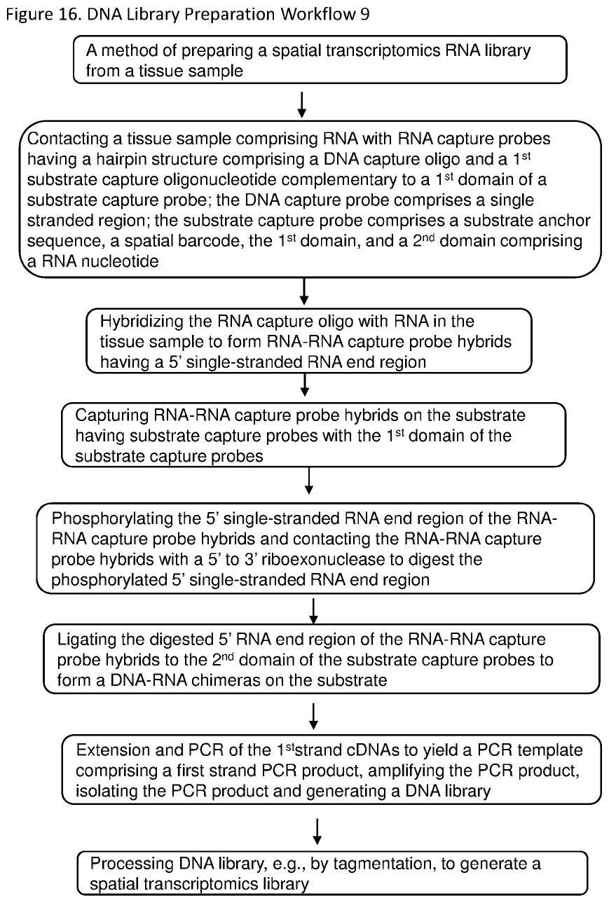

In an alternate method, direct ligation of RNA to surface spatial barcoded oligos is contemplated (FIG. 7, FIG. 16). In this method, RNA capture probes having a hairpin structure and comprising a DNA capture oligonucleotide complementary to RNA in the sample and a substrate capture oligonucleotide complementary to a first domain of a substrate capture probe are used. The DNA capture oligonucleotide comprises a single stranded region, and each of the substrate capture probes can comprise, in the 5′ to 3′ orientation, a substrate anchor sequence, a spatial barcode, the first domain, and a second domain, wherein the second domain comprises at least one RNA nucleotide or nucleoside. RNA-RNA capture probe hybrids are formed and each of the RNA-RNA capture probe hybrids comprises a 5′ single-stranded RNA end region. 5′ RNA can be ligated to 3′ DNA using T4 ligase. The RNA-RNA capture probe hybrids are captured by the substrate capture oligo in the substrate capture probe. RNA can be captured using a probe which also bind to the surface barcode oligo. Hairpin probes may prevent excess probes from occupying surface sites, which would otherwise require more stringent washes or higher Tm surface capture oligos. Next, the 5′ single stranded RNA can be 5′ phosphorylated, enabling a 5′ to 3′ riboexonuclease to digest the overhanging RNA. The digested 5′ RNA end region of the captured RNA-RNA capture probe hybrids is ligated, e.g., using T4 ligase, to the second domain of the substrate capture probes to DNA-RNA chimeras. The surface oligo may have a few ribobases at the 3′ end. The DNA-RNA chimera could be converted to DNA by reverse transcription using a DNA random primer, which could also contain P5. 3′ polyadenylation of the chimera could also be used to enable priming using polyT. If carried out on FFPE tissue, the RNA can be decrosslinked. If the method is carried out with fresh frozen tissue, the tissue is permeabilized to release RNA.

It is understood, therefore, that this invention is not limited to the particular embodiments disclosed, but is intended to cover all modifications which are within the spirit and scope of the invention as defined by the appended claims; the above description, and/or shown in the attached drawings. Consequently only such limitations as appear in the appended claims should be placed on the disclosure.

What is claimed is:

1. A method of preparing a mRNA transcript expression library from a tissue sample comprising,
a) mounting the tissue sample on a substrate comprising a plurality of capture oligonucleotides, wherein the capture oligonucleotides comprise a first clustering sequence, a spatial barcode sequence (SBC) and a first universal adapter sequence;
b) contacting the tissue sample with i) a plurality of 5′ gene specific probes comprising a sequence complementary to the first universal adapter sequence and a 5′ gene specific primer; and ii) a plurality of 3′ gene specific probes comprising a 3′ gene specific primer, a unique molecular index, and a second universal adapter sequence, under conditions such that one or more 5′ gene specific probe and one or more 3′ gene specific probe hybridizes to one or more mRNA transcript in the tissue sample;
c) contacting the tissue sample in (b) with ligation reagents such that a 5′ gene specific probe and a 3′ gene specific probe hybridized to the mRNA transcript in proximity to each other are ligated together to form one or more ligated gene specific probe pairs;
d) removing the mRNA transcript hybridized to the ligated gene specific probe pairs and leaving a ligated gene specific probe pair oligonucleotide sequence;
e) capturing the ligated gene specific probe pair oligonucleotide of (d) on the substrate by binding of the sequence complementary to the first universal adapter sequence in the 5′ gene specific probe to the first universal adapter sequence of the capture oligonucleotide.

2. A method of determining mRNA transcript expression in a tissue sample comprising,
a) mounting the tissue sample on a substrate comprising a plurality of capture oligonucleotides, wherein the capture oligonucleotides comprise a first clustering sequence, a spatial barcode sequence (SBC) and a first universal adapter sequence;
b) contacting the tissue sample with i) a plurality of 5′ gene specific probes comprising a sequence complementary to the first universal adapter sequence and a 5′ gene specific primer; and ii) a plurality of 3′ gene specific probes comprising a 3′ gene specific primer, a unique molecular index, and a second universal adapter sequence, under conditions such that one or more 5′ gene specific probe and one or more 3′ gene specific probe hybridizes to one or more mRNA transcript in the tissue sample;
c) contacting the tissue sample in (b) with ligation reagents such that a 5′ gene specific probe and a 3′ gene specific probe hybridized to the mRNA transcript in proximity to each other are ligated together to form one or more ligated gene specific probe pairs;
d) removing mRNA transcripts hybridized to ligated gene specific probe pairs and leaving ligated gene specific probe pair oligonucleotide sequences;
e) capturing the ligated gene specific probe pair oligonucleotide of (d) on the substrate by binding of the sequence complementary to the first universal adapter sequence in the 5′ gene specific probe to the first universal adapter sequence of the capture oligonucleotide.

3. The method of claim 1 or 2, wherein the 3′ gene specific probe comprises one or more ribobases.

4. A method of preparing a mRNA transcript expression library from a tissue sample comprising,
a) mounting the tissue sample on a substrate comprising a plurality of capture oligonucleotides, wherein the capture oligonucleotides comprise a first clustering sequence, a spatial barcode sequence (SBC) and a first universal adapter sequence;
b) contacting the tissue sample with i) a plurality of 5′ gene specific probes comprising a sequence complementary to the first universal adapter sequence and a 5′ gene specific primer; and ii) a plurality of 3′ gene specific probes comprising a 3′ gene specific primer, a unique molecular index, and a second universal adapter sequence, under conditions such that one or more 5′ gene specific probe and one or more 3′ gene specific probe hybridizes to one or more mRNA transcript in the tissue sample,
wherein hybridization of the 5′ gene specific probe and 3′ gene specific probe on the mRNA transcript results in a nucleotide gap between the hybridized molecules;
c) contacting the tissue sample in (b) with nucleotide bases and ligation reagents such that the nucleotide gap between the 5′ gene specific probe and 3′ gene specific probe hybridized to the mRNA transcript is filled with nucleotide bases complementary to the mRNA transcript, and a 5′ gene specific probe and a 3′ gene specific probe are ligated together to form one or more ligated gene specific probe pairs;
d) removing mRNA transcripts hybridized to ligated gene specific probe pairs and leaving ligated gene specific probe pair oligonucleotide sequences;
e) capturing the ligated gene specific probe pair oligonucleotide sequences of (d) on the substrate by binding of the sequence complementary to the first universal adapter sequence in the 5′ gene specific probe to the first universal adapter sequence of the capture oligonucleotide.

5. A method of isolating mRNA transcript expression in a tissue sample comprising,
a) mounting the tissue sample on a substrate comprising a plurality of capture oligonucleotides, wherein the capture oligonucleotides comprise a first clustering sequence, a spatial barcode sequence (SBC) and a first universal adapter sequence;
b) contacting the tissue sample with i) a plurality of 5′ gene specific probes comprising a sequence complementary to the first universal adapter sequence and a 5′ gene specific primer; and ii) a plurality of 3′ gene specific probes comprising a 3′ gene specific primer, a unique molecular index, and a second universal adapter sequence, under conditions such that one or more 5′ gene specific probe and one or more 3′ gene specific probe hybridizes to one or more mRNA transcript in the tissue sample,
wherein hybridization of the one or more 5′ gene specific probe and one or more 3″ gene specific probe on the mRNA transcript results in a nucleotide gap between the hybridized molecules;
c) contacting the tissue sample in (b) with nucleotide bases and ligation reagents such that the nucleotide gap between a 5′ gene specific probe and a 3′ gene specific probe hybridized to the mRNA transcript is filled with nucleotide bases complementary to the mRNA transcript, and the 5′ gene specific probe and 3′ gene specific probe are ligated together to form one or more ligated gene specific probe pairs;
d) removing mRNA transcripts hybridized to the ligated gene specific probe pairs and leaving ligated gene specific probe pair oligonucleotide sequences;
e) capturing the ligated gene specific probe pair oligonucleotide sequences of (d) on the substrate by binding of the sequence complementary to the first universal adapter sequence in the 5′ gene specific probe to the first universal adapter sequence of the capture oligonucleotide.

6. The method of claim 4 or 5 wherein the nucleotide gap is from 1 to 50 or more nucleotides.

7. The method of any one of claims 1 to 6 further comprising indexing and sequencing the ligated gene specific probe pairs comprising,
f) performing extension reactions and PCR on the oligonucleotide of (e) to yield a PCR template representative of one or more mRNA transcripts in the tissue sample;
g) eluting the PCR template;
h) carrying out an indexing PCR to generate a double stranded PCR product comprising the first strand PCR product and a second strand complementary to the first strand PCR product.

8. The method of claim 7 further comprising sequencing the PCR product of (h) and determining the location of the mRNA transcript in the tissue based on the spatial barcode of (a).

9. The method of claim 7 or 8, wherein the double stranded PCR product comprises a second clustering sequence on the second strand complementary to the first strand PCR product and, optionally, an index sequence.

10. The method of any one of claims 1-9, wherein the 5′ gene specific probe and/or the 3′ gene specific probe is between 10-50 nucleotides.

11. The method of any one of claims 1-10, wherein the first clustering sequence comprises a P7 sequence.

12. The method of any one of claims 1-11, wherein the first universal adapter sequence comprises GTGACTGGAGTTCAGACGTGTGCTCTTCCGATCT (SEQ ID NO: 19).

13. The method of any one of claims 1-12, wherein the second universal adapter sequence comprises AGATCGGAAGAGCGTCGTGTAGGGAAAGAGTG (SEQ ID NO: 20).

14. The method of any one of claims 1-13, wherein the 5′ gene specific probe and/or the 3′ gene specific probe have a melting temperature (Tm) of about 50-55° C.

15. The method of any one of claims 1-14, wherein the capture oligonucleotides have a melting temperature (Tm) of about 40-42° C.

16. The method of any one of claims 1-15, wherein step (b) is carried out at approximately 50-55° C.

17. The method of any one of claims 1-16, wherein step (e) is carried out at approximately 40-42° C.

18. The method of any one of claims 1-17, wherein contacting the tissue sample with the substrate correlates a position of a capture site on the substrate with a position in the tissue sample, wherein the substrate comprises a plurality of capture sites comprising a plurality of capture probes immobilized on a surface, wherein the capture probes comprise a spatial address region.

19. The method of any one of claims 1-18, wherein the sample is from a mammal.

20. The method of any one of claims 1-19, wherein the sample is from a human.

21. The method of any one of claims 1-20, wherein the tissue sample is a tumor biopsy.

22. The method of any one of claims 1-21, wherein the tissue sample is formalin-fixed paraffin embedded (FFPE) tissue or fresh frozen (FF) tissue.

23. A method of identifying a genetic variation in a subject having or at risk of having a disease comprising,
i) generating a sample mRNA library from a tissue sample from the subject according to the methods of any one of claims 1-21,
i) comparing the genetic information from the sample mRNA library to a control mRNA library, and
iii) identifying a genetic variation in the sample mRNA library associated with the disease.

24. The method of claim 23, wherein the disease is a genetic defect, cancer, an autoimmune disease, or a metabolic disorder.

25. The method of claim 23 or 24, wherein the disease is cancer.

26. A method for preparing a spatially barcoded RNA library from a tissue sample comprising,
(a) contacting the tissue sample with a plurality of RNA capture probes that hybridize with RNA in the tissue sample, wherein each of the RNA capture probes comprises an RNA capture oligonucleotide sequence complementary to an RNA in the sample and a first substrate capture oligonucleotide complementary to a first domain of a plurality of splint oligonucleotides;
(b) hybridizing the RNA capture oligonucleotide of the RNA capture probes with RNA in the tissue sample to form RNA-RNA capture probe hybrids;
(c) carrying out extension of the RNA capture oligonucleotide of the RNA-RNA capture probe hybrids using reverse transcriptase to form a plurality of first strand cDNA molecules, wherein each of the first strand cDNA molecules comprises the RNA capture oligonucleotide and the first substrate capture oligonucleotide;
(d) capturing the first strand cDNA molecules on a substrate, wherein the substrate comprises a plurality of substrate capture probes each comprising a spatial barcode and a second substrate capture oligonucleotide complementary to a second domain of the splint oligonucleotides, and wherein the capturing comprises hybridizing the splint oligonucleotides with the first substrate capture oligonucleotide of the first strand cDNA molecules and the second substrate capture oligonucleotide of the substrate capture probes; and
(e) ligating the captured first strand cDNA molecules to the substrate capture probes, thereby forming spatially barcoded first strand cDNA molecules.

27. The method of claim 26, wherein the substrate capture probe further comprises a substrate anchor moiety.

28. The method of claim 26 or 27, wherein the surface oligonucleotide further comprises a P7 adapter and the RNA capture probe primer for reading the spatial barcode sequence.

29. A method for preparing a spatially barcoded RNA library from a tissue sample comprising,
(a) contacting the tissue sample with a plurality of RNA capture probes that hybridize with RNA in the tissue sample, wherein the RNA capture probes comprise an RNA capture oligonucleotide complementary to an RNA in the sample and a handle sequence;
(b) hybridizing the RNA capture oligonucleotide of the RNA capture probes with RNA in the tissue sample to form RNA-RNA capture probe hybrids;
(c) carrying out extension of the RNA capture oligonucleotide of the RNA-RNA capture probe hybrids using reverse transcriptase to form a plurality of first strand cDNA molecules, wherein each of the first strand cDNA molecules comprises the RNA capture oligonucleotide and the handle sequence;
(d) adding a 3′ end oligonucleotide to the 3′ end of each first strand cDNA molecule, wherein the 3′ end oligonucleotide comprises a substrate capture oligonucleotide complementary to a first domain of a plurality of substrate capture probes on a substrate, wherein each of the plurality of substrate capture probes comprises, in the 5′ to 3′ orientation, a substrate anchor sequence, a spatial barcode, and the first domain;
(e) hybridizing the substrate capture oligonucleotide of the first strand cDNA molecules with the first domain of the substrate capture probes; and
(f) carrying out extension of the first domain of the hybridized substrate capture probes to form a plurality of spatially barcoded first strand cDNA molecules.

30. The method of claim 29, wherein the handle sequence is a PCR handle sequence, a molecular identifier, a UMI, or any combination thereof.

31. The method of claim 29 or 30, wherein the handle sequence is a P5 adapter sequence.

32. The method of any one of claims 29-31, wherein the 3′ end oligonucleotide is added by tagmentation.

33. The method of any one of claims 29-32, wherein the 3′ end oligonucleotide is added by click chemistry, or oNTP-directed adapterization.

34. The method of claim 33, wherein the 3′OH is added by terminating the extension reaction with a click labeled nucleotide.

35. The method of claim 34, wherein the click labeled nucleotide is an azide or alkyne labeled oligonucleotide.

36. The method of claim 34 or 35, wherein the extension reaction adds a poly A sequence to the 3′ extended sequence.

37. The method of any one of claims 29-36, wherein the first strand cDNA is captured with a polyT sequence on the surface capture oligonucleotide.

38. A method for preparing a spatially barcoded RNA library from a tissue sample comprising,
(a) contacting the tissue sample with a plurality of RNA capture probes that hybridize with RNA in the tissue sample, wherein the RNA capture probes comprise an RNA capture oligonucleotide complementary to an RNA in the sample and a handle sequence;
(b) hybridizing the RNA capture oligonucleotide of the RNA capture probes with RNA in the tissue sample to form RNA-RNA capture probe hybrids;
(c) carrying out extension of the RNA capture oligonucleotide of the RNA-RNA capture probe hybrids using reverse transcriptase to form a plurality of first strand cDNA molecules, wherein each of the first strand cDNA molecules comprises the RNA capture oligonucleotide and the handle sequence;
(d) adding a 3′ end oligonucleotide to the 3′ end of each first strand cDNA molecule, via template switching, comprising contacting the first strand cDNA molecule with a reverse transcriptase (RT) and a template switch oligonucleotide (TSO), wherein the RT incorporates untemplated cytosine nucleotides at the 3′ end of the first cDNA and the TSO comprises a sequence capable of hybridizing to the untemplated cytosine nucleotides, wherein the 3′ end oligonucleotide is appended to the 3′ end of the first cDNA and the RT extends to generate a TSO complement;
wherein the 3′ end oligonucleotide comprises a substrate capture oligonucleotide complementary to a first domain of a plurality of substrate capture probes on a substrate, wherein each of the plurality of substrate capture probes comprises, in the 5′ to 3′ orientation, a substrate anchor sequence, a spatial barcode, and the first domain;
(e) hybridizing the substrate capture oligonucleotide of the first strand cDNA molecules with the first domain of the substrate capture probes; and
(f) carrying out extension of the first domain of the hybridized substrate capture probes to form a plurality of spatially barcoded first strand cDNA molecules.

39. The method of claim 38, wherein the first domain is a poly T sequence.

40. A method for preparing a spatially barcoded RNA library from a tissue sample comprising,
(a) contacting the tissue sample with a plurality of RNA capture probes that hybridize with RNA in the tissue sample, wherein the RNA capture probes comprise an RNA capture oligonucleotide complementary to an RNA in the sample and a handle sequence;
(b) hybridizing the RNA capture oligonucleotide of the RNA capture probes with RNA in the tissue sample to form RNA-RNA capture probe hybrids;
(c) carrying out extension of the RNA capture oligonucleotide of the RNA-RNA capture probe hybrids using reverse transcriptase to form a plurality of first strand cDNA molecules, wherein each of the first strand cDNA molecules comprises the RNA capture oligonucleotide and the handle sequence;
(d) adding a 3′ end oligonucleotide to the 3′ end of each first strand cDNA molecule, via template switching, comprising contacting the first strand cDNA molecule with a reverse transcriptase (RT) and a template switch oligonucleotide (TSO), wherein the RT incorporates untemplated cytosine nucleotides at the 3′ end of the first cDNA and the TSO comprises a sequence capable of hybridizing to the untemplated cytosine nucleotides, wherein the 3′ end oligonucleotide is appended to the 3′ end of the first cDNA and the RT extends to generate a TSO complement;
wherein the 3′ end oligonucleotide comprises a substrate capture oligonucleotide complementary to a first domain of a plurality of substrate capture probes on a substrate, wherein each of the plurality of substrate capture probes comprises, in the 5′ to 3′ orientation, a substrate anchor sequence, a second handle, a spatial barcode, and the first domain;
(e) releasing the substrate capture probes from the substrate;
(f) hybridizing the substrate capture oligonucleotide of the first strand cDNA molecules with the first domain of the substrate capture probes; and
(g) contacting the first strand with a second strand synthesis mix comprising a TSO primer and extending the TSO primer using the first strand as a template to generate a second strand complementary to the first strand, the second strand comprising the TSO, a second cDNA complementary to the first cDNA, and second strand barcode information comprising a spatial barcode sequence complement (SBC′) that is complementary to the spatial barcode sequence (SBC).

41. The method of claim 40, wherein the first domain is a poly G sequence that hybridizes with the poly C sequence on the TSO.

42. The method of claim 40 or 41, wherein the handle is a P5 sequence and the second handle is a P7 sequence.

43. A method for preparing a spatially barcoded RNA library from a tissue sample comprising,
(a) contacting the tissue sample with a plurality of RNA capture probes that bind RNA in the tissue sample, wherein each of the RNA capture probes comprise a RNA capture oligonucleotide complementary to an RNA in the sample and a substrate capture oligonucleotide complementary to a first domain of a plurality of substrate capture probes on a substrate, wherein the RNA capture oligonucleotide complementary to the RNA is blocked on the 3′ end; wherein each of the substrate capture probes comprises, in the 5′ to 3′ orientation, the first domain and a first substrate anchor sequence and is in proximity to one or more barcoded substrate probes on the substrate, and wherein each of the barcoded substrate probes comprises, in the 5′ to 3′ orientation, a second substrate anchor sequence, a spatial barcode, and a random priming sequence;
(b) hybridizing the RNA capture oligonucleotide of the RNA capture probes with RNA in the tissue sample to form RNA-RNA capture probe hybrids having a 5′ single-stranded RNA region;
(c) hybridizing the substrate capture oligonucleotide of the RNA-RNA capture probe hybrids with the first domain of the substrate capture probes (d) hybridizing the 5′ single-stranded RNA region of the RNA-RNA capture probe hybrids with the random priming sequence of the barcoded substrate probes; and
(e) carrying out extension of the random priming sequences hybridized to the 5′ single-stranded RNA regions using reverse transcriptase to form a plurality of spatially barcoded first strand cDNA molecules.

44. The method of claim 43, wherein the nucleotide sequence complementary to RNA in the sample is a polyT oligonucleotide, a randomer, a semi-randomer, or a target specific sequence.

45. The method of claim 43 or 44, wherein the nucleotide sequence complementary to an RNA in the sample is a polyT oligonucleotide.

46. The method of claim 43, wherein the RNA is removed from the sample.

47. The method of claim 46, wherein the RNA is removed from the sample after extension to form first strand cDNA.

48. The method of claim 47, wherein the RNA is removed by enzymatic or thermal methods.

49. A method for preparing a spatially barcoded RNA library from a tissue sample comprising,
(a) contacting the tissue sample with a plurality of RNA capture probes that hybridize with RNA in the tissue sample, wherein each of the RNA capture probes comprises an RNA capture oligonucleotide complementary to an RNA in the sample and a substrate capture oligonucleotide complementary to a first domain of a plurality of substrate capture probes on a substrate, wherein each of the substrate capture probes comprises, in the 5′ to 3′ orientation, a substrate anchor sequence, the first domain, a linker, a spatial barcode, and a random priming sequence;
(b) hybridizing the RNA capture probes with the RNA in the tissue sample to form RNA-RNA capture probe hybrids having a 5′ single-stranded RNA region;
(c) hybridizing the substrate capture oligonucleotide of the RNA-RNA capture probe hybrids with the first domain of the substrate capture probes;
(d) hybridizing the 5′ single-stranded RNA regions of the RNA-RNA capture probe hybrids with the random priming sequence of the substrate capture probes; and
(e) carrying out extension of the random priming sequences hybridized to the 5′ single-stranded RNA regions using reverse transcriptase to form a plurality of spatially barcoded first strand cDNA molecules.

50. The method of claim 49, wherein the linker is a linker that cannot be read through by a polymerase.

51. A method for preparing a spatially barcoded RNA library from a tissue sample comprising,
(a) contacting the tissue sample with a plurality of RNA capture probes that bind RNA in the tissue sample, wherein each of the RNA capture probes comprise an RNA capture oligonucleotide complementary to an RNA in the sample and a substrate capture oligonucleotide complementary to a first domain of a plurality of substrate capture probes on a substrate; wherein each of the substrate capture probes comprises, in the 5′ to 3′ orientation, the first domain and a first substrate anchor sequence and is in proximity to at least one of a plurality of barcoded substrate probes on the substrate, and wherein each barcoded substrate probe comprises, in the 5′ to 3′ orientation, a spatial barcode and a second substrate anchor sequence;
(b) hybridizing the RNA capture oligonucleotide of the RNA capture probes with RNA in the tissue sample to form RNA-RNA-capture probe hybrids;
(c) capturing the RNA-RNA capture probe hybrids on the substrate by hybridizing substrate capture oligonucleotide of the RNA-RNA capture probe hybrids with the first domain of the substrate capture probes;
(d) carrying out extension of the RNA capture oligonucleotide of the captured RNA-RNA capture probe hybrids using reverse transcriptase to form a plurality of first strand cDNA molecules; and
(e) ligating each of the first strand cDNA molecules to the proximal barcoded substrate probe, thereby forming spatially barcoded first strand cDNA molecules.

52. A method for preparing a spatially barcoded RNA library from a tissue sample comprising,
(a) contacting the tissue sample with a plurality of RNA capture probes that bind RNA in the tissue sample, wherein each of the RNA capture probes comprise an RNA capture oligonucleotide complementary to an RNA in the sample and a substrate capture oligonucleotide complementary to a first domain of a plurality of substrate capture probes on a substrate, wherein the RNA capture oligonucleotide complementary to the RNA is blocked on the 3′ end; wherein each of the substrate capture probes comprises, in the 5′ to 3′ orientation, the first domain and a first substrate anchor sequence and is in proximity to at least one of a plurality of barcoded substrate probes on the substrate, and wherein each barcoded substrate probe comprises, in the 5′ to 3′ orientation, a polyT sequence, a spatial barcode and a second substrate anchor sequence;
(b) hybridizing the RNA capture oligonucleotide of the RNA capture probes with RNA in the tissue sample to form RNA-RNA-capture probe hybrids;
(c) capturing the RNA-RNA capture probe hybrids on the substrate by hybridizing substrate capture oligonucleotide of the RNA-RNA capture probe hybrids with the first domain of the substrate capture probe;
(d) polyadenylating the RNA in the sample at the 3′ end; and
(e) carrying out extension of the RNA capture oligonucleotide of the captured RNA-RNA capture probe hybrids using reverse transcriptase to form a plurality of first strand cDNA molecules.

53. The method of claim 52, wherein the polyadenylation is carried out using polyA polymerase.

54. A method for preparing a spatially barcoded RNA library from a tissue sample comprising,
(a) contacting the tissue sample with a plurality of RNA capture probes that hybridize with RNA in the tissue sample, wherein each of the RNA capture probes has a hairpin structure and comprises an DNA capture oligonucleotide complementary to RNA in the sample and a substrate capture oligonucleotide complementary to a first domain of a plurality of substrate capture probes on a substrate, wherein the DNA capture oligonucleotide of the RNA capture probes comprises a single stranded region, and wherein each of the substrate capture probes comprises, in the 5′ to 3′ orientation, a substrate anchor sequence, a spatial barcode, the first domain, and a second domain, wherein the second domain comprises at least one RNA nucleotide or nucleoside;
(b) hybridizing the RNA capture probes with the RNA in the tissue sample to form RNA-RNA capture probe hybrids, wherein each of the RNA-RNA capture probe hybrids comprises a 5′ single-stranded RNA end region;
(c) capturing the substrate capture oligonucleotide of the RNA-RNA capture probe hybrids on the substrate by hybridizing the substrate capture oligonucleotide of the RNA-RNA capture probe hybrids with the first domain of the substrate capture probes;
(d) phosphorylating the 5′ single-stranded RNA end region of the captured RNA-RNA capture probe hybrids and contacting the captured RNA-RNA capture probe hybrids with a 5′ to 3′ riboexonuclease to digest the phosphorylated 5′ single-stranded RNA end region; and
(e) ligating the digested 5′ RNA end region of the captured RNA-RNA capture probe hybrids to the second domain of the substrate capture probes to form a plurality of DNA-RNA chimeras on the substrate.

55. The method of claim 54, wherein the ligating is carried out with T4 ligase.

56. The method of claim 54, wherein the RNA of the captured RNA-RNA capture probe hybrids is 5′ phosphorylated prior to ligation.

57. The method of claim 56 further comprising generating first strand cDNA from the plurality of DNA-RNA chimeras on the substrate.

58. The method of claim 57, wherein the first strand cDNAs can be hybridized from the surface and processed for sequencing.

59. The method of any one of claims 54-58, wherein the reverse transcription is carried out using a DNA random primer, optionally which comprises P5 adaptor.

60. The method of any one of claims 26-59, wherein the cDNA extension templates can be dehybridized from the RNA in the tissue by chemical, enzymatic, or thermal dehybridization.

61. The method of any one of claims 29-59, wherein the cDNA extension templates can be dehybridized from the RNA on a substrate by chemical, enzymatic, or thermal dehybridization.

62. The method of claim 60 or 61 wherein the dehybridization step occurs before or after the capturing step.

63. The method of any one of claims 26-62, wherein the tissue sample is formalin-fixed paraffin embedded (FFPE) tissue or fresh frozen (FF) tissue.

64. The method of claim 63, further comprising decrosslinking the FFPE sample, optionally wherein the decrosslinking is carried out using TE buffer, pH 9.

65. The method of any one of claims 26-64, wherein the RNA capture probe is selected from the group consisting of a poly-T sequence, a poly-U sequence, a randomer, a semi-random sequence, or a target-specific probe.

66. The method of claim 65, wherein the RNA capture probe is a poly-T sequence.

67. The method of claim 65 or 66, wherein the RNA capture probe comprises at least 10 deoxythymidine residues.

68. The method of claim 67, wherein the target-specific probes comprise a plurality of different target-specific RNA capture probe sequences.

69. The method of claim 68, wherein the target-specific probes comprise at least 10 nucleotides complementary to a nucleotide sequence of a target RNA.

70. The method of claim 68 or 69, wherein the RNA capture probe or surface capture probe is between 8 to 80 nucleotides.

71. The method of any one of claims 26-70, wherein the targeted probe is between 8-80 nucleotides or between 10-50 nucleotides.

72. The method of any one of claims 26-71, wherein the tissue sample is permeabilized prior to contacting the tissue sample with a plurality of RNA capture probes.

73. The method of any one of claims 26-72, wherein the tissue sample is treated with one or more blocking reagents prior to contacting the tissue sample with a plurality of RNA capture probes).

74. The method of any one of claims 26-73, wherein the tissue sample is permeabilized and treated with one or more blocking reagents prior to contacting the tissue sample with a plurality of RNA capture probes).

75. The method of any one of claims 26-74, wherein the substrate is a bead, a bead array, a spotted array, a substrate comprising a plurality of wells, a flow cell, clustered particles arranged on a surface of a chip, a film, or a plate.

76. The method of claim 75, wherein the substrate comprises a plurality of nanowells or microwells.

77. The method of any one of claims 26-76, wherein the spatially barcoded first strand cDNA molecules are recovered by contacting the spatially barcoded first strand cDNAs on the substrate with a DNA polymerase and one or more primers to generate spatially barcoded second strand cDNAs complementary to the spatially barcoded first strand cDNAs and removing the spatially barcoded second strand cDNAs from the substrate.

78. The method of claim 77, wherein the one or more primers each comprise a random priming sequence.

79. The method of claim 78, wherein the random priming sequences comprises nine random nucleotides.

80. The method of claim 78 or 79, wherein the spatially barcoded second strand cDNAs each comprise a unique molecular identifier (UMI), wherein the UMI comprises an intrinsic sequence and an extrinsic sequence, wherein the extrinsic sequence is a sequence complementary to the random priming sequence used to generate the second strand cDNA, and wherein the intrinsic sequence is a sequence complementary to the first strand cDNA template sequence used to generate the second strand cDNA.

81. The method of claim 77, wherein the one or more primers each comprise a molecular identifier barcode.

82. The method of claim 77, wherein the one or more primers each comprise a UMI barcode.

83. The method of any one of claims 77-82, wherein the spatially barcoded second strand cDNAs are removed from the substrate by chemical or physical dehybridization.

84. The method of any one of claims 77-83, wherein the anchor sequence comprises a cleavage site, and hybrids of the spatially barcoded first and second strand cDNAs are removed from the substrate by enzymatic cleavage at the cleavage site.

85. The method of claim 84, wherein the cleavage site is a binding site for a restriction endonuclease.

86. The method of claim 84, wherein the anchor sequence comprises a cleavage site, and wherein the spatially barcoded first strand cDNA molecules are recovered by enzymatic cleavage at the cleavage site.

87. The method of claim 86, wherein the cleavage site is a binding site for a restriction endonuclease.

88. The method of any one of claims 77-87, further comprising sequencing at least a portion of the cDNA libraries to determine the spatial barcode sequence for each molecule.

89. The method of claim 88, further comprising determining the spatial location of one or more cDNA molecules by correlating the spatial barcode sequences of the one or more cDNA molecules with the spatial locations of the surface oligonucleotide molecules on the substrate containing corresponding spatial barcode sequences.

90. The method of any one of claims 26 to 89 further comprising indexing and sequencing spatially barcoded first strand cDNAs, comprising,
performing extension reactions and PCR on the spatially barcoded first strand cDNAs to yield a PCR template comprising a first strand PCR product representative of one or more RNA transcripts in the tissue sample;
eluting the PCR template;
carrying out an indexing PCR to generate a double stranded PCR product comprising the first strand PCR product and a second strand complementary to the first strand PCR product.

91. The method of claim 90 further comprising sequencing the PCR product and determining the location of the RNA transcript in the tissue based on the spatial barcode of first strand cDNA.

92. The method of claim 90 or 91, wherein the double stranded PCR product comprises a second clustering sequence on the second strand complementary to the first strand PCR product and, optionally, an index sequence.

93. The method of claim 91 or 92, wherein the PCR products are further processed by tagmentation to generate a spatial transcriptomics library.

94. The method of claim 93, wherein the tagmentation comprises on substrate tagmentation.

95. The method of any one of claims 26 to 94, wherein the methods determine RNA expression in a single cell with the tissue sample.

96. The method of claim 95, wherein the methods determine RNA expression in one or more subcellular components in the single cell.

97. The method of claim 96, wherein the subcellular component is a cell nucleus, cytoplasm, or mitochondria.

98. The method of any one of the preceding claims, wherein the substrate or surface of the substrate comprises a material selected from glass, silicon, poly-L-lysine coated materials, nitrocellulose, polystyrene, cyclic olefin copolymers (COCs), cyclic olefin polymers (COPs), polyacrylamide, polypropylene, polyethylene, or polycarbonate

99. The method of any one of claims 26 to 98, wherein the RNA library is an mRNA library.
